SEQUENCE LISTING (100 sequences)

SEQ ID NO: 1 - DNA, 70 bp, synthetic construct
  source 1..70: mol_type = other DNA
ggacactctttccctacacgacgctcttccgatctnantnancntntncntnanatgggaaagagtgtcc

SEQ ID NO: 2 - DNA, 14 bp, synthetic construct
  source 1..14: mol_type = other DNA
ggacactctttccc

SEQ ID NO: 3 - DNA, 14 bp, synthetic construct
  source 1..14: mol_type = other DNA
gggaaagagtgtcc

SEQ ID NO: 4 - DNA, 35 bp, synthetic construct
  source 1..35: mol_type = other DNA
ggacactctttccctacacgacgctcttccgatct

SEQ ID NO: 5 - DNA, 34 bp, synthetic construct
  source 1..34: mol_type = other DNA
gtgactggagttcagacgtgtgctcttccgatct

SEQ ID NO: 6 - DNA, 20 bp, synthetic construct
  source 1..20: mol_type = other DNA
aatgatacggcgaccaccga

SEQ ID NO: 7 - DNA, 29 bp, synthetic construct
  source 1..29: mol_type = other DNA
aatgatacggcgaccaccgagatctacac

SEQ ID NO: 8 - DNA, 24 bp, synthetic construct
  source 1..24: mol_type = other DNA
caagcagaagacggcatacgagat

SEQ ID NO: 9 - DNA, 63 bp, synthetic construct
  source 1..63: mol_type = other DNA
ggacactctttccctacacgacgctcttccgatctnnnnnnnnnnnnatgggaaagagtgtcc

SEQ ID NO: 10 - DNA, 34 bp, synthetic construct
  source 1..34: mol_type = other DNA
tgctcattttgaagtaaatccagattgttctgtt

SEQ ID NO: 11 - DNA, 31 bp, synthetic construct
  source 1..31: mol_type = other DNA
actgcaagtcgagaggtattcagaatacatt

SEQ ID NO: 12 - DNA, 17 bp, synthetic construct
  source 1..17: mol_type = other DNA
tgggacccactccatcg

SEQ ID NO: 13 - DNA, 28 bp, synthetic construct
  source 1..28: mol_type = other DNA
atatttcttcatgaagacctcacagtaa

SEQ ID NO: 14 - DNA, 16 bp, synthetic construct
  source 1..16: mol_type = other DNA
caactgcgccgacccc

SEQ ID NO: 15 - DNA, 18 bp, synthetic construct
  source 1..18: mol_type = other DNA
cagcgtgtccaggaagcc

SEQ ID NO: 16 - DNA, 20 bp, synthetic construct
  source 1..20: mol_type = other DNA
ttgcacactgtcagcaaaac

SEQ ID NO: 17 - DNA, 21 bp, synthetic construct
  source 1..21: mol_type = other DNA
accaataactccaaggctggt

SEQ ID NO: 18 - DNA, 26 bp, synthetic construct
  source 1..26: mol_type = other DNA
gtcactggcagcaacagtcttacctg

SEQ ID NO: 19 - DNA, 28 bp, synthetic construct
  source 1..28: mol_type = other DNA
ttcctcaggattgcctttaccactcaga

SEQ ID NO: 20 - DNA, 24 bp, synthetic construct
  source 1..24: mol_type = other DNA
tggcctgcaggattatgtcttatt

SEQ ID NO: 21 - DNA, 22 bp, synthetic construct
  source 1..22: mol_type = other DNA
ccaggaaacgcacaacactcag

SEQ ID NO: 22 - DNA, 23 bp, synthetic construct
  source 1..23: mol_type = other DNA
ctgaatacaggttatcagtgcct

SEQ ID NO: 23 - DNA, 27 bp, synthetic construct
  source 1..27: mol_type = other DNA
tcaaagatcctgtgcagaaaaatgaat

SEQ ID NO: 24 - DNA, 25 bp, synthetic construct
  source 1..25: mol_type = other DNA
tgcagagttcagttaccttaggaga

SEQ ID NO: 25 - DNA, 22 bp, synthetic construct
  source 1..22: mol_type = other DNA
tgcatacatcagacagcacaga

SEQ ID NO: 26 - DNA, 23 bp, synthetic construct
  source 1..23: mol_type = other DNA
ccatgacaagattttcccttacc

SEQ ID NO: 27 - DNA, 25 bp, synthetic construct
  source 1..25: mol_type = other DNA
caccttatatgggcatacttccact

SEQ ID NO: 28 - DNA, 18 bp, synthetic construct
  source 1..18: mol_type = other DNA
ccaccttgggctacctgc

SEQ ID NO: 29 - DNA, 20 bp, synthetic construct
  source 1..20: mol_type = other DNA
ggtactcgatgatgccggtg

SEQ ID NO: 30 - DNA, 23 bp, synthetic construct
  source 1..23: mol_type = other DNA
gtcctttcaggatggtggatgtg

SEQ ID NO: 31 - DNA, 23 bp, synthetic construct
  source 1..23: mol_type = other DNA
ggatgtcacgttctcaaagcagt

SEQ ID NO: 32 - DNA, 21 bp, synthetic construct
  source 1..21: mol_type = other DNA
attcgatgatccctgtggtgg

SEQ ID NO: 33 - DNA, 20 bp, synthetic construct
  source 1..20: mol_type = other DNA
gtagctgaccctgcctacct

SEQ ID NO: 34 - DNA, 25 bp, synthetic construct
  source 1..25: mol_type = other DNA
gcagtgtatccattttcttctctct

SEQ ID NO: 35 - DNA, 25 bp, synthetic construct
  source 1..25: mol_type = other DNA
gtattgttaaccttgcagaatggtc

SEQ ID NO: 36 - DNA, 21 bp, synthetic construct
  source 1..21: mol_type = other DNA
gcgatgacggaatataagctg

SEQ ID NO: 37 - DNA, 20 bp, synthetic construct
  source 1..20: mol_type = other DNA
ctggatcagctggatggtca

SEQ ID NO: 38 - DNA, 27 bp, synthetic construct
  source 1..27: mol_type = other DNA
ccaacatgacttacttgatccccataa

SEQ ID NO: 39 - DNA, 25 bp, synthetic construct
  source 1..25: mol_type = other DNA
cttgtgagtggatgggtaaaaccta

SEQ ID NO: 40 - DNA, 19 bp, synthetic construct
  source 1..19: mol_type = other DNA
agtggatcccctctccacc

SEQ ID NO: 41 - DNA, 20 bp, synthetic construct
  source 1..20: mol_type = other DNA
tagtccctggctggaccaag

SEQ ID NO: 42 - DNA, 18 bp, synthetic construct
  source 1..18: mol_type = other DNA
agggagctgcaggttctg

SEQ ID NO: 43 - DNA, 19 bp, synthetic construct
  source 1..19: mol_type = other DNA
gccatcgctgtagaacgca

SEQ ID NO: 44 - DNA, 20 bp, synthetic construct
  source 1..20: mol_type = other DNA
gcagatgtcaagccctccaa

SEQ ID NO: 45 - DNA, 20 bp, synthetic construct
  source 1..20: mol_type = other DNA
gctgaccccaaagtcacaga

SEQ ID NO: 46 - DNA, 20 bp, synthetic construct
  source 1..20: mol_type = other DNA
cgccttgaggcctttcttac

SEQ ID NO: 47 - DNA, 21 bp, synthetic construct
  source 1..21: mol_type = other DNA
gtcgtcatccttcagttctcc

SEQ ID NO: 48 - DNA, 25 bp, synthetic construct
  source 1..25: mol_type = other DNA
aacctctctttcttccacctttctc

SEQ ID NO: 49 - DNA, 24 bp, synthetic construct
  source 1..24: mol_type = other DNA
cttatgatctggttccggattgcg

SEQ ID NO: 50 - DNA, 27 bp, synthetic construct
  source 1..27: mol_type = other DNA
tcatcagttcctcctcagaattccccc

SEQ ID NO: 51 - DNA, 29 bp, synthetic construct
  source 1..29: mol_type = other DNA
aagcatacctggtataaacagtggcacac

SEQ ID NO: 52 - DNA, 24 bp, synthetic construct
  source 1..24: mol_type = other DNA
gctggattgtcagtgcgcttttcc

SEQ ID NO: 53 - DNA, 27 bp, synthetic construct
  source 1..27: mol_type = other DNA
gactgagtacaaactggtggtggttgg

SEQ ID NO: 54 - DNA, 20 bp, synthetic construct
  source 1..20: mol_type = other DNA
cgcatttaccccaagggagg

SEQ ID NO: 55 - DNA, 21 bp, synthetic construct
  source 1..21: mol_type = other DNA
cagccagcgttccagaaaatc

SEQ ID NO: 56 - DNA, 21 bp, synthetic construct
  source 1..21: mol_type = other DNA
agccttcgtgcactagttgat

SEQ ID NO: 57 - DNA, 25 bp, synthetic construct
  source 1..25: mol_type = other DNA
gtgcctttctaaattgcagtttgtt

SEQ ID NO: 58 - DNA, 31 bp, synthetic construct
  source 1..31: mol_type = other DNA
acttgcctactgatcagttacacaaccaatg

SEQ ID NO: 59 - DNA, 36 bp, synthetic construct
  source 1..36: mol_type = other DNA
ggttaattcccaatttaagatacttacacagtaggg

SEQ ID NO: 60 - DNA, 21 bp, synthetic construct
  source 1..21: mol_type = other DNA
ggctcccataactgcctgaat

SEQ ID NO: 61 - DNA, 23 bp, synthetic construct
  source 1..23: mol_type = other DNA
ggatttggttgtgaagattcgct

SEQ ID NO: 62 - DNA, 24 bp, synthetic construct
  source 1..24: mol_type = other DNA
ccatgtaagtcctagcattcattt

SEQ ID NO: 63 - DNA, 23 bp, synthetic construct
  source 1..23: mol_type = other DNA
tgtactctttttcctgttgctgt

SEQ ID NO: 64 - DNA, 22 bp, synthetic construct
  source 1..22: mol_type = other DNA
gaggctacaacagcaaaagctc

SEQ ID NO: 65 - DNA, 25 bp, synthetic construct
  source 1..25: mol_type = other DNA
ctatgatctctaccatgagacctga

SEQ ID NO: 66 - DNA, 21 bp, synthetic construct
  source 1..21: mol_type = other DNA
agatggcacagcccataagaa

SEQ ID NO: 67 - DNA, 19 bp, synthetic construct
  source 1..19: mol_type = other DNA
gaagtcctggcaagcgaga

SEQ ID NO: 68 - DNA, 20 bp, synthetic construct
  source 1..20: mol_type = other DNA
gcaatggccaaagcactgat

SEQ ID NO: 69 - DNA, 23 bp, synthetic construct
  source 1..23: mol_type = other DNA
cctgtgtttggttttgtaggtct

SEQ ID NO: 70 - DNA, 25 bp, synthetic construct
  source 1..25: mol_type = other DNA
accatcaatcagttgttcttcaagt

SEQ ID NO: 71 - DNA, 23 bp, synthetic construct
  source 1..23: mol_type = other DNA
aaatggtgatggagacaattgag

SEQ ID NO: 72 - DNA, 21 bp, synthetic construct
  source 1..21: mol_type = other DNA
gtacgtcactgctctgcgccg

SEQ ID NO: 73 - DNA, 28 bp, synthetic construct
  source 1..28: mol_type = other DNA
ggtaggaatggagggaagagaacctgaa

SEQ ID NO: 74 - DNA, 18 bp, synthetic construct
  source 1..18: mol_type = other DNA
gtggcaagtggctcctga

SEQ ID NO: 75 - DNA, 16 bp, synthetic construct
  source 1..16: mol_type = other DNA
catgggcggcatgaac

SEQ ID NO: 76 - DNA, 19 bp, synthetic construct
  source 1..19: mol_type = other DNA
ggcccctgtcatcttctgt

SEQ ID NO: 77 - DNA, 20 bp, synthetic construct
  source 1..20: mol_type = other DNA
cagaatgcaagaagcccaga

SEQ ID NO: 78 - DNA, 20 bp, synthetic construct
  source 1..20: mol_type = other DNA
ttgcgtgtggagtatttgga

SEQ ID NO: 79 - DNA, 20 bp, synthetic construct
  source 1..20: mol_type = other DNA
agacctcaggcggctcatag

SEQ ID NO: 80 - DNA, 27 bp, synthetic construct
  source 1..27: mol_type = other DNA
cttccagtgtgatgatggtgaggatgg

SEQ ID NO: 81 - DNA, 26 bp, synthetic construct
  source 1..26: mol_type = other DNA
acatgtgtaacagttcctgcatgggc

SEQ ID NO: 82 - DNA, 20 bp, synthetic construct
  source 1..20: mol_type = other DNA
tcctctgttgctgcagatcc

SEQ ID NO: 83 - DNA, 20 bp, synthetic construct
  source 1..20: mol_type = other DNA
tgagttccaaggcctcattc

SEQ ID NO: 84 - DNA, 22 bp, synthetic construct
  source 1..22: mol_type = other DNA
cctggtgtgggtcttcaggtgg

SEQ ID NO: 85 - DNA, 32 bp, synthetic construct
  source 1..32: mol_type = other DNA
gtgtgaaaccattccagtgtaaaacttgtcag

SEQ ID NO: 86 - DNA, 18 bp, synthetic construct
  source 1..18: mol_type = other DNA
gtggcaagtggctcctga

SEQ ID NO: 87 - DNA, 16 bp, synthetic construct
  source 1..16: mol_type = other DNA
catgggcggcatgaac

SEQ ID NO: 88 - DNA, 14 bp, synthetic construct
  source 1..14: mol_type = other DNA
sswswwwswwswss

SEQ ID NO: 89 - DNA, 21 bp, synthetic construct
  source 1..21: mol_type = other DNA
swsswwwsswswwwswwswss

SEQ ID NO: 90 - DNA, 23 bp, synthetic construct
  source 1..23: mol_type = other DNA
nantnancntntncntnantnan

SEQ ID NO: 91 - DNA, 73 bp, synthetic construct
  source 1..73: mol_type = other DNA
ggacactctttccctacacgacgctcttccgatctatgggaaagagtgtcccttacctgaggagacggtgacc

SEQ ID NO: 92 - DNA, 59 bp, synthetic construct
  source 1..59: mol_type = other DNA
gtgactggagttcagacgtgtgctcttccgatcttggagctgagcagcctgagatctga

SEQ ID NO: 93 - DNA, 59 bp, synthetic construct
  source 1..59: mol_type = other DNA
gtgactggagttcagacgtgtgctcttccgatctcaatgaccaacatggaccctgtgga

SEQ ID NO: 94 - DNA, 59 bp, synthetic construct
  source 1..59: mol_type = other DNA
gtgactggagttcagacgtgtgctcttccgatcttctgcaaatgaacagcctgagagcc

SEQ ID NO: 95 - DNA, 59 bp, synthetic construct
  source 1..59: mol_type = other DNA
gtgactggagttcagacgtgtgctcttccgatctgagctctgtgaccgccgcggacacg

SEQ ID NO: 96 - DNA, 59 bp, synthetic construct
  source 1..59: mol_type = other DNA
gtgactggagttcagacgtgtgctcttccgatctcagcaccgcctacctgcagtggagc

SEQ ID NO: 97 - DNA, 59 bp, synthetic construct
  source 1..59: mol_type = other DNA
gtgactggagttcagacgtgtgctcttccgatctgttctccctgcagctgaactctgtg

SEQ ID NO: 98 - DNA, 58 bp, synthetic construct
  source 1..58: mol_type = other DNA
gtgactggagttcagacgtgtgctcttccgatctcagcacggcatatctgcagatcag

SEQ ID NO: 99 - DNA, 35 bp, synthetic construct
  source 1..35: mol_type = other DNA
nantnancntntncntnanatgggaaagagtgtcc

SEQ ID NO: 100 - DNA, 33 bp, synthetic construct
  source 1..33: mol_type = other DNA
acactctttccctacacgacgctcttccgatct

(19) United States
(12) Patent Application Publication
(10) Pub. No.: US 2025/0361550 A1
(43) Pub. Date: Nov. 27, 2025

(54) NUCLEIC ACID AMPLIFICATION

(71) Applicant: SIMSEN DIAGNOSTICS AB, Göteborg (SE)

(72) Inventors: Anders STÅHLBERG, Kållered (SE); Gustav JOHANSSON, Västra Frölunda (SE); Daniel ANDERSSON, Göteborg (SE)

(21) Appl. No.: 19/103,710

(22) PCT Filed: Aug. 5, 2023
(86) PCT No.: PCT/SE2023/050782

§ 371 Date: Feb. 13, 2025

(30) Foreign Application Priority Data
Aug. 15, 2022 (SE) 2250964-0

(51) Int. Cl. C12Q 1/6853; C12Q 1/6851

(52) U.S. Cl. CPC C12Q 1/6853; C12Q 1/6851

(57) ABSTRACT

The present invention determines nucleic acid amplification by contacting a sample comprising a target nucleic acid molecule (1) with forward and reverse primers (10, 20) and amplifying the target nucleic acid molecule (1) by performing PCR pre-amplification to form barcoded PCR products (50). The barcoded PCR products (50) are amplified using adapter-specific forward and reverse primers (30, 40) in a PCR 5 application into amplified barcoded PCR products (60). The forward and/or reverse primers (10, 20) are hairpin barcode primers comprising distributed UMIs (14, 24) protected at least partly inside hairpin loops.

### TECHNICAL FIELD

The present embodiments generally relate to nucleic acid amplification and in particular to nucleic acid amplification using barcoded primers.

### BACKGROUND

Massive parallel sequencing enables a growing number of clinical and basic research applications within many diverse areas, including diagnostics, treatment stratification, drug discovery, forensics, evolutionary studies and environmental DNA testing. Essentially, any type of biological sample can be analyzed, comprising complex samples matrices, such as tissues, body fluids and environmental samples, highly variable sample sizes, ranging from individual molecules to billions of cells, and DNA with different integrity, such intact DNA extracted from living cells to highly fragmented DNA that are typical for tissue fixation, body fluids and forensics samples. Furthermore, numerous approaches exist to analyze anything from a limited number of targeted gene sequences to entire genomes. Conventional sequencing has the sensitivity to detect Variant Allele Frequencies (VAFs) down to 1-5%. This is not adequate for several emerging applications, such as circulating tumor DNA analysis, requiring reliable detection of VAFs<0.1%, sometimes even individual molecules. To overcome this issue, Unique Molecular Identifiers (UMIs), also known as molecular barcodes, are introduced in library construction to enable technical noise reduction in the downstream bioinformatical data analysis. The UMI, typically consisting of an 8-12 nucleotides long and randomized sequence, is added to target DNA using either PCR or hybridization capture-based approaches. Thus, all sequence reads with identical UMI can be bioinformatically traced back to the same template DNA molecule, generating consensus reads that enable polymerase-induced error correction. However, while superior quality of sequencing data is provided by UMIs, most experimental protocols are complicated and includes multi-step protocols. The primary experimental limitation is that the random sequence of UMIs is prone to generate non-specific PCR products that interfere with library construction, especially in PCR-based approaches.

U.S. Pat. No. 10,557,134 discloses approaches for improved detection, identification, and/or quantification of target nucleic acids. These approaches provide a means of detecting, identifying, and/or quantifying rare target nucleic acid molecules, including deoxyribonucleic acid (DNA) and ribonucleic acid (RNA) molecules, from the same sample, and in the same reaction, by using hairpin barcode primers to incorporate unique barcodes into target nucleic acids in a polymerase chain reaction (PCR) pre-amplification step.

U.S. Pat. No. 9,506,119 and U.S. Publication No. 2014/0255929 disclose the use of sequence tags to improve sequence determination of amplicons of related sequences, particularly large and complex amplicons, such as those comprising recombined nucleic acids encoding immune receptor molecules. Sequence reads having the same sequence tags are aligned after which final base calls are determined from an average base call from sequence read base calls at each position. Sequence reads comprising series of incorporation signals are aligned by common sequence tags and base calls in homopolymer regions are made as a function incorporation signal values at each flow position.

### SUMMARY

It is a general objective to provide a method for amplifying nucleic acid molecules in a sample.

This and other objectives are met by embodiments as disclosed herein.

The present invention is defined in the independent claims. Further embodiments are defined in the dependent claims.

An aspect of the invention relates to a method of amplifying a target nucleic acid molecule in a sample.

The method comprises contacting a sample comprising a target nucleic acid molecule with a forward primer and a reverse primer. The forward primer comprises, from a 5′ end to a 3′ end, an adapter sequence and a target-specific sequence and the reverse primer comprises, from a 5′ end to a 3′ end, an adapter sequence and a target-specific sequence. The forward primer is a hairpin barcode forward primer and/or the reverse primer is a hairpin barcode reverse primer. The hairpin barcode forward primer comprises, from the 5′ end to the 3′ end, the adapter sequence, a unique molecular identifier (UMI) sequence comprising a structured UMI, and the target-specific sequence complementary to a portion of the target nucleic acid molecule. The hairpin barcode reverse primer comprises, from the 5′ end to the 3′ end, the adapter sequence, a UMI sequence comprising a structured UMI, and the target-specific sequence complementary to a portion of the target nucleic acid molecule. At least a portion of the adapter sequence of the hairpin barcode forward primer and/or the hairpin barcode reverse primer is complementary to at least a portion of the UMI sequence of the hairpin barcode forward primer and/or the hairpin barcode reverse primer. The adapter sequence and the UMI sequence are configured to hybridize to each other at or under a closed annealing temperature and not hybridize to each other at or above an open annealing temperature. The method also comprises amplifying the target nucleic acid molecule by performing polymerase chain reaction (PCR) pre-amplification of the target nucleic acid molecule to form a plurality of barcoded PCR products. The PCR pre-amplification has an annealing temperature equal to or less than the closed annealing temperature of the hairpin barcode forward primer and/or the hairpin barcode reverse primer. The method further comprises contacting the plurality of barcoded PCR products with an adapter-specific forward primer and an adapter-specific reverse primer and amplifying the barcoded PCR products by performing PCR amplification on the barcoded PCR products to form amplified barcoded PCR products. At least a portion of cycles of the PCR amplification has an annealing temperature equal to or greater than the open annealing temperature of the hairpin barcode forward primer and/or the hairpin barcode reverse primer.

Another aspect of the invention relates to a method for quantifying nucleic acid molecules. The method comprises contacting a sample comprising nucleic acid molecules with P forward primers and Q reverse primers. P is an integer equal to or larger than one and Q is an integer equal to or larger than one. The P forward primers comprise, from a 5′ end to a 3′ end, an adapter sequence and a target-specific sequence and the Q reverse primers comprise, from a 5′ end to a 3′ end, an adapter sequence and a target-specific sequence. The P forward are P hairpin barcode forward primers and/or the Q reverse primers are Q hairpin barcode reverse primers. Each hairpin barcode forward primer comprises, from the 5′ end to the 3′ end, the adapter sequence, a UMI sequence comprising a structured UMI, and the target-specific sequence complementary to a respective portion of a nucleic acid molecule. Each hairpin barcode reverse primer comprises, from the 5′ end to the 3′ end, the adapter sequence, a UMI sequence comprising a structured UMI, and the target-specific sequence complementary to a respective portion of a nucleic acid molecule. At least a portion of the adapter sequence of the hairpin barcode forward primer and/or the hairpin barcode reverse primer is complementary to at least a portion of the UMI sequence of the hairpin barcode forward primer and/or the hairpin barcode reverse primer. The adapter sequence and the UMI sequence are configured to hybridize to each other at or under a closed annealing temperature and not hybridize to each other at or above an open annealing temperature. The method also comprises amplifying the nucleic acid molecules by performing PCR pre-amplification of the nucleic acid molecules to form a plurality of barcoded PCR products. The PCR pre-amplification has an annealing temperature equal to or less than the closed annealing temperature of the hairpin barcode forward primers and/or the hairpin barcode reverse primers. The method further comprises contacting the plurality of barcoded PCR products with an adapter-specific forward primer and an adapter-specific reverse primer and amplifying the barcoded PCR products by performing PCR amplification on the barcoded PCR products to form a library of amplified barcoded PCR products. At least a portion of cycles of the PCR amplification has an annealing temperature equal to or greater than the open annealing temperature of the hairpin barcode forward primers and/or the hairpin barcode reverse primers. The method additionally comprises sequencing at least a respective portion of the amplified barcoded PCR products to form respective sequence reads comprising the UMI(s) and nucleic acid molecule sequence(s). The method also comprises demultiplexing the sequence reads based on nucleic acid sequences of the UMIs and mapping the demultiplexed sequence reads to respective known nucleic acid molecule sequences based on nucleic acid sequences of the nucleic acid molecule sequence(s). The method further comprises quantifying unique nucleic acid molecules based on the demultiplexed and mapped sequence reads.

The present invention provides an amplification method solving issues with non-uniform amplification of nucleic acid molecules and polymerase-induced errors. The invention can be applied to various sample types, including enriched and non-enriched cell populations. The method is simple to conduct, does not require any target nucleic acid molecule capture and provides quantitative information of amplified nucleic acid molecules.

### BRIEF DESCRIPTION OF THE DRAWINGS

The embodiments, together with further objects and advantages thereof, may best be understood by making reference to the following description taken together with the accompanying drawings, in which:

FIG. 1. Flow chart illustrating a method of amplifying a target nucleic acid molecule according to an embodiment.

FIG. 2. Flow chart illustrating a method for quantifying nucleic acid molecules according to an embodiment.

FIG. 3. Schematic illustrations of primers (A, B, C) and amplification steps (A) that can be used in a method of amplifying a target nucleic acid molecule according to various embodiments.

FIG. 4. Schematic illustration of primers (A, B, C) that can be used in a method of amplifying a target nucleic acid molecule according to various embodiments.

FIG. 5. Illustration of different hairpin barcode primer structures. (A) Prior art hairpin barcode primer. (B) Modified hairpin barcode primer with barcode distributed in hairpin loop. (C) Double-loop hairpin barcode primer with barcode distributed in the hairpin loops. (D) Integrated barcode with barcode along stem region.

FIG. 6. Digital sequencing using SiMSen-Seq and structured UMIs. (A) Schematic structure and function of barcode primers with stem-loop protected UMI. Stem opening and closing is temperature dependent. Tm, melting temperature. (B) Overview of the SiMSen-Seq workflow. (C) Sequence design of forward barcoding primer. Different primer elements are indicated by color and name. The stem is stabilized by two nucleotide pairs (GG- and CC stem stabilizers) and destabilized by two nucleotides (AT stem destabilizer). The AT stem destabilizer prohibits the UMI from extending the stem. (D) Design of structured UMIs. The sequence contexts of reference UMI and 14 structured UMIs (A-N) are shown. Designs B and C lack the AT stem destabilizer. Nucleotide N represents any nucleotide type; nucleotide S represents cytosine or guanine; nucleotide W represents adenine or thymine.

FIG. 7. Assay performance using different structured UMIs. (A) Assay performance using quantitative PCR. The relative specificity is indicated as ΔCq, which is the difference in cycle of quantification values between samples with (full lines) and without (hatched lines) DNA. The figure shows the amplification curves of a representative assay (TP53_A, UMI design F), n=3. (B) Relative specificity using quantitative PCR. Data for all structured UMIs (A-N) and the reference UMI are shown for six assays. Mean value for each assay is shown, n=3. The mean of all assays is indicated by a bar for each UMI design. Data are scaled to the reference UMI with a mean value of zero. (C) Assay performance using capillary gel electrophoresis. The electropherograms exemplify representative libraries for a representative assay (TP53_A) using reference UMI and UMI design (J), respectively, n=1. The specific library product is indicated with a bar. (D) Library yield determined with capillary gel electrophoresis. The percentage of correctly formed library relative all generated PCR product is shown for six assays with mean indicated by a bar, n=1. (E) Performance ranking of structured UMIs. The total ranking sum of all the different structured UMIs based on their relative performance using quantitative PCR and capillary gel electrophoresis data. The order of UMI designs in subfigures B and D are based on their final ranks.

FIG. 8. Validation of improved assay performance using structured UMIs. (A) Relative specificity using quantitative PCR. Data for UMI design (F) and the reference UMI are shown for 32 assays. Mean ΔCq value of samples with 20 ng DNA (PTC) compared with water (NTC) is shown for each assay and UMI design, n=3. Box plot of all data is shown to the right. ***p≤0.001, paired Student's t-test. (B) Specificity in correct library products using capillary gel electrophoresis. Data for UMI design (F) and the reference UMI are shown for 32 assays, n=1. Box plot of all data is shown to the right. ***p≤0.001, paired Student's t-test. (C) Melting temperature of forward barcoding primer. Data for UMI design (F) and the reference UMI are shown for 32 assays. Mean melting temperature is shown for each assay and UMI design, n=2. Box plot of all data is shown to the right. ***p≤0.001, paired Student's t-test.

FIG. 9. Evaluation of 12 different 3-plexes. (A) Relative specificity using quantitative PCR. Twelve 3-plexes were analyzed using UMI design (F) and reference UMI. Mean value for each assay is shown, n=3. Box plot of all data is shown to the right. ***p≤0.001, paired Student's t-test. (B) Specificity in correct library products using capillary gel electrophoresis. Twelve 3-plexes were analyzed using UMI design (F) and reference UMI. Mean value for each assay is shown, n=3. Box plot of all data is shown to the right. ***p≤0.001, paired Student's t-test. (C) On-target amplification assessed by digital sequencing. Number of consensus reads are shown for each assay in respective 3-plex using UMI design (F) and reference UMI. Mean value for each assay is shown, n=3. Box plot of all data is shown to the right. ***p≤0.001, paired Student's t-test.

FIG. 10. Performance of 16-plex. The coverage calculated as consensus reads is shown for all individual assays. Mean coverage is shown for each assay, n=3. Box plot of all data is shown to the right. *p≤0.05, paired Student's t-test.

FIG. 11. Library yield determined with capillary gel electrophoresis. The electropherograms for UMI design (F) and reference UMI are shown, n=1.

FIG. 12. Melting curve analysis. (A) Melting curves shown for SiMSen-Seq forward primers with UMI design (F) and reference UMI in red and black, respectively. The dotted vertical lines indicate melting temperatures (Tm) of respective SiMSen-Seq primer stem-loop structure. n=3. (B) Melting temperatures of the stem structures for all structured UMIs (A-N) and reference UMI is shown for six assays with mean indicated by a bar. Mean melting temperature is shown for each assay and UMI design, n=3. (C) Melting temperature versus nucleotide length of loop structure. The spearman correlation coefficient (p) was −0.86, p<0.001. The linear fit is to guide the eye.

FIG. 13. Comparison of assay performance. The coverage of individual assays in 3-plexes is correlated to (A-B) the performance of single-plexes assessed with quantitative PCR and (C) the performance of single-plexes assessed with capillary gel electrophoresis. Cq (PTC) is the cycle of quantification value for samples with 20 ng DNA. The spearman correlation coefficient (p) is calculated for UMI design (F) (n=30), reference UMI (n=30) and all together (n=60). The linear fit is to guide the eye.

FIG. 14. Dynamic range of 3-plex 6 and UMI design (F). Linear regression is shown for each assay and consensus reads are used as coverage, n=3.

FIG. 15. Comparison of assay performance. The coverage of individual assays in 16-plexes is correlated to (A-B) the performance of single-plexes assessed with quantitative PCR and (C) the performance of single-plexes assessed with capillary gel electrophoresis. Cq (PTC) is the cycle of quantification value for samples with 20 ng DNA. The spearman correlation coefficient (p) is calculated for UMI design (F) (n=15), reference UMI (n=15) and all together (n=30). The linear fit is to guide the eye. (D) Coverages of individual assays in 3 plexes versus 16-plex, n=16 and n=32 for all assays.

FIG. 16. Evaluation of background error rates. (A) Sequencing error rate for 3-plexes. The error rate was calculated as the total number of non-reference alleles per nucleotide position divided by coverage for each nucleotide position. Then the average of all nucleotide positions was calculated per amplicon. Mean±SD is shown, n=3. Nucleotide positions corresponding to known single nucleotide positions (rs788018, rs4685 and rs1800372) were omitted for background noise visualization purposes. Box plot of all data is shown to the right. Paired Student's t-test. n.s., non-significant. (B) Sequencing error rate for 16-plex. Mean±SD is shown, n=3. Box plot of all data is shown to the right. Paired Student's t-test. n.s., non-significant.

FIG. 17. Illustration of 6 TCR repertoire sequencing. (A) Schematic overview of the unrearranged TCR δ (TRD) locus, arrows show transcription direction. TRD variable 4 (TRDV4) to TRDV8 are shared between the TCR α (TRA) and TRD locus (see Table 1). (B) Amplification consists of two rounds of PCR. In the first barcoding PCR, target primers bind to the V and J genes, generating specific PCR products with UMIs and flanked adapter sequences. In the second adapter PCR, barcoded DNA is amplified with ILLUMINA® adapter primers. (C) The experimental workflow for VDJ-sequencing. (D) Design of synthetic reference molecules used for assay validation. The 32 gBlock molecules contain a TRDV segment consisting of the 97-163 last base pairs of the TRDV gene, and a 48-59 base pairs long TRD joining (TRDJ) segment containing the complete TRDJ gene. An internal unique template specific sequence for each primer pair combination is inserted between the TRDV and TRDJ segments. All synthetic gBlock molecules are also flanked by a non-amplified general sequence. All synthetic reference molecules are shown in Table 1.

FIG. 18. Illustration of amount of off-target sequence reads. Mean value for each tri-plex is shown, n=3. Box plots of all mean values are shown to the right. **p≤0.01, paired Student's t-test.

FIG. 19. Illustration of mean off-target reads for all individual assays, n=3. ***p≤0.001, unpaired Student's t-test.

FIG. 20. Illustration of sensitivity to detect mutated DNA molecules.

### DETAILED DESCRIPTION

The present embodiments generally relate to nucleic acid amplification and in particular to nucleic acid amplification using barcoded primers.

Unique molecular identifiers (UMIs), or molecular barcodes (MBCs), are short sequences or tags added to DNA fragments in some next-generation sequencing (NGS) library preparation protocols to identify the input DNA molecule. These UMIs are added before the actual PCR amplification, and can be used to reduce errors and quantitative bias introduced by the amplification. UMIs can be added to DNA molecules by either ligation- or PCR-based approaches. Ligation-based UMI approaches require that target DNA is captured before the analysis, otherwise, all genomic DNA will be analyzed. Another limitation is that molecules are lost due to limited ligation efficiency. In comparison, PCR-based UMI approaches are simpler since no capture step is needed. PCR-based methods are potentially also more sensitive since they do not suffer from ineffective capture and ligation steps. However, introduction of UMIs into PCR primers may cause massive formation of non-specific PCR products caused by the random nucleotide sequence of UMIs.

This problem is solved according to the present invention by shielding the UMIs in secondary structures in hairpin barcode forward primers and/or hairpin barcode reverse primers. Hence, in order to minimize the formation of non-specific PCR products, the UMI is protected in the hairpin barcode primer, such as at least partly inside a hairpin loop, which opens and closes its secondary structure in a temperature-dependent manner.

The hairpin barcode primers of the present invention, in addition, comprise structured UMI sequences protected at least partly inside the hairpin loops. Experimental data as presented herein indicates that using structured UMI sequences, such as by distributing the UMI parts at least partly inside one or more hairpin loops, significantly improves the purity of the barcoded PCR products as compared to using hairpin barcode primers with a non-structured UMI sequence with consecutive random nucleotides. Furthermore, the purity of the library as obtained following PCR-based amplification is considerably higher as compared to using non-structured UMI sequences in hairpin barcode primers.

The hairpin barcode primers of the present invention therefore comprises so-called structured UMIs, i.e., UMIs comprising predefined or degenerated nucleotides at specific positions within the random nucleotides of the structured UMIs. As is shown herein, such structured UMIs reduce the formation of non-specific amplification (PCR) products as compared to traditional UMIs only consisting of a sequence of random nucleotides. The presence of predefined or degenerated nucleotides within the structured UMIs reduce the possibility of forming stable internal structures and dimers, which could cause the formation of non-specific PCR products. Furthermore, the structured UMIs of the invention resulted in less off-target reads, which thereby saves sequencing capacity. Accordingly, a library of amplified nucleic acid molecules generated by the invention has a higher quality and is thereby more suitable for sequencing as compared to using primers with traditional non-structured UMIs.

Accordingly, the usage of hairpin barcode primers of the invention enables an improved amplification of a target nucleic acid sequence in a sample.

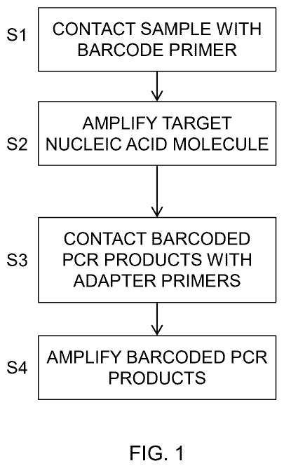

An aspect of the invention relates to a method of amplifying a target nucleic acid molecule in a sample, see FIGS. 1 and 3A to 3C and 4A to 4C. The method comprises contacting, in step S1, a sample comprising a target nucleic acid molecule 1 with a forward primer 10 and a reverse primers 20.

The forward primer 10 comprises, from a 5′ end 10A to a 3′ end 10B, an adapter sequence 11 and a target-specific sequence 16. Correspondingly, the reverse primer 20 comprises, from a 5′ end 20A to a 3′ end 20B, an adapter sequence 21 and a target-specific sequence 26.

According to the invention, the forward primer 10 is a hairpin barcode forward primer 10 and/or the reverse primer 20 is a hairpin barcode reverse primer 20. In such a case, the hairpin barcode forward primer 10 comprises, from the 5′ end 10A to the 3′ end 10B, the adapter sequence 11, a unique molecular identifier (UMI) sequence 17 comprising a structured UMI 14, and the target-specific sequence 16 complementary to a portion 2 of the target nucleic acid molecule 1. Correspondingly, the hairpin barcode reverse primer 20 comprises, from the 5′ end 20A to the 3′ end 20B, the adapter sequence 21, a UMI sequence 27 comprising a structured UMI 24, and the target-specific sequence 26 complementary to a portion 3 of the target nucleic acid molecule 1. At least a portion 12, 22 of the adapter sequence 11, 21 of the hairpin barcode forward primer 10 and/or the hairpin barcode reverse primer 20 is complementary to at least a portion 15, 25 of the UMI sequence 17, 27 of the hairpin barcode forward primer 10 and/or the hairpin barcode reverse primer 20. The adapter sequence 11, 21 and the UMI sequence 17, 27 are configured to hybridize to each other at or under a closed annealing temperature and not hybridize to each other at or above an open annealing temperature.

According to the invention, the UMI sequence 17 of the hairpin barcode forward primer 10 comprises a structured UMI 14. Correspondingly, the UMI sequence 27 of the hairpin barcode reverse primer 20 comprises a structured 24. Structured UMI 14, 24 as used herein means that the UMI 14, 24 does not only consist of random nucleotides. In clear contrast, the structured UMI 14, 24 also comprises one or more parts or sequences having predefined nucleotides, i.e., not random nucleotides, and/or degenerated nucleotides, i.e., not fully random nucleotides but restricted in terms of excluding at least one nucleotide.

In the former case, the structured UMI 14, 24 comprises at least one, preferably multiple UMI parts 14A, 14B, 24A, 24B separated by a respective predefined nucleic acid sequence 14C, 24C. This predefined nucleic acid sequence 14C, 24C thereby has a fixed or predefined set of one or more nucleotides, whereas the nucleotide(s) in the UMI part(s) 14A, 14B, 24A, 24B are randomly generated from the set of nucleotides, such as adenine (A), cytosine (C), guanine (G) and thymine (T) for a DNA-based hairpin barcode primer 10, 20 or A, C, G and uracil (U) for an RNA-based barcode primer 10, 20.

In the latter case, one or more degenerated nucleotides are not fully randomized, i.e., cannot be selected among the full set of A, C, G and T or A, C, G and U. Examples of such degenerated nucleotides are weak (W), i.e., A or T; strong (S), i.e., C or G; amino (M), i.e., A or C; keto (K), i.e., G or T; purine (R), i.e., A or G; pyrimidine (Y), i.e., C or T; not A (B), i.e., C, G or T; not C (D), i.e., A, G or T; not G (H), i.e., A, C or T; or not T (V), i.e., A, C or G.

The method as shown in FIG. 1 also comprises amplifying, in step S2, the target nucleic acid molecule 1 by performing polymerase chain reaction (PCR) pre-amplification of the target nucleic acid molecule 1 to form a plurality of barcoded PCR products 50. The PCR pre-amplification as performed in step S2 has an annealing temperature equal to or less than the closed annealing temperature of the hairpin barcode forward primer 10 and/or the hairpin barcode reverse primers 20.

The method further comprises contacting, in step S3, the plurality of barcoded PCR products 50 with an adapter-specific forward primer 30 and an adapter-specific reverse primer 40 and amplifying, in step S4, the barcoded PCR products 50 by performing PCR amplification on the barcoded PCR products 50 to form amplified barcoded PCR products 60. In an embodiment, at least a portion of cycles of the PCR amplification has an annealing temperature greater than or equal to the open annealing temperature of the hairpin barcode forward primers 10 and/or the hairpin barcode reverse primer 20.

The amplifying method of the invention as shown in FIG. 1 could be used to amplify any target nucleic acid molecule in any sample comprising such nucleic acid molecules. The amplifying method is in particular suitable for amplifying nucleic acid molecules in complex samples comprising a mixture of a plurality of different nucleic acid molecules.

Illustrative, but non-limiting, examples of such samples are biological samples comprising nucleic acid molecules, such as cellular samples, tissue samples, or body fluid samples. Alternatively, the sample could be a sample comprising nucleic acid molecules purified or isolated from such a cellular, tissue, or body fluid sample. The sample can then be an animal sample, such a mammalian sample, and preferably a human sample. Particular examples of samples include plasma sample, serum sample, sputum sample, skin sample, spinal fluid sample, lymph fluid sample, synovial fluid sample, urine sample, tear sample, blood cells sample, tissue sample from an organ or a tumor, whole blood sample, bone marrow sample, amniotic fluid sample, hair sample, semen sample, anal secretions sample, vaginal secretions sample, perspiration sample, saliva sample, and buccal swabs sample. The sample could alternatively be a sample obtained from an in vitro cell culture. Other examples include microbial or microorganism samples, such as bacterial samples, virus samples, yeast samples, etc. Also environmental samples could be used including, but not limited to, water samples, soil samples, waste samples, etc.

The target nucleic acid molecule could be an RNA molecule or DNA molecule, including a complementary DNA (cDNA) molecule and modified RNA and DNA molecules, such containing nucleic acid analogues, preferably a DNA molecule.

The amplifying method of the invention can be used in various applications. An illustrative example of such an application is detection of cell-free DNA (cfDNA) in liquid biopsies. Such applications offer great potential for use in non-invasive prenatal testing and as a cancer biomarker. Fetal and tumor DNA fractions, however, can be extremely low in such liquid biopsy samples and ultra-sensitive methods are required for their detection. Hairpin barcode primer with a structured UMI can then be used to generate consensus reads for each original DNA molecule in the liquid biopsy sample, reduce background sequencing noise and allow detection of variant alleles below 0.1% frequency in clonal cell line DNA and in cell-free plasma DNA. The amplifying method of the invention can therefore be used to enable sensitive mutation detection in liquid biopsies. For instance, hairpin barcode primers with structured UMIs can be used instead of the barcode primers disclosed in Nucleic Acids Research (2016) 44(11): e105.

The amplifying method of the invention can also be used in detection of extremely rare variant alleles within a complex mixture of DNA molecules, such as the detection of circulating tumor DNA in the plasma of cancer patients. Barcoding of DNA template molecules early in NGS library construction provides a way to identify and bioinformatically remove polymerase errors that otherwise make detection of these rare variants very difficult. Hairpin barcode primers with structured UMIs can be used to generate targeted barcoded libraries with minimal DNA input, flexible target selection and a very simple, short library construction protocol. For instance, hairpin barcode primers with structured UMIs can be used instead of the barcode primers disclosed in Nature Protocols (2017) 12(4): 664-682.

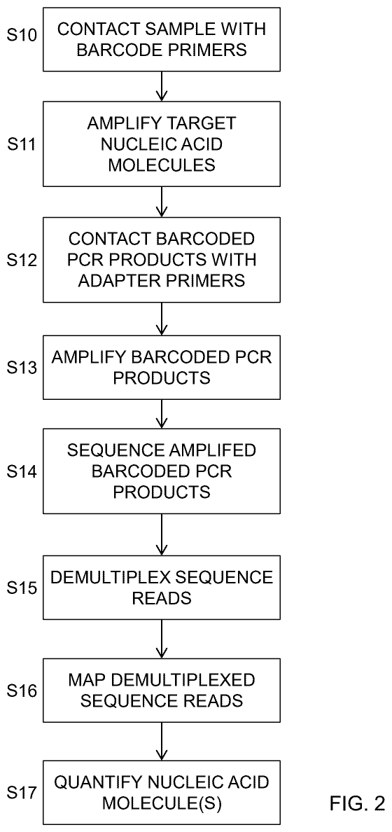

Another aspect of the invention relates to a method for quantifying nucleic acid molecules, see FIGS. 2, 3A to 3C and 4A to 4C. The method comprises contacting, in step S10, a sample comprising nucleic acid molecules 1 with P forward primers 10 and Q reverse primers 20.

In an embodiment, P is an integer equal to or larger than one and Q is an integer equal to or larger than one. The P forward primers 10 comprise, from a 5′ end 10A to a 3′ end 10B, an adapter sequence 11 and a target-specific sequence 16. Correspondingly, the Q reverse primers 20 comprise, from a 5′ end 20A to a 3′ end 20B, an adapter sequence 21 and a target-specific sequence 26.

In an embodiment, the P forward primers 10 are P hairpin barcode forward primers 10 and/or the Q reverse primers 20 are Q hairpin barcode reverse primers 20. In such an embodiment, each hairpin barcode forward primer 10 comprises, from the 5′ end 10A to the 3′ end 10B, the adapter sequence 11, a UMI sequence 17 comprising a structured UMI 14, and the target-specific sequence 16 complementary to a respective portion 2 of a nucleic acid molecule 1. Correspondingly, each hairpin barcode reverse primer 20 comprises, from the 5′ end 20A to the 3′ end 20B, the adapter sequence 23, a UMI sequence 27 comprising a structured UMI 24, and the target-specific sequence 26 complementary to a respective portion 3 of a nucleic acid molecule 1. At least a portion 12, 22 of the adapter sequence 11, 21 of the hairpin barcode forward primer 10 and/or the hairpin barcode reverse primer 20 is complementary to at least a portion 15, 25 of the UMI sequence 17, 27 of the hairpin barcode forward primer 10 and/or the hairpin barcode reverse primer 20. The adapter sequence 11, 21 and the UMI sequence 17, 27 are configured to hybridize to each other at or under a closed annealing temperature and not hybridize to each other at or above an open annealing temperature.

The method as shown in FIG. 2 also comprises amplifying, in step S11, the nucleic acid molecules 1 by performing PCR pre-amplification of the nucleic acid molecules 1 to form a plurality of barcoded PCR products 50. The PCR pre-amplification as performed in step S11 has an annealing temperature equal to or less than the closed annealing temperature of the hairpin barcode forward primers 10 and/or the hairpin barcode reverse primers 20.

The method further comprises contacting, in step S12, the plurality of barcoded PCR products 50 with an adapter-specific forward primer 30 and an adapter-specific reverse primer 40 and amplifying, in step S13, the barcoded PCR products 50 by performing PCR amplification on the barcoded PCR products 50 to form a library of amplified barcoded PCR products 60. In an embodiment, at least a portion of cycles of the PCR amplification has an annealing temperature greater than or equal to the open annealing temperature of the hairpin barcode forward primers 10 and/or the hairpin barcode reverse primers 20. The method also comprises sequencing, in step S14, at least a respective portion of the amplified barcoded PCR products 60 to form respective sequence reads comprising the UMI(s) 64 and nucleic acid molecule sequence(s) 66. The method further comprises demultiplexing, in step S15, the sequence reads based on nucleic acid sequences of the UMIs 64 and mapping, in step S16, the demultiplexed sequence reads to respective known nucleic acid molecule sequences based on nucleic acid sequences of the nucleic acid molecule sequence(s) 66. The method also comprises quantifying, in step S17, unique nucleic acid molecules 1 based on the demultiplexed and mapped sequence reads.

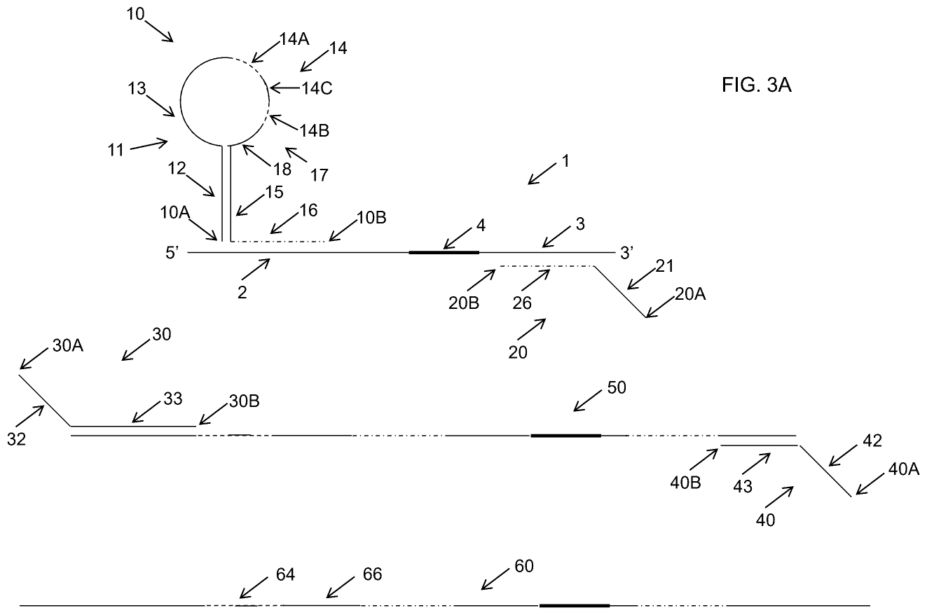

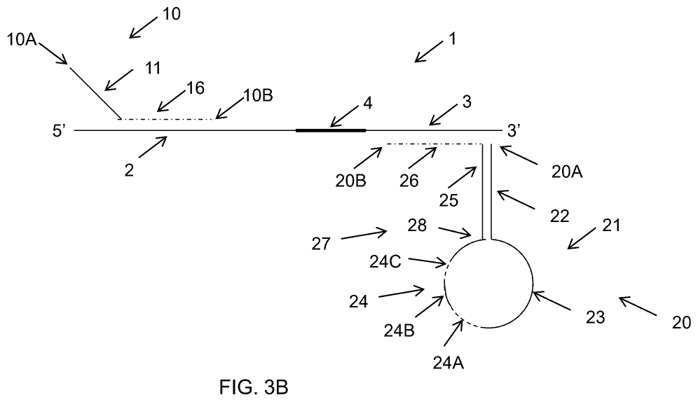

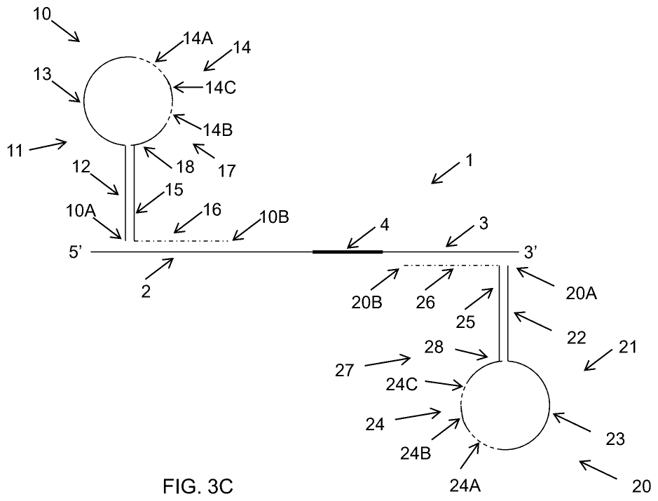

In an embodiment, the forward primer 10 used in FIG. 1 is a hairpin barcode primer 10, whereas the reverse primer 20 does not form any hairpin or loop structure as shown in FIG. 3A. In such an embodiment, step S1 comprises contacting the sample comprising the target nucleic acid molecule 1 with a hairpin barcode forward primer 10 and a reverse primer 20. The hairpin barcode forward primer 10 then comprises, from the 5′ end 10A to the 3′ end 10B, the adapter sequence 11, the UMI sequence 17, and the target-specific sequence 16. The at least one reverse primer 20 comprises, from the 5′ end 20A to the 3′ end 20B, the adapter sequence 21 and the target-specific sequence 26.

Correspondingly, in an embodiment, the P forwards primers 10 used in FIG. 2 are P hairpin barcode forward primers 10, whereas the Q reverse primers 20 do not form any hairpin or loop structure as shown in FIG. 3A. In such an embodiment, step S10 comprises contacting the sample comprising nucleic acid molecules 1 with a set of P>1 hairpin barcode forward primers 10 and at least one reverse primer 20. Each hairpin barcode forward primer 10 of the set comprises, from the 5′ end 10A to the 3′ end 10B, the adapter sequence 11, the UMI sequence 17, and the target-specific sequence 16 complementary to a respective portion 2 of a nucleic acid molecule 1. The at least one reverse primer 20 comprises, from the 5′ end 20A to the 3′ end 20B, the adapter sequence 21 and the target-specific sequence 26.

In an embodiment, all hairpin barcode forward primers 10 comprise the same adapter sequence 11. However, the hairpin barcode forward primers 10 comprise different UMI sequences 17 comprising different structured UMIs 14. The set may comprise hairpin barcode forward primers 10 all having the same target-specific sequence 16 (but different structured UMIs 14), multiple, i.e., at least two, hairpin barcode forward primers 10 having the same target-specific sequence 16 or the hairpin barcode forward primers 10 in the set may have different target-specific sequences 16.

It is, however, also possible to use a mixture of hairpin barcode forward primers 10 that also have different adapter sequences 11. For instance, a first set of hairpin barcode forward primers 10 all have the same adapter sequence 11 but different UMI sequences 17. This first set of hairpin barcode forward primers 10 could have the same or different target-specific sequence 16. This first set of hairpin barcode forward primers 10 is then mixed with at least a second set of hairpin barcode forward primers 10 all having the same adapter sequence 11 but different UMI sequences 17. This second set of hairpin barcode forward primers 10 could have the same or different target-specific sequence 16. The adapter sequence 11 of the hairpin barcode forward primers 10 in the second set is, though, different from the adapter sequence 11 of the hairpin barcode forward primers 10 of the first set.

In an embodiment, a mixture of hairpin barcode forward primers 10 that have different designs of UMI sequences 17, such as different designs of the structured UMI 14, could be used. For instance, the length of the structured UMI 14 in terms of number of nucleotide sequences and/or the particular design of the structured UMI 14 in terms of distribution of random nucleotides and predefined nucleotides could differ between different hairpin barcode forward primers 10. Such a mixture of different hairpin barcode forward primers 10 could be useful in case the sample comprises a mixture of different target nucleic acid molecules, which may be present in different in different amounts or levels in the sample, such as few copy number versus high copy number.

An illustrative, but non-limiting, example of a hairpin barcode forward primer 10 that can be used in accordance with the embodiments have the following sequence (SEQ ID NO: 1):

5′-GGACACTCTTTCCCTACACGACGCTCTTCCGATCTNANTNANCNTN
TNCNTNANATGGGAAAGAGTGTCC-target-specific
sequence-3′

In this illustrative example, the adapter sequence 11 comprises, preferably consists of, the nucleotide sequence GGACACTCTTTCCCTACACGACGCTCTTCCGATCT (SEQ ID NO: 4) that comprises a part (GGACACTCTTTCCC SEQ ID NO: 2), denoted 5′ stem sequence 12 herein, which is complementary to and capable of hybridizing to a part (GGGAAAGAGTGTCC (SEQ ID NO: 3), denoted 3′ stem sequence 15 herein, of the UMI sequence 17 NANTNANCNTNTNCNTNANATGGGAAAGAGTGTCC (SEQ ID NO: 99). The UMI sequence 17 comprises a structured UMI 14 represented, in this example, by NANTNANCNTNTNCNTNAN (SEQ ID NO: 90) and having alternating random nucleotides (N) 14A, 14B and predefined nucleotides (A, T or C) 14C. The adapter sequence 11 of the hairpin barcode forward primer 10 additionally comprises, in this example, a nucleotide sequence 13 that is not complementary to the UMI sequence 17. This non-complementary sequence 13 may be positioned between the 5′ stem sequence 12 and the UMI sequence 17. For instance, a non-complementary sequence 13 of ACACTCTTTCCCTACACGACGCTCTTCCGATCT (SEQ ID NO: 100) could be used in the above illustrated example.

In an embodiment, a single or common reverse primer 20 is used together with the P hairpin barcode forward primers 10 in FIG. 2. The common reverse primer 20 comprises a target-specific sequence 26 that is complementary to sequence or region 3 of the nucleic acid molecule 1 that is common for the different nucleic acid molecules.

In another embodiment, multiple different reverse primers 20 are used together with the P hairpin barcode forward primers 10 in FIG. 2. Each reverse primer 20 then preferably comprises a respective target-specific sequence 26 that is complementary to a respective sequence or region 3 of the nucleic acid molecule 1 that is specific for a given nucleic acid molecule or specific for a given group of nucleic acid molecules, i.e., is not common for all different nucleic acid molecules.

In an embodiment, all reverse primers 20 comprise the same adapter sequence 26. However, the reverse primers 20 may comprise different target-specific sequences 26.

An illustrative, but non-limiting, example of reverse primers 20 that can be used in accordance with the embodiments have the following sequence (SEQ ID NO: 5):

5′-GTGACTGGAGTTCAGACGTGTGCTCTTCCGATCT-target-
specific region-3′

In an embodiment, the adapter sequence 11 of the P hairpin barcode forward primers 10 is different from the adapter sequence 21 of the at least one reverse primer 20.

In another embodiment, the reverse primer 20 used in FIG. 1 is a hairpin barcode primer 20, whereas the forward primer 10 does not form any hairpin or loop structure as shown in FIG. 3B. In such an embodiment, step S1 comprises contacting the sample comprising the target nucleic acid molecule 1 with a forward primer 10 and a hairpin barcode reverse primer 20. The hairpin barcode reverse primer 20 then comprises, from the 5′ end 20A to the 3′ end 20B, the adapter sequence 21, the UMI sequence 27, and the target-specific sequence 26. The forward primer 10 comprises, from the 5′ end 10A to the 3′ end 10B, the adapter sequence 11 and the target-specific sequence 16.

Correspondingly, in another embodiment, the Q reverse primers 20 in FIG. 2 are Q hairpin barcode reverse primers 20, whereas the P forward primers 10 do not form any hairpin or loop structure as shown in FIG. 3B. In such an embodiment, step S10 comprises contacting the sample comprising nucleic acid molecules 1 with a set of Q>1 hairpin barcode reverse primers 20 and at least one forward primer 10. Each hairpin barcode reverse primer 20 of the set comprises, from the 5′ end 20A to the 3′ end 20B, the adapter sequence 21, the UMI sequence 27, and the target-specific sequence 26 complementary to a respective portion 3 of a nucleic acid molecule. The at least one forward primer 10 comprises, from the 5′ end 10A to the 3′ end 10B, the adapter sequence 11 and the target-specific sequence 16.

In an embodiment, all Q hairpin barcode reverse primers 20 comprise the same adapter sequence 21. However, the Q hairpin barcode reverse primers 20 comprise different UMI sequences 27 comprising different structured UMIs 24. The set may comprise hairpin barcode reverse primers 20 all having the same target-specific sequence 26 (but different structured UMIs 24), multiple hairpin barcode reverse primers 20 having the same target-specific sequence 26 or the hairpin barcode reverse primers 20 in the set may have different target-specific sequences 26.

It is, however, also possible to use a mixture of hairpin barcode reverse primers 20 that also have different adapter sequences 21. For instance, a first set of hairpin barcode reverse primers 20 all have the same adapter sequence 21 but different UMI sequences 27. This first set of hairpin barcode reverse primers 20 could have the same or different target-specific sequence 26. This first set of hairpin barcode reverse primers 20 is then mixed with at least a second set of hairpin barcode reverse primers 20 all having the same adapter sequence 21 but different UMI sequences 27. This second set of hairpin barcode reverse primers 20 could have the same or different target-specific sequence 26. The adapter sequence 21 of the hairpin barcode reverse primers 20 in the second set is, though, different from the adapter sequence 21 of the hairpin barcode reverse primers 20 of the first set.

In an embodiment, a mixture of hairpin barcode reverse primers 20 that have different designs of UMI sequences 27, such as different designs of the structured UMI 24, could be used. For instance, the length of the structured UMI 24 in terms of number of nucleotide sequences and/or the particular design of the structured UMI 24 in terms of distribution of random nucleotides and predefined nucleotides could differ between different hairpin barcode reverse primers 20. Such a mixture of different hairpin barcode reverse primers 20 could be useful in case the sample comprises a mixture of different target nucleic acid molecules, which may be present in different in different amounts or levels in the sample, such as few copy number versus high copy number.

In an embodiment, a single or common forward primer 10 is used together with the Q hairpin barcode reverse primers 20 in FIG. 2. The common forward primer 10 comprises target-specific sequence 16 that is complementary to sequence or region 2 of the nucleic acid molecule 1 that is common for the different nucleic acid molecules 1.

In another embodiment, multiple different forward primers 10 are used together with the Q hairpin barcode reverse primers 20 in FIG. 2. Each forward primer 10 then preferably comprises a respective target-specific sequence 16 that is complementary to a respective sequence or region 2 of the nucleic acid molecule 1 that is specific for a given nucleic acid molecule or specific for a given group of nucleic acid molecules, i.e., is not common for all different nucleic acid molecules.

In an embodiment, all forward primers 10 comprise the same adapter sequence 11. However, the forward primers 10 may comprise different target-specific sequences 16.

In an embodiment, the adapter sequence 21 of the hairpin barcode reverse primers 20 is different from the adapter sequence 11 of the at least one forward primer 10.

In a further embodiment, both the forward primer 10 and the reverse primer 20 used in FIG. 1 are hairpin barcoded primers as shown in FIG. 3C. In such an embodiment, step S1 comprises contacting the sample comprising the target nucleic acid molecule 1 with a hairpin barcode forward primer 10 and a hairpin barcode reverse primer 20. The hairpin barcode forward primer 10 then comprises, from the 5′ end 10A to the 3′ end 10B, the adapter sequence 11, a UMI sequence 17 comprising a first structured UMI 14, and the target-specific sequence 16 and the hairpin barcode reverse primer 20 comprises, from the 5′ end 20A to the 3′ end 20B, the adapter sequence 21, a UMI sequence 27 comprising a second structured UMI 24, and the target-specific sequence 26.

Correspondingly, in a further embodiment, both the P forward primers 10 and the Q reverse primers 20 used in FIG. 2 are hairpin barcoded primers as shown in FIG. 3C. In such an embodiment, step S10 comprises contacting the sample comprising nucleic acid molecules 1 with a first set of P>1 hairpin barcode forward primers 10 and a second set of Q>1 hairpin barcode reverse primers 20. Each hairpin barcode forward primer 10 of the first set comprises, from the 5′ end 10A to the 3′ end 10B, the adapter sequence 11, a UMI sequence 17 comprising a first structured UMI 14, and the target-specific sequence 16 complementary to a respective portion 2 of a nucleic acid molecule 1 and each hairpin barcode reverse primer 20 of the second set comprises, from the 5′ end 20A to the 3′ end 270B, the adapter sequence 21, a UMI sequence comprising a second structured UMI 24, and the target-specific sequence 26 complementary to a respective portion 3 of a nucleic acid molecule 1.

In an embodiment, all hairpin barcode forward primers 10 of the first set comprise the same adapter sequence 11. However, the P hairpin barcode forward primers 10 comprise different UMI sequences 17 comprising different structured UMIs 14.

In an embodiment, all hairpin barcode reverse primers 20 of the second set comprise the same adapter sequence 21. However, the Q hairpin barcode reverse primers 20 comprise different UMI sequences 17 comprising different structured UMIs 24.

In an embodiment, the adapter sequences 11, 21 comprises a 5′ stem sequence 12, 22 and the UMI sequences 17, 27 comprises a 3′ stem sequence 15, 25. In such an embodiment, the 5′ stem sequence 12 of the P hairpin barcode forward primers 10 is the same as the 5′ stem sequence 22 of the Q hairpin barcode reverse primers 20. In another embodiment, the 5′ stem sequence 12 of the P hairpin barcode forward primers 10 is different from the 5′ stem sequence 22 of the Q hairpin barcode reverse primers 20. Correspondingly, the 3′ stem sequence 15 of the P hairpin barcode forward primers 10 could be the same as or different from the 3′ stem sequence 25 of the Q hairpin barcode reverse primers 20. However, the adapter sequence 11 of the P hairpin barcode forward primers 10 is preferably different from the adapter sequence 21 of the Q hairpin barcode reverse primers 20.

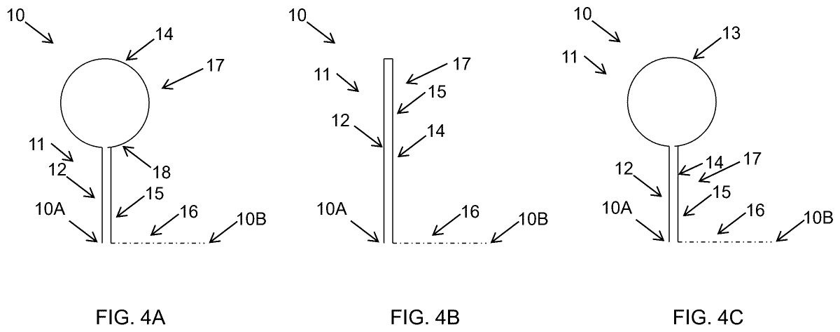

The hairpin barcode forward primer(s) 10 and/or the hairpin barcode reverse primer(s) 20 form a hairpin structure shielding the structured UMI 14, 24, in an embodiment, inside the hairpin loop as shown in FIGS. 3A to 3C, 4A to 4C at a temperature equal to or below the closed annealing temperature. Hence, equal to or below this closed annealing temperature at least a portion 12, 22 of the adapter sequence 11, 21 hybridizes to at least a portion 15, 25 of the UMI sequence 17, 27. Correspondingly, when the temperature increases to or above the open annealing temperature, the least a portion 12, 22 of the adapter sequence 11, 21 no longer hybridizes to the at least a portion 15, 25 of the UMI sequence 17, 27.

The length of the respective portions 12, 22, 15, 25 of the sequences 12, 22, 15, 25 of the hairpin barcode forward primer(s) 10 and/or the hairpin barcode reverse primer(s) 20 that are configured to hybridize to each other define at least partly the closed and open annealing temperatures. Generally, the longer the hybridizing portions of the adapter sequences 11, 21 and the UMI sequences 17, 27, i.e., the higher number of nucleotides in these hybridizing portions, the higher the closed and open annealing temperatures. The closed and open annealing temperatures are not only defined based on the length of the hybridizing portions but also by the particular combination of nucleotides in the hybridizing portions. For instance, hybridizing portions having a higher GC content (G-C and C-G base pairing) have generally a higher temperature of melting (Tm) and thereby a higher closed and opened annealing temperature as compared to hybridizing portions of the same length but lower GC content, i.e., a higher AT content (A-T and T-A base pairing). Also modified nucleotides and nucleic acid analogues, for instance, peptide nucleic acid (PNA), locked nucleic acid (LNA), glycol nucleic acid (GNA), threose nucleic acid (TNA) and Zip nucleic acid (ZNA), present in the adapter sequence 11, 21 and/or the UMI sequence 17, 27 could be used to modify the closed and open annealing temperature. Such modified nucleotides and nucleic acid analogues could then be used to define and tailor the closed and open annealing temperatures of the hairpin barcode forward primers 10 and/or the hairpin barcode reverse primers 20. For instance, LNA generally increases the Tm of two complementary sequences as compared to the naturally occurring nucleotides A, T, G and C. Hence, introducing one or more of LNAs in the adapter sequence 11, 21 and/or the UMI sequence 17, 27 could be used to achieve a target closed or open annealing temperature but using shorter hybridizing portions of the adapter sequences 11, 21 and shorter hybridizing portions of the UMI sequences 17, 27 as compared to when using only naturally occurring nucleotides.

In a particular embodiment, the hybridizing portions of the adapter sequence 11, 21 and of the UMI sequences 17, 27, such as the 5′ stem sequence 12, 22 and the 3′ stem sequence 15, 25, comprises 5-15-nucleotides, preferably 8-15 nucleotides, and more preferably 12-15 nucleotides.

In an embodiment, the hairpin barcode forward primer(s) 10 comprises, from the 5′ end 10A to the 3′ end 10B, the adapter sequence 11, the UMI sequence 17 comprising the structured UMI 14 and a stem sequence 15, and the target-specific sequence 16. Correspondingly, the hairpin barcode reverse primer(s) 20 comprises, from the 5′ end 20A to the 3′ end 20B, the adapter sequence 21, the UMI sequence 27 comprising the structured UMI 24 and a stem sequence 25, and the target-specific sequence 26. In this embodiment, at least a portion 12, 22 of the adapter sequence 11, 21 of the hairpin barcode forward primer 10 and/or the hairpin barcode reverse primer 20 is complementary to at least a portion of the stem sequence 15, 25 of the hairpin barcode forward primer 10 and/or the hairpin barcode reverse primer 20. The adapter sequence 11, 21 and the stem sequence 15, 25 are configured to hybridize to each other at or under the closed annealing temperature and not hybridize to each other at or above the open annealing temperature.

In a particular embodiment, the hairpin barcode forward primer(s) 10 comprises, from the 5′ end 10A to the 3′ end 10B, the adapter sequence 11 comprising a 5′ stem sequence 12, the UMI sequence 17 comprising the structured UMI 14 and a 3′ stem sequence 15, and the target-specific sequence 16. In a particular embodiment, the barcode reverse primer(s) 20 comprises, from the 5′ end 20A to the 3′ end 20B, the adapter sequence 21 comprising a 5′ stem sequence 22, the UMI sequence 27 comprising the structured UMI 24 and a 3′ stem sequence 25, and the target-specific sequence 26. In these embodiments, at least a portion of the 5′ stem sequence 12, 22 of the hairpin barcode forward primer 10 and/or the hairpin barcode reverse primer 20 is complementary to at least a portion of the 3′ stem sequence 15, 25 of the hairpin barcode forward primer 10 and/or the hairpin barcode reverse primer 20. The 5′ stem sequence 12, 22 and the 3′ stem sequence 15, 25 are configured to hybridize to each other at or under the closed annealing temperature and not hybridize to each other at or above the open annealing temperature.

FIGS. 3A to 3C illustrate an embodiment where the hairpin barcode forward/reverse primer(s) 10, 20 comprises an adapter sequence 11, 21 comprising the 5′ stem sequence 12, 22 and a non-complementary sequence 13, 23 and the UMI sequence 17, 27 comprises the structured UMI 14, 24 and the 3′ stem sequence 15, 25. The 5′ and 3′ stem sequences 12, 22,15, 25 are then configured to hybridize to each other at or under the closed annealing temperature to form a loop comprising the non-complementary sequence 13, 23 and the structured UMI 14, 24. The structured UMI 14, 24 is thereby protected inside the loop of the hairpin barcode forward/reverse primer(s) 10, 20.

FIGS. 4A to 4C illustrate other embodiments of a hairpin barcode forward primer 10. These embodiments also apply to a corresponding hairpin barcode reverse primer 20. In FIG. 4A, the adapter sequence 11 does not comprise any non-complementary sequence but merely the so-called 5′ stem sequence 12. This means that when this 5′ stem sequence 12 is hybridized to the 3′ stem sequence 15 of the UMI sequence 17 at or under the closed annealing temperature the formed loop mainly consists of the structured UMI 14.

FIG. 4B illustrates another embodiment of a hairpin barcode forward primer 10. In this embodiment, the hybridizing portion 15 of the UMI sequence 17 is overlapping with the structured UMI 14. This means that at least a portion of the structured UMI 14 is complementary to at least portion 12 of the adapter sequence 11 to thereby hybridize to each other as shown in FIG. 4B at or under the closed annealing temperature. This is possible since the UMI 14 is a structured UMI 14 comprising predefined nucleotide sequences. The adapter sequence 11 can then be generated to have complementary nucleotides at corresponding positions to base pair with the predefined nucleotide sequences of the structured UMI 14. It is also possible to generate a nucleotide sequence in the adapter sequence 11 that is fully complementary to the nucleotides in the structured UMI 14 enzymatically, such as using a polymerase with the structured UMI 14 as template. In such a case, this portion 12 of the adapter sequence 11 can then be made complementary to the complete structured UMI 14 including both the random and predefined nucleotides of the structured UMI 14.

FIG. 4B also illustrates that the hairpin barcode forward primer 10 does not necessary have to form any loop. In clear contrast, the adapter sequence 11 and the UMI sequence 17 could hybridize to each other to protect the structured UMI 14 inside the hybridized part of the hairpin barcode forward primer 10.

FIG. 4C illustrates yet another embodiment of the hairpin barcode forward primer 10. In this embodiment, the adapter sequence 11 comprises the 5′ stem sequence 15 and the non-complementary sequence 13. The UMI sequence 17 comprises the structured UMI 14. In this embodiment, at least a portion of the structured UMI 14 is complementary to at least a portion of the 5′ stem sequence 12 so that the structured UMI 14 is hybridized to the 5′ stem sequence 12 at or under the closed annealing temperature. In this embodiment, the loop mainly consists of the non-complementary sequence 13 of the adapter sequence 11.

In an embodiment, see FIGS. 3A to 3C, 4A the UMI sequence 17, 27 comprises at least one destabilizing nucleotide 18, 28, preferably at least two destabilizing nucleotides 18, 28, between the structured UMI 14, 24 and the 3′ stem sequence 15, 25. Hence, in an embodiment, the hairpin barcode forward primer(s) 10 and/or the hairpin barcode reverse primer(s) 20 further comprises at least one destabilizing nucleotide 18, 28 between the structured UMI 14, 24 and the 3′ stem sequence 15, 25. In a particular embodiment, the hairpin barcode forward primer(s) 10 and/or the hairpin barcode reverse primer(s) 20 comprise at least two destabilizing nucleotides 18, 28, between the structured UMI 14, 24 and the 3′ stem sequence 15, 25. Such destabilizing nucleotide(s) can be incorporated to ensure that the structured UMI 14, 24 itself does not, by random chance, complement the adapter sequence 11, 21 and results in a longer, more stable stem having higher closed and open annealing temperatures.

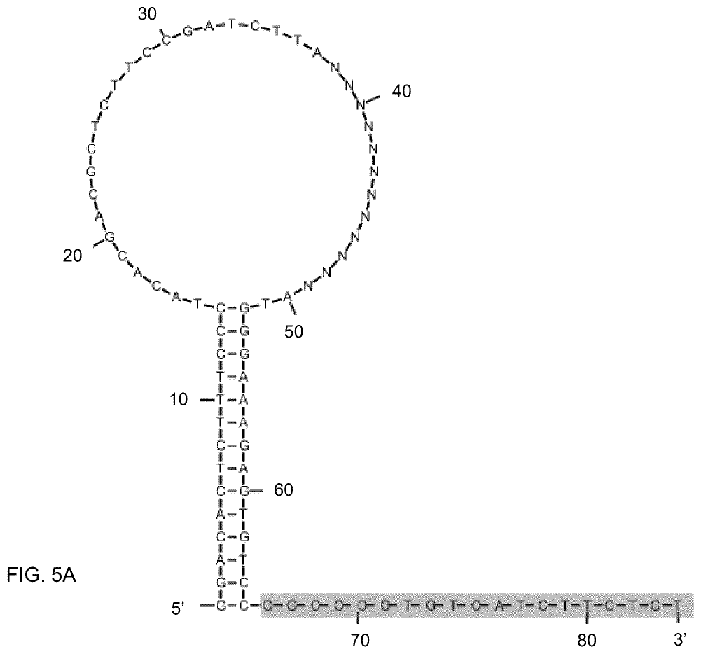

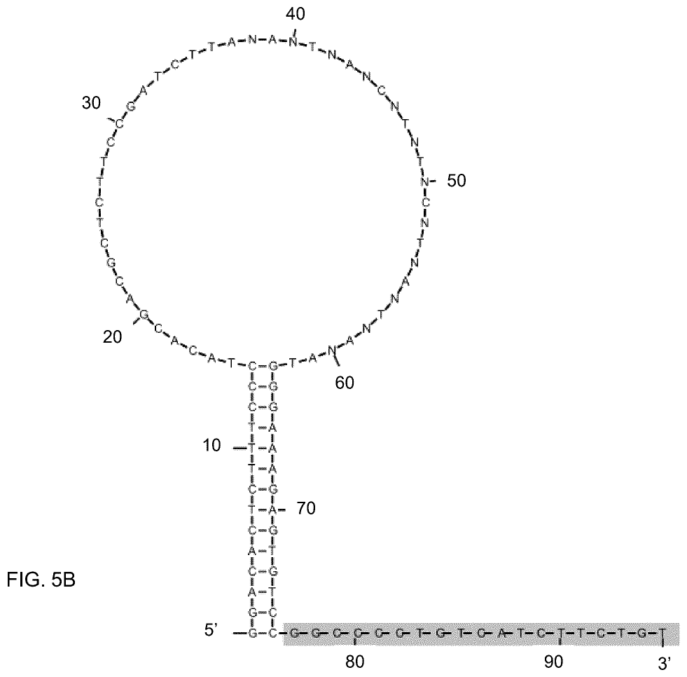

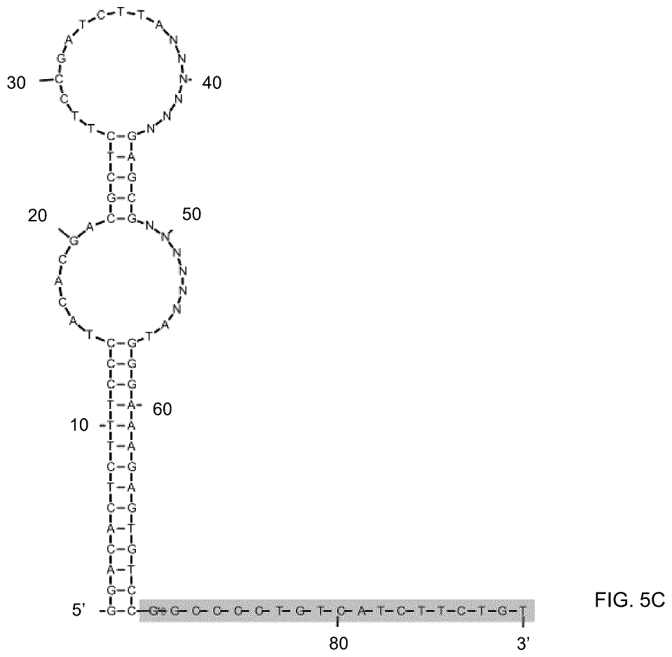

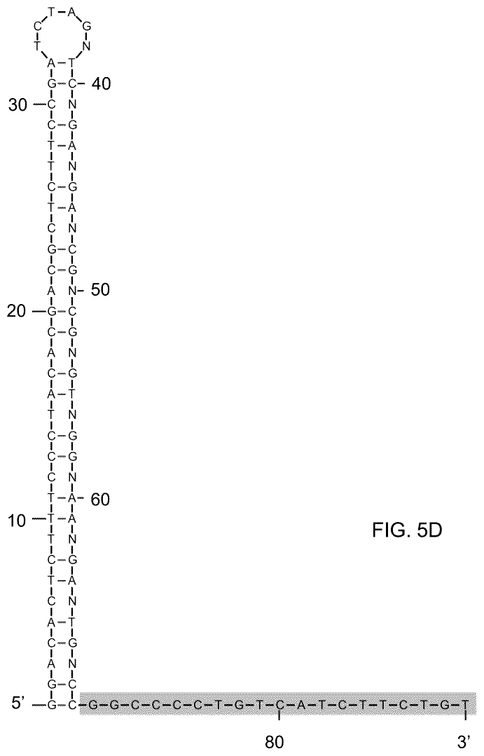

FIG. 5A illustrates a standard hairpin barcode primer with a continuous UMI, i.e., not a structured UMI. FIG. 5B illustrates an embodiment of a forward hairpin barcode primer with a structured UMI, in which the random nucleotides (N) are distributed over the structured UMI with alternating predefined nucleotides and alternating random nucleotides. FIG. 5C illustrates an embodiment of a double-loop forward hairpin barcode primer having a double loop with structured UMI with a first UMI part in a first loop and a second UMI part in a second loop and with predefined nucleotides between the first and second UMI parts. These predefined nucleotides are capable of hybridizing to complementary nucleotides of the adapter sequence. FIG. 5D illustrates an embodiment of a forward barcode primer with an integrated barcode partly forming part of the loop and partly forming part of the hybridizing portion of the UMI sequence.

The structured UMI 14, 24 of the hairpin barcode forward primers 10 or of the hairpin barcode reverse primers 20 could include the same number of nucleotides. In such an embodiment, all the structured UMIs 14, 24 have the same length in terms of number of nucleotides. The embodiments are, however, not limited thereto. In other embodiments, a further level of “randomization” of the structured UMIs 14, 24 could be achieved by having different, such as random, number of nucleotides in the structured UMIs 14, 24 in different hairpin barcode forward or reverse primers 10, 20. In the latter case, this could be achieved by (randomly) providing a length of a structured UMI 14, 24 within a predefined interval of available lengths, such as from u nucleotides up to v nucleotides, wherein u, v are positive numbers and u<v. In such an embodiment, the different hairpin barcode forward primers 10 and/or hairpin barcode reverse primers 20 have lengths of the structured UMIs 14, 24 within the interval of from u up to v but not all such hairpin barcode primers 10, 20 need to have the same lengths of their respective structured UMIs 14, 24.

In the following various embodiments of the structured UMI 14, 24 are described. In these embodiments, reference is typically made to nucleotides in terms of A, T, C or G. In such embodiments, the hairpin barcode forward/reverse primer(s) 10, 20 are DNA primers. These embodiments, however, also apply to RNA primers, in which case the nucleotides are A, U, C or G. It is also possible to include modified nucleotides and nucleic acid analogues, for instance, PNA, LNA, GNA, TNA and/or ZNA, in the structured UMI 14, 24.

In an embodiment, the structured UMI 14, 24 comprises two UMI parts 14A, 14B, 24A, 24B separated by one predefined nucleotide sequence 14C, 24C. In such a case, the structured UMI 14, 24 could be in the form of N₁N₂ . . . Nₖ_{₁}M₁ . . . MₗNₖ_{₁}₊₁Nₖ_{₁}₊₂ . . . Nₖ_{₂}, wherein N₁N₂ . . . Nₖ_{₁} represents the first UMI part 14A, 24A, Nₖ_{₁}₊₁Nₖ_{₁}₊₂ . . . Nₖ_{₂} represents the second UMI part 14B, 24B and M₁ . . . Mₗ represents the predefined nucleotide sequence 14C, 24C. In such an embodiment, each N is independently a random nucleotide among A, T, C and G, each M is independently a respective predefined nucleotide among A, T, C and G, and k₁, k₂ and l are positive integers.

In a preferred embodiment, the structured UMI 14, 24 comprises more than two UMI parts 14A, 14B, 24A, 24B separated by a respective predefined nucleotide sequence 14C, 24C. In such an embodiment, the structured UMI 14, 24 comprises alternating UMI parts 14A, 14B, 24A, 24B and predefined nucleotide sequences 14C, 24C.

In an embodiment, each UMI part 14A, 14B, 24A, 24B of the structured UMI 14, 24 comprises a same number of nucleotides. This same number of nucleotides is preferably selected within a range of from one up to eight, preferably from one up to six, and even more preferably from one up to four, such as one, two, three or four.

In another embodiment, at least two UMI parts 14A, 14B, 24A, 24B of the structured UMI 14, 24 comprise or have different number of nucleotides. Hence, in this embodiment, not all UMI parts 14A, 14B, 24A, 24B of a structured UMI 14, 24 have the same length in terms of number of nucleotides. The length of the different UMI parts 14A, 14B, 24A, 24B may be selected within a range of from one up to eight, preferably from one up to six, and even more preferably from one up to four, such as one, two, three or four.

In an embodiment, if the structured UMI 14, 24 comprises multiple predefined nucleotide sequences 14C, 24C they may all have the same number of nucleotides. In another embodiment, at least two predefined nucleotide sequences 14C, 24C of a structured UMI 14, 24 comprise different number of nucleotides and thereby have different lengths.

In an embodiment, the structured UMI 14, 24 comprises multiple UMI parts 14A, 14B, 24A, 24B separated by a respective predefined nucleotide sequence 14C, 24C.

The number of nucleotides in the predefined nucleotide sequence(s) 14C, 24C of a structured UMI 14, 24 could have the same or different number of nucleotides as compared to the number of nucleotides in the UMI parts 14A, 14B, 24A, 24B of the structured UMI 14, 24. In a preferred embodiment, the predefined nucleotide sequence(s) 14C, 24C of a structured UMI 14, 24 has the same or lower number of nucleotides as compared to the UMI parts 14A, 14B, 24A, 24B of the structured UMI 14, 24.

As mentioned in the foregoing, a preferred embodiment of structured UMI 14, 24 comprises alternating UMI parts 14A, 14B, 24A, 24B, each comprising one or more random nucleotides, and respective predefined nucleotide sequences 14C, 24C, each comprising one or more predefined nucleotides.

In an embodiment, the structured UMI 14, 24 is selected from the group consisting of

$$\begin{matrix}{N}_{1} {M}_{1} {N}_{2} {M}_{2} {N}_{3} {M}_{3} … {N}_{k} ; \\ {N}_{1} {M}_{1} {N}_{2} {M}_{2} {N}_{3} {M}_{3} … {N}_{k} {M}_{k} ; \\ {N}_{1} … {N}_{{k}_{1}} {M}_{1} {N}_{{k}_{1} + 1} … {N}_{{k}_{2}} {M}_{2} {N}_{{k}_{2} + 1} … {N}_{{k}_{n}} ; \\ {N}_{1} … {N}_{{k}_{1}} {M}_{1} {N}_{{k}_{1} + 1} … {N}_{{k}_{2}} {M}_{2} {N}_{{k}_{2} + 1} … {N}_{{k}_{n}} {M}_{n} ; \\ {N}_{1} … {N}_{{k}_{1}} {M}_{1} … {M}_{{l}_{1}} {N}_{{k}_{1} + 1} … {N}_{{k}_{2}} {M}_{{l}_{1} + 1} … {M}_{{l}_{2}} … {N}_{{k}_{n - 1} + 1} … {N}_{{k}_{n}} ; and \\ {N}_{1} … {N}_{{k}_{1}} {M}_{1} … {M}_{{l}_{1}} {N}_{{k}_{1} + 1} … {N}_{{k}_{2}} {M}_{{l}_{1} + 1} … {M}_{{l}_{2}} … {N}_{{k}_{n - 1} + 1} … {N}_{{k}_{n}} {M}_{{l}_{n - 1} + 1} … {M}_{{l}_{n}} .\end{matrix}$$

In this embodiment, each N is independently a random nucleotide among A, T, C and G (or A, U, C and G) and each Mis independently a respective predefined nucleotide among A, T, C and G (or A, U, C and G). k is an integer from 3 up to 20, preferably from 4 up to 20, and more preferably from 6 up to 15, each k, i=1 . . . n is independently an integer from 2 up to 8, preferably from 2 up to 6, and more preferably from 2 up to 4 and each lⱼ, j=1 . . . n is independently an integer from 1 up to 8, preferably from 1 up to 6, and more preferably from 1 up to 4. N is an integer from 3 up to 20, preferably from 4 up to 20, and more preferably from 6 up to 15. It is also possible to use longer structured UMIs 14, 24, such as by having k larger than 20.

It is also possible to provide structured UMI 14, 24 where the random nucleotides are randomly among a subset of the available nucleotides A, T, C and G (or A, U, C and G). For instance, the random nucleotide could be a random nucleotide among three or these available nucleotides A, T, C and G (or A, U, C and G).

In a particular embodiment, each lⱼ is equal to or lower than each k.

In an embodiment, the UMI 14, 24 is N₁ . . . Nₖ_{₁}M₁ . . . Mₗ_{₁}Nₖ_{₁}₊₁ . . . Nₖ_{₂}MNₖ_{₂}₊₁ . . . Nₖ_{₃}Mₗ_{₁}₊₁ . . . Mₗ_{₂}Nₖ_{₃}₊₁ . . . Nₖ_{₄}.

In a particular embodiment, each Nis independently a random nucleotide among A, T, C and G and each M is a respective predefined nucleotide among A, T, C and G, preferably A. Hence, in this particular embodiment, all predefined nucleotides are the same nucleotide, i.e., all are A, all are T, all are C or all are G, preferably all are A.

In this particular embodiment, the structured UMI 14, 24 consists of four UMI parts separated by a respective sequence of at least one predefined nucleotide. In an embodiment, the first and fourth UMI parts (first and last UMI parts) have the same number of nucleotides, i.e., k₁−1=k₄−k₃₊₁. In an embodiment, the second and third UMI parts (intermediate UMI parts) also have the same number of nucleotides, i.e., k₂−k₁₊₁=k₃−k₂₊₁. Furthermore, the first and third sequences of at least one predefined nucleotide preferably have the same number of predefined nucleotides, i.e., l₁−1=l₂−l₁₊₁.

In a particular preferred embodiment, the structured UMI 14, 24 is N₁N₂N₃N₄M₁M₂M₃N₅N₆M₄N₇N₈M₅M₆M₇N₉N₁₀N₁₁N₁₂.

In the above-described embodiment, the UMI parts 14A, 14B, 24A, 24B of the structured UMI 14, 24 are exemplified as comparing fully random nucleotides, i.e., N. It is, however, possible that at least one or more of the nucleotides of the UMI parts 14A, 14B, 24A, 24B of the structured UMI 14, 24 is or are degenerated nucleotides, e.g., W, S, M, K, R, Y, B, D, H or V.

In the embodiments described above, the respective M could a predefined degenerative nucleotide such as W or S. For instance, a predefined nucleotide W for M implies that Mis A or T (or A or U) but is not G or C. Correspondingly, a predefined nucleotide S for M means that Mis G or C but not A or T (or A or U).

Particular examples of structured UMIs 14, 24 that could be used in the hairpin barcode primer 10, 20 include NNNNMMMNNMNNMMMNNNN, NNNMMNNNMMNNNMMNNN, and NNNMMNNMMNNMMNNN, in which each Mis the same predefined nucleotide selected among A, T, C and G, such as A. Alternative versions of these structured UMIs 14, 24 include NNNNSWSNNWNNSWSNNNN, NNNNWSWNNSNNWSWNNNN, NNNSWNNNSWNNNSWNNN, NNNWSNNNWSNNNWSNNN, NNNSWNNSWNNSWNNN and NNNWSNNWSNNWSNNN. This alternative versions have alternating S and W as predefined nucleotide M, which gives a balanced GC content of the structured UMIs 14, 24.

Each hairpin barcode primer 10 and/or hairpin barcode reverse primer 20 preferably comprises a respective unique structured UMI 14, 24 having a random sequence that is different from the random sequences of structured UMIs 14, 24 in other hairpin barcode primers 10 and/or hairpin barcode reverse primers 20. The total length of the structured UMIs 14, 24 is equal to the combined lengths of the UMI parts 14A, 14B, 24A, 24B. This total length of the structured UMIs 14, 24 is preferably selected at least partly based on the number of nucleic acid molecules 1 in the sample. For instance, the number of unique structured UMIs 14, 24 is equal for 4^{h} for a structured UMI 14, 24 having a total length of h nucleotides. This number 4^{h} should preferably be significantly larger than the number of nucleic acid molecules 1 in the sample.

The amplification of the nucleic acid molecule in step S2 in FIG. 1 and step S11 in FIG. 2 comprises performing PCR pre-amplification at an annealing temperature equal to or less than the closed annealing temperature of the hairpin barcode forward primer(s) 10 and/or the hairpin barcode reverse primer(s) 20. Accordingly, at this PCR pre-amplification in step S2 and S11 at least a majority of the hairpin barcode forward primers 10 and/or hairpin barcode reverse primers 20 have an intact hairpin loop, i.e., the adapter sequence 11, 21 is hybridized to the UMI sequence 17, 27. This in turn significantly reduces the amount of non-specific PCR products that may otherwise occur during the pre-amplification by the random nucleotide sequence of the structured UMI 14, 24. Hence, the vast majority of the PCR products from the PCR-amplification in step S2 and S11 are the desired barcoded PCR products 50 corresponding to an amplified portion 2, 3, 4 of the (target) nucleic acid molecule 1.

The closed annealing temperature Is lower than a melting temperature of the adapter sequence 11, 21 and the UMI sequence 17, 27. Correspondingly, the open annealing temperature is higher than a melting temperature of the adapter sequence 11, 21 and the UMI sequence 17, 27.

In an example, the closed annealing temperature is equal to or less than 65° C. Hence, in such an example, the PCR pre-amplification of step S2 and S11 is preferably performed at an annealing temperature equal to or less than to 65° C. For instance, the PCR pre-amplification of step S2 and S11 could be performed at an annealing temperature selected within an interval of from 60° C. up to 65° C., preferably from 60° C. up to 64° C., such as at about 62° C.

The PCR pre-amplification of the (target) nucleic acid molecule 1 is performed using a polymerase, preferably a DNA polymerase, and more preferably a heat-stable DNA polymerase. Non-limiting, but illustrative, examples of DNA polymerases that can be used according to the embodiments include Thermus thermophilus (Tth) DNA polymerase, Bacillus stearothermophilus DNA polymerase, Thermus aquaticus (Taq) DNA polymerase, Thermus flavus (Tfl) polymerase, Vent® DNA polymerase, Pfu polymerase, and Escherichia coliDNA polymerase I. In some embodiments, the DNA polymerase lacks 5′-nuclease activity. Examples of such polymerases include Klenow fragment of DNA polymerase 1, Stoeffel fragment of Taq polymerase, Pfu polymerase or Vent® polymerase. In an embodiment, the DNA polymerase is a so-called thermoactivated DNA polymerase, also referred to as, hot-start DNA polymerase. Specific examples of DNA polymerases include Takara PRIME STAR GXL polymerase I, Clontec's ADVANTAGE HD Polymerase, NEB Q5® High-Fidelity DNA Polymerases NEB PHUSION® High-Fidelity DNA Polymerases, ThermoFisher PLATINUM® Taq DNA Polymerase High Fidelity, ThermoFisher ACCUPRIME™ Pfx DNA Polymerase, ThermoFisher ACCUPRIME™ Taq DNA Polymerase High Fidelity, ThermoFisher Phusion™ High Fidelity Polymerase, ThermoFisher Platinum™ SuperFi™ II DNA Polhymerase, Promega Pfu DNA Polymerase, and Qiagen HOTSTAR HIFIDELITY Polymerase. The above presented examples of DNA polymerases that can be used in the PCR pre-amplification in step S2 and S11 may also be used in the PCR amplification in step S4 in FIG. 1 and step S13 in FIG. 2.

In an embodiment, step S2 and S11 comprises amplifying the (target) nucleic acid molecules 1 by performing 1-40 cycles of PCR pre-amplification of the (target) nucleic acid molecules 1 to form the plurality of barcoded PCR products 50. In preferred embodiments, step S2 and S11 comprises amplifying the (target) nucleic acid molecules 1 by performing 1-30 cycles, and more preferably 1-25 cycles, such as 2-20 cycles and more preferably 2-15 cycles of PCR pre-amplification of the (target) nucleic acid molecules 1 to form the plurality of barcoded PCR products 50. Hence, it is generally preferred to perform a rather low number of PCR cycles in the PCR pre-amplification in step S2 and S11. This low number of PCR cycles, together with the protecting structured UMIs 14, 24 with Q hairpin loops, significantly reduces the amount of non-specific PCR products produced in step S2 and S11.

The barcoded PCR products 50 obtained in the amplification in step S2 and S11 are then contacted in step S3 or S12 with an adapter-specific forward primer 30 and an adapter-specific reverse primer 40 as indicated in FIG. 3A.

In an embodiment, the adapter-specific forward primer 30 comprises a sequence equal to or complementary to, preferably equal to, the adapter sequence 13 of the forward primer(s) 10, such as of the hairpin barcode forward primer(s) 10. Correspondingly, the adapter-specific reverse primer 40 comprises a sequence equal to or complementary to, preferably equal to, the adapter sequence 23 of the reverse primer(s) 20.

In a particular embodiment, the adapter-specific forward primer 30 comprises, from a 5′ end 30A to a 3′ end 30B, one of a P5 sequence and a P7 sequence 32 and the sequence 33 equal to or complementary to, preferably equal to, at least a portion of the adapter sequence 11 of the forward primer(s) 10. In this particular embodiment, the adapter-specific reverse primer 40 comprises, from a 5′ end 40A to a 3′ end 40B, the other of the P5 sequence and the P7 sequence 42 and the sequence 43 equal to or complementary to, preferably equal to, at least a portion of the adapter sequence 21 of the reverse primer(s) 20. In a particular embodiment, the adapter-specific reverse primer 40 comprises, from the 5′ end 40A to the 3′ end 40B, the other of the P5 sequence and the P7 sequence 42, an index sequence and the sequence 43 equal to or complementary to, preferably equal to, at least a portion of the adapter sequence 21 of the reverse primer(s) 20.

In an embodiment, the P5 and P7 sequences are P5 and P7 ILLUMINA® sequences. In an embodiment, the P5 sequence comprises AATGATACGGCGACCACCGA (SEQ ID NO: 6). For instance, the P5 sequence could comprise, such as consist of, AATGATACGGCGACCACCGAGATCTACAC (SEQ ID NO: 7). In this latter example, at least one of the 3′ nucleotides of the P5 sequence may be common for the P5 sequence and the following sequence 33. Correspondingly, in an embodiment, the P7 comprises, preferably consists of, CAAGCAGAAGACGGCATACGAGAT (SEQ ID NO: 8).

The PCR amplification in step S4 and S13 is performed at an annealing temperature equal to or greater than the open annealing temperature of the hairpin barcode forward primer(s) 10 and/or the hairpin barcode reverse primer(s) 20. At such an annealing temperature, at least a significant portion of the barcoded PCR products 50 are in an open structure, i.e., there is no significant hairpin loop formation by hybridization of complementary stem portions of the barcoded PCR products 50. In an example, the open annealing temperature is at least 70° C. In such a particular example, the PCR amplification in step S4 is performed at a temperature equal to or above 70° C., such as 71° C., 72° C. or even higher.

In an embodiment, the open annealing temperature of the hairpin barcode forward primer(s) 10 and/or the hairpin barcode reverse primer(s) 20 is higher than the closed annealing temperature of the hairpin barcode forward primer(s) 10 and/or the hairpin barcode reverse primer(s) 20. In a particular embodiment, the open annealing temperature is at least 1° C., preferably at least 2° C., such at least 3° C. or 4° C., and more preferably at least 5° C. higher than the closed annealing temperature.

In an embodiment, step S4 in FIG. 1 and step S13 in FIG. 2 comprise amplifying the barcoded PCR products 50 by performing at least 2 cycles of PCR amplification on the barcoded PCR products 50 to form (a library of) amplified barcoded PCR products 60. In preferred embodiments, step S4 and S13 comprise amplifying the barcoded PCR products 50 by performing at least 3 cycles, such as at least 5 cycles, and more preferably at least 10 cycles of PCR amplification on the barcoded PCR products 50 to form the (library of) amplified barcoded PCR products 60. For instance, at least 15 cycles, such as at least 20 cycles, such as at least 25 cycles or at least 30 cycles of PCR amplification are performed on the barcoded PCR products 50 to form the (library of) amplified barcoded PCR products 60. The number of PCR cycles is preferably selected to achieve sufficient number of amplified barcoded PCR products 60 for sequencing even for nucleic acid molecules 1 present in a low copy number in the sample.

In a general embodiment, the number of PCR cycles in the amplification in step S4 and S13 is higher than the number of PCR cycles in the amplification in step S2 and S11.

In an optional, but preferred embodiment, the method comprises an additional step following step S2 and prior to step S4 in FIG. 1 and following step S11 and prior to step S13 in FIG. 2. This additional step then comprises degrading the polymerase used for amplifying the (target) nucleic acid molecules 1 in the PCR pre-amplification (in step S2 or S11) prior to amplifying the barcoded PCR products in the PCR amplification (in step S4 or S13).

Various techniques could be used to degrade polymerases including, but not limited to, heat treatment, chemical treatment, addition of protease inhibitors, sample dilution and enzymatic treatment. For instance, a protease could be added to the amplification products from step S2 or S11 to enzymatically degrade the polymerase used for amplifying the (target) nucleic acid molecules 1 in the PCR pre-amplification. Such a degradation of the polymerase once the PCR pre-amplification has been completed additionally inhibits formation of non-specific PCR products.

With reference to FIG. 2, in an optional embodiment, the library of amplified barcoded PCR products 60 obtained in step S13 is first cleaned up prior to sequencing in step S14. For instance, the library may be purified to remove contaminants, such as dNTPs, salts, primers, etc., that may otherwise interfere with the sequencing. An example of such a purification is to use beads, such as magnetic beads, to bind the amplified barcoded PCR products 60 and thereby remove the amplified barcoded PCR products 60 from the remaining reagents from the PCR amplification in step S13. An illustrative, but non-limiting, example of a purification kit that could be used is AMPure XP for PCR Purification.

The amplified barcoded PCR products 60 are then sequenced in step S14 of FIG. 2. The sequencing is achieved by means of at least one sequencing primer. The result of the sequencing in step S14 is respective sequence reads comprising at least nucleotide sequences of the UMI 64 and nucleic acid molecule sequence(s) 66.

In a particular embodiment, step S14 comprises in situ sequencing the at least a portion of the amplified barcoded PCR products 60 immobilized onto a solid support. For instance, the preferred P5 and P7 sequences introduced into the amplified barcoded PCR products 60 by means of the adapter-specific forward primers 30 and the adapter-specific reverse primers 40 could be used to immobilize the amplified barcoded PCR products 60 onto the solid support. In such an embodiment, the solid support preferably comprises immobilized nucleotide sequences complementary to the P5 sequence and/or immobilized nucleotide sequences complementary to the P7 sequence.

The in situ sequencing of step 56 preferably comprises in situ sequencing by synthesis of the at least a portion of the amplified product 60.

For instance, if the adapter-specific forward and reverse primers 30, 40 comprise P5 and P7 sequences, respectively, the ILLUMINA® sequencing technology could be used to in situ sequence at least a portion of the amplified barcoded PCR products 60 by synthesis. In more detail, the amplified barcoded PCR products 60 are immobilized on a flow cell surface designed to present the amplified barcoded PCR sequences 60 in a manner that facilitates access to enzymes while ensuring high stability of surface bound amplified barcoded PCR products 60 and low non-specific binding of fluorescently labeled nucleotides.

Sequence By Synthesis (SBS) uses two or four fluorescently labeled nucleotides to sequence the amplified barcoded PCR products 60 on the flow cell surface in parallel. During each sequencing cycle, a single labeled deoxynucleoside triphosphate (dNTP) is added to the nucleic acid chain. The nucleotide label serves as a terminator for polymerization so after each dNTP incorporation, the fluorescent dye is imaged to identify the base and then enzymatically cleaved to allow incorporation of the next nucleotide.

As an illustrative, but non-limiting, example ILLUMINA® MiniSeq system could be used to sequence the amplified barcoded PCR products 60.

Accurate quantification and sequence error correction with UMIs is predicated on a one-to-one relationship between the number of unique UMI barcodes at a given genomic locus and the number of unique sequence reads that have been sequenced. However, errors within the UMI sequence, including nucleotide substitutions during amplification and nucleotide miscalling and insertions or deletions (indels) during sequencing, create additional artefactual UMIs.

There are different bioinformatics models and methods for determining whether UMIs have the same nucleotide sequence or are regarded as being different UMIs, e.g., UMI error resolving methods (Genome Research (2017) 27(3): 491-499). The simplest method is to merge all UMIs, retaining only the UMI with the highest counts. For this method, the number of networks formed at a given locus is equivalent to the estimated number of unique molecules. This method is often referred to as cluster method. This method is expected to underestimate the number of unique molecules, especially for complex networks. A more accurate method is denoted adjacency method (Genome Research (2017) 27(3): 491-499), which attempts to correctly resolve the complex networks by using node counts. The most abundant node and all nodes connected to it are removed from the network. If this does not account for all the nodes in the network, the next most abundant node and its neighbors are also removed. This is repeated until all nodes in the network are accounted for. In the method, the total number of steps to resolve the network(s) formed at a given locus is equivalent to the number of estimated unique molecules. This method allows a complex network to originate from more than one UMI, although UMIs with an edit distance of two will always be removed in separate steps. The excess of UMIs pairs with an edit distance of two observed in the data sets indicate that some of these UMIs are artefactual. Reasoning that counts for UMIs generated by a single sequencing error should be higher than those generated by two errors and UMIs resulting from errors during the PCR amplification stage should have higher counts than UMIs resulting from sequencing errors, the directional method was developed (Genome Research (2017) 27(3): 491-499). Networks were generated from the UMIs at a single locus, in which directional edges connect nodes a single edit distance apart when nₐ≥2n_{b}−1, where nₐ and n_{b} are the counts of node a and node b. The entire directional network is then considered to have originated from the node with the highest counts. The ratio between the final counts for the true UMI and the erroneous UMI generated from a PCR error is dependent upon which PCR cycle the error occurs and the relative amplification biases for the two UMIs, but should rarely be less than twofold. This method allows UMIs separated by edit distances greater than one to be merged so long as the intermediate UMI is also observed, and with each sequential base change from the most abundant UMI, the count decreases. For this method, the number of directional networks formed is equivalent to the estimated number of unique molecules.

The sequence reads as obtained in step S14 are then demultiplexed based on the nucleic acid sequences of the UMIs 64 in step S15. In an embodiment, this step S15 comprises dividing the sequence reads into groups having a same nucleotide sequence of the UMIs 64, optionally with at most a predefined number of mismatches allowed for nucleotide sequences of UMIs 64 in a same group. In a particular embodiment, this predefined number is zero, one or two, and preferably zero or one.

Thus, the sequence reads are preferably divided into different groups based on the nucleotide sequences of the UMIs so that sequence reads having the same UMI sequence are included in the same group. In this grouping or division of sequence reads, it is possible, in an embodiment, to accept up to two nucleotide mismatches, preferably up to one mismatch, between UMI sequences that belong to the same group.

The demultiplexed sequence reads are then mapped in step S16 to respective nucleic acid molecules based on nucleic acid sequences of the nucleic acid molecule sequences 66 in the sequence reads. In an embodiment, this step S16 comprises dividing demultiplexed sequence reads into groups having a same nucleotide sequence of the nucleic acid molecule sequences 66, optionally with at most a predefined number of mismatches allowed for nucleotide sequences of nucleic acid molecule sequences 66 in a same group. In a particular embodiment, the predefined number is an integer equal to or larger than 0 but no larger than ten, preferably no larger than nine, such as no larger than eight or seven, or even no larger than six or five. Thus, within each group of sequence reads following the demultiplexing in step S15, the sequence reads are further divided into groups, or sub-groups, based on the nucleotide sequences of the nucleic acid molecule sequences 66. This means that sequence reads having the same nucleic acid molecule sequences 66 are grouped into the same group. In this grouping or division of sequence reads, it is possible, in an embodiment, to accept up to 10 nucleotide mismatches, preferably up to 5-6 nucleotide mismatches, between nucleic acid molecule sequences 66 that belong to the same group. Mismatches are allowed in order to accept possible polymerase-caused nucleotide substitutions during library preparation and sequencing procedure.

The amplification method of the invention may simultaneously, i.e., in the same reaction vessel, amplify multiple different target nucleic acid sequences. In such a case, a sample comprising nucleic acid molecules 1 is contacted with P forward primers 10 and Q reverse primers 20 in step S1 of FIG. 1.

In an embodiment, P is an integer equal to or larger than one and Q is an integer equal to or larger than one. The P forward primers 10 comprise, from a 5′ end 10A to a 3′ end 10B, an adapter sequence 11 and a target-specific sequence 16. Correspondingly, the Q reverse primers 20 comprise, from a 5′ end 20A to a 3′ end 20B, an adapter sequence 21 and a target-specific sequence 26.

In an embodiment, the P forward primers 10 are P hairpin barcode forward primers 10 and/or the Q reverse primers 20 are Q hairpin barcode reverse primers 20. In such an embodiment, each hairpin barcode forward primer 10 comprises, from the 5′ end 10A to the 3′ end 10B, the adapter sequence 11, a UMI sequence 17 comprising a structured UMI 14, and the target-specific sequence 16 complementary to a respective portion 2 of a nucleic acid molecule 1. Correspondingly, each hairpin barcode reverse primer 20 comprises, from the 5′ end 20A to the 3′ end 20B the adapter sequence 21, a UMI sequence comprising a structured UMI 24, and the target-specific sequence 26 complementary to a respective portion 3 of a nucleic acid molecule 1. At least a portion 12, 22 of the adapter sequence 11, 21 of the hairpin barcode forward primer 10 and/or the hairpin barcode reverse primer 20 is complementary to at least a portion 15, 25 of the UMI sequence 17, 27 of the hairpin barcode forward primer 10 and/or the hairpin barcode reverse primer 20. The adapter sequence 11, 21 and the UMI sequence 17, 27 are configured to hybridize to each other at or under a closed annealing temperature and not hybridize to each other at or above an open annealing temperature.

This embodiment of the method also comprises amplifying, in step S2, the nucleic acid molecules 1 by performing PCR pre-amplification of the nucleic acid molecules 1 to form a plurality of barcoded PCR products 50. The PCR pre-amplification as performed in step S2 has an annealing temperature equal to or less than the closed annealing temperature of the hairpin barcode forward primers 10 and/or the hairpin barcode reverse primers 20.

The embodiment further comprises contacting, in step S3, the plurality of barcoded PCR products 50 with an adapter-specific forward primer 30 and an adapter-specific reverse primer 40 and amplifying, in step S4, the barcoded PCR products 50 by performing PCR amplification on the barcoded PCR products 50 to form a library of amplified barcoded PCR products 60. In an embodiment, at least a portion of cycles of the PCR amplification has an annealing temperature greater than or equal to the open annealing temperature of the hairpin barcode forward primers 10 and/or the hairpin barcode reverse primer 20.

In an embodiment, the amplification method also comprises sequencing at least a respective portion of the amplified barcoded PCR products 60 to form respective sequence reads comprising the UMI(s) 64 and nucleic acid sequence(s) 66. The method further comprises demultiplexing the sequence reads based on nucleic acid sequences of the UMIs 64 and mapping the demultiplexed sequence reads to respective target nucleic acid sequences based on the sequences of the nucleic acid sequences 66.

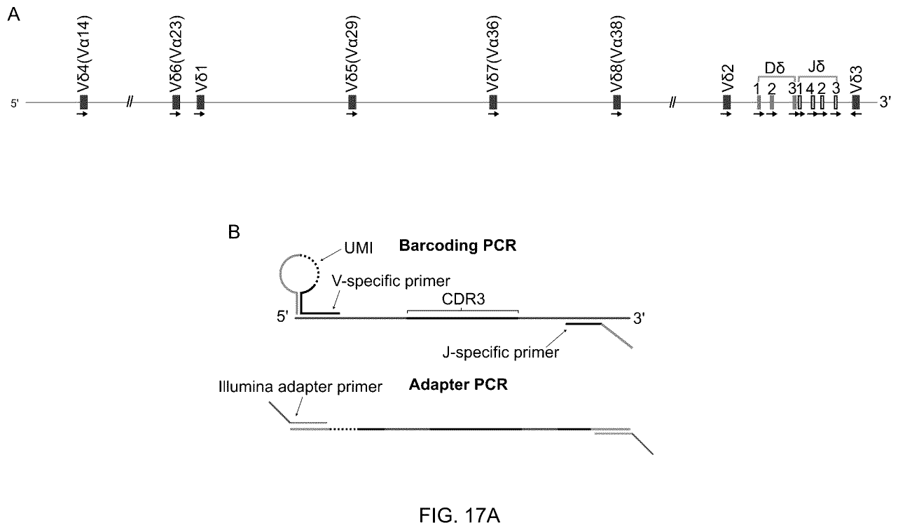

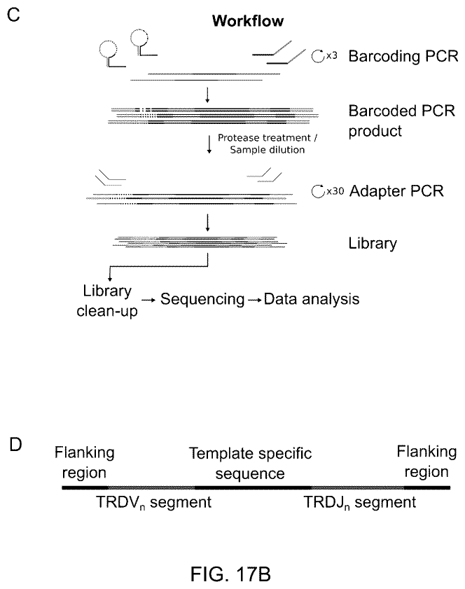

The method of quantifying nucleic acid molecules of the embodiments may advantageously be used to determine lymphocyte clonality, see FIGS. 17A-17D.

The sequencing of immunoreceptor repertoires of B and T lymphocytes and tracking of specific clones provide essential information regarding many aspects of immunity, such as the extent of immune responses, localization of specific responding cells, the duration of immune responses, and presence of immune memory. An embodiment of the invention provides an ultrasensitive immune repertoire sequencing approach that can be used to determine lymphocyte clonality, such as represented by T-cell receptor (TCR) and/or B-cell receptor (BCR) clonotypes in a sample.

There are several challenges when analyzing lymphocyte clonality in a sample including, for instance, determining the exact number and size of the TCR and/or BCR clonotypes, no prior information of the number of different TCR and/or BCR clonotypes in the sample, distinguishing between clonotypes with similar sequences from sequence errors introduced during amplification and/or sequencing.

As used herein, each T cell or B cell clone carries a unique clonotype defined by the nucleotide sequence that arises during the gene rearrangement process for the TCR or BCR. In most applications, the clonotype is regarded as being defined by the complementarity-determining region 3 (CDR3) of the TCR or BCR, such as the CDR3 of the alpha (α) chain of the TCR (TRA) for αβ TCRs and T cells, or more preferably the CDR3 of the beta (β) chain of the TCR (TRB) for αβ TCRs and T cells, or the CDR3 of the gamma (γ) chain of the TCR (TRG) for γδ TCRs and T cells, or more preferably the CDR3 of the delta (δ) chain of the TCR (TRD) for γδ TCRs and T cells, or the CDR3 of the BCR for B cells.

The TCR alpha (α) chain is generated by recombination of variable (V) and joining (J) gene segments, whereas the TCR beta (β) chain is generated by recombination the V, diversity (D) and joining (J) gene segments. Correspondingly, the TCR gamma (γ) chain involves V and J recombination, whereas the TCR delta (δ) chain is generated by V, D and J recombination.

Determination of lymphocyte clonality could thereby be used to determine the particular combination of V (TRAV) and J (TRAJ) gene segments of the TCR alpha (α) chain and/or the particular combination of V (TRBV) and D (TRBD) gene segments, D and J (TRBJ) gene segments or V, D and J gene segments of the TCR beta (β) chain for αβ T cells. Correspondingly, determination of lymphocyte clonality could be used to determine the particular combination of V (TRGV) and J (TRGJ) gene segments of the TCR gamma (γ) chain and/or the particular combination of V (TRDV) and D (TRDD) gene segments, D and J (TRDJ) gene segments or V, D and J gene segments of the TCR delta (δ) chain for γδ T cells.

The BCR is also generated based on V, D and J recombination. This means that determination of lymphocyte clonality could be used to determine the particular combination of V and J gene segments, D and J gene segments or V, D and J gene segments of the BCR for B cells.

In an embodiment, a method of determining lymphocyte clonality, see FIGS. 2, 3A to 3C and 4A to 4C, comprises contacting, in step S10, a sample comprising nucleic acid molecules 1 of lymphocytes with P forward primers 10 and Q reverse primers 20.

In an embodiment, P is an integer equal to or larger than one and Q is an integer equal to or larger than one. The P forward primers 10 comprise, from a 5′ end 10A to a 3′ end 10B, an adapter sequence 11 and a target-specific sequence 16. Correspondingly, the Q reverse primers 20 comprise, from a 5′ end 20A to a 3′ end 20B, an adapter sequence 21 and a target-specific sequence 26.

In an embodiment, the P forward primers 10 are P hairpin barcode forward primers 10 and/or the Q reverse primers 20 are Q hairpin barcode reverse primers 20. In such an embodiment, each hairpin barcode forward primer 10 comprises, from the 5′ end 10A to the 3′ end 10B, the adapter sequence 11, a UMI sequence comprising a structured UMI 14, and the target-specific sequence 16 complementary to a respective portion 2 of a TCR or BCR clonotype. Correspondingly, each hairpin barcode reverse primer comprises, from the 5′ end 20A to the 3′ end 20B, the adapter sequence 21, a UMI sequence comprising a structured UMI 24, and the target-specific sequence 26 complementary to a respective portion 3 of a TCR or BCR clonotype. At least a portion 12, 22 of the adapter sequence 11, 21 of the hairpin barcode forward primer 10 and/or the hairpin barcode reverse primer 20 is complementary to at least a portion 15, 25 of the UMI sequence 17, 27 of the hairpin barcode forward primer 10 and/or the hairpin barcode reverse primer 20. The adapter sequence 11, 21 and the UMI sequence 17, 27 are configured to hybridize to each other at or under a closed annealing temperature and not hybridize to each other at or above an open annealing temperature.

The method as shown in FIG. 2 also comprises amplifying, in step S11, the nucleic acid molecules 1 by performing PCR pre-amplification of the nucleic acid molecules 1 to form a plurality of barcoded PCR products 50. The PCR pre-amplification as performed in step S11 has an annealing temperature equal to or less than the closed annealing temperature of the hairpin barcode forward primers 10 and/or the hairpin barcode reverse primers 20.

The method further comprises contacting, in step S12, the plurality of barcoded PCR products 50 with an adapter-specific forward primer 30 and an adapter-specific reverse primer 40 and amplifying, in step S13, the barcoded PCR products 50 by performing PCR amplification on the barcoded PCR products 50 to form a library of amplified barcoded PCR products 60. In an embodiment, at least a portion of cycles of the PCR amplification has an annealing temperature greater than or equal to the open annealing temperature of the hairpin barcode forward primers 10 and/or the hairpin barcode reverse primers 20.

The method also comprises sequencing, in step S14, at least a respective portion of the amplified barcoded PCR products 60 to form respective sequence reads comprising the UMI(s) 64 and TCR or BCR sequence(s) 66. The method further comprises demultiplexing, in step S15, the sequence reads based on nucleic acid sequences of the UMIs 64 and mapping, in step S16, the demultiplexed sequence reads to respective TCR or BCR clonotypes based on nucleic acid sequences of the TCR or BCR sequences 66. The lymphocyte clonality for the sample is then determined in step S17 based on the demultiplexed and mapped sequence reads.

The sample used in the determination of lymphocyte clonality could be any sample comprising nucleic acid molecules 1 of lymphocytes, such as T cells or lymphocytes, B cells or lymphocytes or T and B cells or lymphocytes.

In an embodiment, the sample is an enriched lymphocyte sample, in which lymphocytes have been enriched and isolated from a biological sample, such as taken from a subject. For instance, lymphocytes could be enriched from buffy coats or peripheral blood mononuclear cells (PBMCs) using immunomagnetic cell separation, fluorescence-activated cell sorting (FACS) or other lymphocyte enriching techniques.

However, an advantage of the present invention is that no lymphocyte enrichment is needed in order to determine lymphocyte clonality. For instance, PBMCs could be isolated from buffy coat of a subject and used as sample in the method shown in FIG. 2.

The sample could therefore be a lymphocyte enriched sample or a biological sample that is not subject to lymphocyte enrichment. Examples of the latter include a blood sample or a buffy coat sample. The buffy coat is the fraction of an anticoagulated blood sample that contains most of the lymphocytes and platelets following density gradient centrifugation. In fact, the present invention can be used for any sample comprising lymphocytes or lymphocyte nucleic acid molecules, also including tissue samples, a fixed sample, i.e., a biological sample, such as a tissue sample, that has been subject to fixation to preserve the biological sample.

True clonal variation is difficult to separate from sequencing errors. By comparing the frequency of multiple clonotypes that differ from each other by merely one or a few nucleotides, many clonality determining methods that are based on bioinformatical processes assume that low-frequency clonotype(s) is(are) a sequencing mistake and instead interpret such a low-frequency clonotype as being one of the more frequently expressed clonotypes but with one or a few sequencing errors. Hence, a sample containing a low-frequency clonotype and a high-frequency clonotype may in such a case be misinterpreted to only contain the high-frequency clonotype.

Furthermore, amplification steps in the clonality determination introduce PCR-induced amplification biases and errors, which effectively prevent accurate clonality determination and quantification. UMIs are known to be used to reduce such PCR-induced amplification biases and errors. To date, UMIs have mostly been applied to messenger ribonucleic acid (mRNA) when profiling the immune receptor repertoire of B and T cells to remove PCR duplicates and improving sequence accuracy. However, the analysis of mRNA has several disadvantages. Firstly, the number of transcripts per cell is not constant. Hence, the correct number and size of various T or B cell clones cannot be accurately estimated. Secondly, the reverse transcription efficiency is variable between sequences, and reverse transcriptase is up to 1000 times more prone to introduce errors than DNA polymerase, causing both quantification and sequence biases. Hence, the nucleic acid molecules 1 are DNA molecules 1 and the sample comprises DNA molecules 1 of lymphocytes.

Immune repertoire sequencing can be applied to both T and B cells to explore a V(D)J recombination (Clinical Immunology (2020) 66(9): 1228-1237). In another application, ultrasensitive immune repertoire sequencing is used to explore the repertoire of the recombined immunoglobulin heavy (IGH) locus. Using a set of primers specific for all the IGH variable regions and a different set of primers specific for all IGH joining regions (Table 1) an immune repertoire can be amplified and analyzed using ultrasensitive immune repertoire sequencing. In this specific implementation, the primer containing the hairpin with barcode is the primer complementary to the J region. In the specific implementation, the primers are not perfectly complementary to the variable or joining region as each primer can amplify a plurality of variable or joining regions with share homology respectively, see FIG. 17.

TABLE 1
primers for immune repertoire sequencing of the IGH locus

| Name | Sequence 5′-3′ | SEQ ID NO: |
|---|---|---|
| J-h- | GGACACTCTTTCCCTACACGACGCTCTTCCGATCT-UMI- | 91 |
| consensus_rev | ATGGGAAAGAGTGTCCCTTACCTGAGGAGACGGTGACC | |
| VH1-FR3_fw | GTGACTGGAGTTCAGACGTGTGCTCTTCCGATCTTGGAGCTGAGCAGC | 92 |
| | CTGAGATCTGA | |
| VH2-FR3_fw | GTGACTGGAGTTCAGACGTGTGCTCTTCCGATCTCAATGACCAACATG | 93 |
| | GACCCTGTGGA | |
| VH3-FR3_fw | GTGACTGGAGTTCAGACGTGTGCTCTTCCGATCTTCTGCAAATGAACA | 94 |
| | GCCTGAGAGCC | |
| VH4-FR3_fw | GTGACTGGAGTTCAGACGTGTGCTCTTCCGATCTGAGCTCTGTGACCG | 95 |
| | CCGCGGACACG | |
| VH5-FR3_fw | GTGACTGGAGTTCAGACGTGTGCTCTTCCGATCTCAGCACCGCCTACC | 96 |
| | TGCAGTGGAGC | |
| VH6-FR3_fw | GTGACTGGAGTTCAGACGTGTGCTCTTCCGATCTGTTCTCCCTGCAGC | 97 |
| | TGAACTCTGTG | |
| VH7-FR3_fw | GTGACTGGAGTTCAGACGTGTGCTCTTCCGATCTCAGCACGGCATATC | 98 |
| | TGCAGATCAG | |

UMI as referred to in Table 1 could be any structured UMI 14 as disclosed herein.

In an embodiment, step S17 of FIG. 2 comprises determining the lymphocyte clonality for the sample based on the demultiplexed and mapped sequence reads in step S17. In an embodiment, this step S17 comprises identifying any TCR or BCR clonotypes present in the sample. Hence, this step S17 preferably comprises identifying all TCR or BCR clonotypes that are present in the sample to thereby identify individual T cell or B cell clones in the sample. In an embodiment, step S17 also, or additionally, comprises quantifying the TCR or BCR clonotypes present in the sample based on the demultiplexed and mapped sequence reads.

Hence, not only the identities of the different TCR or BCR clonotypes in the sample are determined in step S17 but also a quantity thereof. Such a quantification of TCR or BCR clonotypes is possible due the presence of structured UMI 14, 24 in the hairpin barcode forward primers 10 and/or the hairpin barcode reverse primers 20. The quantification can be an absolute quantification, i.e., the total copy number of each TCR or BCR clonotype present in the sample, or a relative quantification, e.g., frequencies of the different TCR or BCR clonotypes present in the sample such as relative to each other or a reference sequence present in or added to the sample or the reaction mixtures in any of the steps S11 to S13.

In a particular embodiment, quantifying the nucleic acid molecules, such as TCR or BCR clonotypes, comprises quantifying the nucleic acid molecules, such as TCR or BCR clonotypes, based on determining the number of different UMIs 64, optionally with at most a first predefined number mismatches, having the same nucleotide sequence of the nucleic acid molecule sequences, such as TCR or BCR sequence 66, optionally with at most a second predefined number of mismatches.

In a particular embodiment, the first predefined number is zero, one or two, preferably zero or one, and the second predefined number is an integer equal to or larger than 0 but no larger than ten, preferably no larger than eight and more preferably no larger than five.

For instance, the Molecular Identifier Guided Error Correction (MIGEC) pipeline could be used in the demultiplexing and mapping steps S15 and S16. MIGEC comprises de-multiplexing, adapter trimming and read overlapping for sequence reads data and extraction of UMI sequences. MIGEC also comprises assembly of consensuses for original molecules that entered library preparation by grouping reads with identical molecular identifiers and mapping of V, D and J segments, extraction of CDR3 regions and clonotype assembly for all human and mouse immune receptors (TRA/TRB/TRG/TRD and IGH/IGL/IGK). Further features of MIGEC include additional filtering of hot-spot errors in CDR3 sequence.

In an embodiment, if P is one then Q is equal to or larger than two and if Q is one then P is equal to or larger than two. In this particular embodiment, multiple different forward primer 10 and/or multiple different reverse primers 20 are used in the method.

In an embodiment, determining lymphocyte clonality comprises determining clonality of αβ TCRs and αβ T cells. In a particular embodiment, the P forward primers 10 comprise at least one forward primer 10 per V region of a TCR alpha (α) chain and the Q reverse primers 20 comprise at least one reverse primer 20 per J region of the TCR alpha (α) chain. In other particular embodiments, the P forward primers comprise at least one forward primer 10 per V region of a TCR beta (β) chain and the Q reverse primers 20 comprise at least one reverse primer 20 per D region of the TCR beta (β) chain, or the P forward primers 10 comprise at least one forward primer 10 per D region of the TCR beta (β) chain and the Q reverse primers 20 comprise at least one reverse primer 20 per J region of the TCR beta (β) chain, or the P forward primers 10 comprise at least one forward primer 10 per V region of the TCR beta (β) chain and the Q reverse primers 20 comprise at least one reverse primer 20 per J region of the TCR beta (β) chain.

It is also possible to determine clonality of both the TCR alpha (α) chain and the TCR beta (β) chain in the same sample. In such a case, combinations of forward and reverse primers 10, 20 according to above are used in the same reaction mixture, or the sample is split into two samples and the two clonality determinations are run in parallel.

In another embodiment, determining lymphocyte clonality comprises determining clonality of γδ TCRs and γδ T cells. In a particular embodiment, the P forward primers 10 comprise at least one forward primer per V region of a TCR gamma (γ) chain and the Q reverse primers 20 comprise at least one reverse primer 20 per J region of the TCR gamma (γ) chain. In other particular embodiments, the P forward primers 10 comprise at least one forward primer 10 per V region of a TCR delta (δ) chain and the Q reverse primers 20 comprise at least one reverse primer 20 per D region of the TCR delta (δ) chain, or the P forward primers 10 comprise at least one forward primer 10 per D region of the TCR delta (δ)) chain and the Q reverse primers 20 comprise at least one reverse primer 20 per J region of the TCR delta (δ) chain, or the P forward primers 10 comprise at least one forward primer 10 per V region of the TCR delta (δ) chain and the Q reverse primers 20 comprise at least one reverse primer 20 per J region of the TCR delta (δ) chain.

It is also possible to determine clonality of both the TCR gamma (γ) chain and the TCR delta (δ) chain in the same sample. In such a case, combinations of forward and reverse primers 10, 20 according to above are used in the same reaction mixture, or the sample is split into two samples and the two clonality determinations are run in parallel.

In fact, it is actually possible to determine clonality of both αβ TCRs and αβ T cells and γδ TCRs and γδ T cells in the same sample according to the present invention.

In a further embodiment, determining lymphocyte clonality comprises determining clonality of BCRs and B cells. In particular embodiments, the P forward primers 10 comprise at least one forward primer 10 per V region of the BCR and the Q reverse primers 20 comprise at least one reverse primer 20 per D region of the BCR, or the P forward primers 10 comprise at least one forward primer 10 per D region of the BCR and the Q reverse primers 20 comprise at least one reverse primer 20 per J region of the BCR, or the P forward primers 10 comprise at least one forward primer 10 per V region of the BCR and the Q reverse primers 20 comprise at least one reverse primer 20 per J region of the BCR.

In fact, it is actually possible to determine clonality of both αβ and/or γδ TCRs and αβ and/or γδ T cells and BCRs and B cells in the same sample according to the present invention.

In a particular embodiment, the P forward primers 10 are multiple different hairpin barcode forward primers 10 comprising at least one hairpin barcode forward primer 10 per variable (V) region of a TCR or BCR. In such a particular embodiment, each target-specific sequence 16 of the hairpin barcode forward primers 10 is preferably complementary to a respective variable (V) region 2 of the TCR or BCR.

In such an embodiment, step S10 of FIG. 2 preferably comprises contacting the sample with the multiple different hairpin barcode forward primers 10 and multiple different reverse primers 20. The multiple different reverse primers 20 comprise at least one reverse primer 20 per joining (J) region 3 of the TCR or BCR. In this embodiment, each reverse primer 20 comprises, from the 5′ end 20A to the 3′ end 20B, the adapter sequence 23 and a target-specific sequence 26 complementary to a respective joining (J) region 3 of the TCR or BCR.

The monitoring of T and/or B cell clonality has immense importance in the diagnosis and follow-up of various disease, such as hematological diseases and is a fundamental tool for the understanding of antigen specificity in immunity to infections, cancer, and in autoimmunity. Clinically, there is a need for improved methodological specificity and sensitivity to determine the immune repertoire, for example, to detect acute lymphoblastic leukemia and to monitor minimal residual disease. Moreover, in studies of immune disorders such as autoimmunity and tumor immunity, improved immune repertoire analysis will facilitate the identification of involved immune cells, be used to evaluate the efficiency of immunomodulating drugs, and explore their mode of action.

Thus, the lymphocyte clonality as determined according to the present invention is a valuable information source in diagnosis and monitoring of various diseases, and can thereby be used in disease characterization.

Another aspect of the invention therefore relates to a method of disease characterization. The method comprises determining lymphocyte clonality as disclosed herein of biological sample comprising nucleic acid molecules 1 of lymphocytes obtained from a subject. The method also comprises characterizing a disease of the subject based on the determined lymphocyte clonality. In an embodiment, the disease is selected from the group consisting of a hematologic disease, an infectious disease, a cancer disease and an autoimmune disease.

Characterizing a disease as used herein could, in an embodiment, include predicting or determining a likelihood that the subject is suffering from the disease. Hence, information of the lymphocyte clonality, as determined from a sample taken from the subject, can be used to assess whether the subject is likely to suffer from the disease.

For instance, information of lymphocyte clonality could be useful in monitoring infections, such as bacterial infections or virus infections. Hence, in an embodiment, information of lymphocyte clonality as determined according to the invention could be used in monitoring, including diagnosing, a virus infection, such as a coronavirus infection, including, but not limited to, a severe acute respiratory syndrome (SARS) coronavirus 1 (SARS-CoV-1), SARS-CoV-2, Middle East Respiratory Syndrome (MERS) coronavirus (MERS-CoV).

Alternatively, or additionally, characterizing a disease could comprise monitoring disease progression, such as monitoring whether the subject recovers from a disease, whether the subject's condition deteriorates or whether subject's disease condition remains unchanged. Thus, in information of lymphocyte clonality could be used to monitor disease progression and recovery from the disease.

Characterizing a disease could further include evaluating the efficiency of various treatments of a disease, such as administration of drugs, e.g., immunomodulating drugs, and/or determining their mode of action. Hence, information of lymphocyte clonality could then be used to assess how well a subject responds to the treatment and whether the treatment seem to have any effect on the disease or, whether another potentially more effect treatment should be selected instead.

### EXAMPLES

### Example 1

This Example investigated whether formation of non-specific PCR product caused by UMIs can be minimized by adding specific nucleotides within the UMIs, i.e., structured UMIs, to reduce the possibility of stable interactions. Here, we developed a digital sequencing approach based on the simple multiplexed PCR based barcoding of DNA for ultrasensitive mutation detection using next-generation sequencing (SiMSen-Seq) technology. Assay performance of several structured UMI strategies was evaluated. Assay characteristics and specificity were analyzed by quantitative PCR, capillary gel electrophoresis, melting curve analysis and sequencing. We demonstrate how both individual assays and multiplexed panels are improved using structured UMIs. Improved experimental protocols to mitigate the limitations of UMIs will facilitate the implementation of digital sequencing in clinical and basic research settings.

### Materials and Methods

### Library Construction

SiMSen-Seq assays were designed and tested as previously described (Nat. Protoc. (2017) 12: 664-682). The SiMSen-Seq protocol consists of two PCR steps followed by library purification and sequencing. The first 10 μl barcoding PCR contained, 0.05 U Platinum SuperFi I DNA polymerase, 1× SuperFi Buffer (#12351010, Thermo Fisher Scientific), 0.2 mM deoxyribonucleotide triphosphate (#D7295, Merck), 40 nM of each primer (Ultramer, Integrated DNA Technologies, Table 5), 0.5 M L-carnitine inner salt (#C0158, Sigma-Aldrich) and 20 ng human genomic DNA (#11691112001, Roche Diagnostics). The standard curve was performed with 27, 10, 3, 1 and 0.33 ng DNA per reaction. The following thermal program was used on a T100 Thermal cycler (Bio-Rad Laboratories): 30 s at 98° C., 3 cycles of amplification (98° C. for 10 s, 62° C. for 6 min, 72° C. for 30 s), 15 min at 65° C., and 15 min at 95° C. At the start of the 15 min incubation step at 65° C., the DNA polymerase was inactivated by adding 20 μl TE-buffer (pH 8.0, #AM958, Thermo Fisher Scientific) supplemented with 45 ng/μl Streptomyces griseus protease (#P5147-100MG, Merck). The second adapter PCR was performed in 40 μl reactions, containing 1×Q5 Hot Start High-Fidelity Master Mix (#M0494S, New England BioLabs) and 400 nM of each Illumina adapter primer (desalted, Sigma-Aldrich, Table 2). The following thermal program was used on a T100 Thermal cycler: 98° C. for 3 min, followed by 30 cycles of amplification (98° C. for 10 s, 80° C. for 1 s, 72° C. for 30 s, 76° C. for 30 s, all with ramping rate at 0.2° C./s). The 16-plex was experimentally performed as an 18-plex reaction, but two assays (TP53_C and TP53_D) were omitted in downstream analysis, since they have overlapping amplicons.

### Sequencing

Library concentrations were assessed using on a 5200 Fragment Analyzer (Agilent Technologies) using the DNF-474 HS NGS Fragment Kit (1-6000 base pairs, #DNF-474-1000, Agilent Technologies). Samples were pooled to enable similar sequencing depth across all samples. The pooled library was purified with a Pippin Prep (Sage Science) using 2% agarose, dye-free, Pippin Gel Cassette (100-600 base pairs, #CDF2010, Sage Science), according to manufacturer's instructions. Libraries were collected within the 205-305 bp size range. Purified libraries were quantified using quantitative PCR and the NEBNext Library Quant Kit (#E7630, New England Biolabs). Briefly, 2 μl of the purified libraries was quantified in 10 μl reactions, containing 1×TATAA SYBR GrandMaster Mix Low Rox (#TA01-3750LR, TATAA Biocenter) and 400 nM of each ILLUMINA® adapter primer (SEQ ID NO: 6 and 8). The concentration was estimated using a standard curve with adjustment to the average library size. Sequencing was performed on a MiniSeq (Illumina) with 20% PhiX (#FC-110-3001, Illumina) using the MiniSeq High Output Reagent Kit (#FC-420-1002, Illumina). The final library concentration was 1.4 pM and sequencing was performed in single-end 150 base pairs mode.

### Data Analysis

Sequencing data was analyzed using the UMIErrorCorrect pipeline (version 0.21, https://github.com/stahlberggroup/umierrorcorrect). Briefly, fastq-files were preprocessed by trimming off the UMI and spacer sequence. The pipeline then aligned reads to the hg38 reference genome using Burrows-Wheeler aligner (Bioinformatics (2009) 25: 1754-1760), clustering reads into UMI families based on their UMI sequence and chromosomal position. Each UMI family was collapsed to one consensus read, counting the most common nucleotide type at each position. Final data was filtered using UMI family size cutoff ≥3. The number of off-target reads was calculated as the difference between the total number of observed reads and the number of reads aligned to intended target sequences. The error rate was calculated as the total number of non-reference alleles per nucleotide position divided by coverage for each nucleotide position. Then the average of all nucleotide positions was calculated per amplicon. Single nucleotide polymorphism positions identified in the Single Nucleotide Polymorphism Database of Nucleotide Sequence Variation were excluded (Nucleic Acids Res (2001) 29(1): 308-311).

Figures and statistical tests were performed using GraphPad Prism version 8.4.3 (GraphPad Software) and R version 4.2.2 (R Core Team. R: A language and environment for statistical computing. Vienna, Austria: R Foundation for Statistical Computing; 2022.), using packages ggplot2 (Wickham H. ggplot2: elegant graphics for data analysis. New York: Springer-Verlag; 2016.) and ggpubr (Kassambara A. ggpubr: “ggplot2” based publication ready plots. CRAN; 2020).

### Quantitative PCR

The amount of UMI-tagged DNA was quantified using quantitative PCR and a CFX384 Touch Real-Time PCR Detection System (Bio-Rad Laboratories). Two microliters PCR product from the first barcoding PCR reaction was analyzed in 10 μl reactions, containing 1×TATAA SYBR GrandMaster Mix Low Rox (#TA01-3750LR, TATAA Biocenter) and 400 nM of each Illumina adapter primer (Table 5). The following thermal program was used: 98° C. for 3 min, 45 cycles of amplification (98° C. for 10 s, 80° C. for 1 s, 72° C. for 30 s, 76° C. for 30 s, all with ramping rate at 0.2° C./s) and melting curve analysis ranging from 60° C. to 95° C., 0.5° C./s increments. Cycle of quantification values were determined by regression using the Bio-Rad CFX Maestro software (version 4.1, Bio-Rad Laboratories).

### Capillary Gel Electrophoresis

The PCR products from the second adapter PCR were evaluated on a 5200 Fragment Analyzer using dsDNA 915 Reagent kit (35-5000 base pairs, #DNF-915-K1000, Agilent Technologies), according to the manufacturer's instructions. Library purity was calculated as the fraction of specific library product (200-300 base pairs) compared with all formed PCR products. Analysis was performed in ProSize Data Analysis Software version 4.0.0.3 (Agilent Technologies).

### Melting Curve Analysis

The melting temperature of the stem-loop structure in barcoding primers was determined by high resolution melting curve analysis using a Mic PCR machine (Bio Molecular Systems). The 20 μl reaction contained 1× SuperFi buffer, 1×SYBR Green I nucleic acid gel stain (#S9430, Sigma-Aldrich) and 1 μM forward barcoding primer (Table 5). The temperature profile was 95° C. for 3 min, 60° C. for 3 min, followed by melting analysis from 60° C. to 90° C. with ramping at 0.03° C./s. Melting curve analysis was performed using the Mic quantitative PCR software, version 2.10.03 (Bio Molecular Systems).

TABLE 2
Primer sequences

| | | SEQ | | SEQ | |
|---|---|---|---|---|---|
| Assay | | ID | | ID | |
| name | F primer (5′→3′) | NO: | R primer (5′→3′) | NO: | 3-plex |
| ARID2 | TGCTCATTTTGAAGTAAATCCAG | 10 | ACTGCAAGTCGAGAGGTATTCAG | 11 | H |
| | ATTGTTCTGTT | | AATACATT | | |
| BRAF | TGGGACCCACTCCATCG | 12 | ATATTTCTTCATGAAGACCTCAC | 13 | G |
| | | | AGTAA | | |
| CDKN2A | CAACTGCGCCGACCCC | 14 | CAGCGTGTCCAGGAAGCC | 15 | E |
| CNOT9 | TTGCACACTGTCAGCAAAAC | 16 | ACCAATAACTCCAAGGCTGGT | 17 | I |
| CTNNB1 | GTCACTGGCAGCAACAGTCTTAC | 18 | TTCCTCAGGATTGCCTTTACCAC | 19 | A |
| | CTG | | TCAGA | | |
| CYSLTR2 | TGGCCTGCAGGATTATGTCTTAT | 20 | CCAGGAAACGCACAACACTCAG | 21 | J |
| | T | | | | |
| EZH2 | CTGAATACAGGTTATCAGTGCCT | 22 | TCAAAGATCCTGTGCAGAAAAAT | 23 | M |
| | | | GAAT | | |
| FBXW7_A | TGCAGAGTTCAGTTACCTTAGGA | 24 | TGCATACATCAGACAGCACAGA | 25 | F |
| | GA | | | | |
| FBXW7_B | CCATGACAAGATTTTCCCTTACC | 26 | CACCTTATATGGGCATACTTCCA | 27 | L |
| | | | CT | | |
| GNA11_A | CCACCTTGGGCTACCTGC | 28 | GGTACTCGATGATGCCGGTG | 29 | D |
| GNA11_B | GTCCTTTCAGGATGGTGGATGTG | 30 | GGATGTCACGTTCTCAAAGCAGT | 31 | C |
| GNAQ_A | ATTCGATGATCCCTGTGGTGG | 32 | GTAGCTGACCCTGCCTACCT | 33 | M |
| GNAQ_B | GCAGTGTATCCATTTTCTTCTCT | 34 | GTATTGTTAACCTTGCAGAATGG | 35 | L |
| | CT | | TC | | |
| HRAS | GCGATGACGGAATATAAGCTG | 36 | CTGGATCAGCTGGATGGTCA | 37 | F |
| IDH1_A | CCAACATGACTTACTTGATCCCC | 38 | CTTGTGAGTGGATGGGTAAAACC | 39 | H |
| | ATAA | | TA | | |
| IDH2_B | AGTGGATCCCCTCTCCACC | 40 | TAGTCCCTGGCTGGACCAAG | 41 | M |
| MEK1_C | AGGGAGCTGCAGGTTCTG | 42 | GCCATCGCTGTAGAACGCA | 43 | E |
| MEK1_D | GCAGATGTCAAGCCCTCCAA | 44 | GCTGACCCCAAAGTCACAGA | 45 | C |
| MEK1_A | CGCCTTGAGGCCTTTCTTAC | 46 | GTCGTCATCCTTCAGTTCTCC | 47 | D |
| MEK1_B | AACCTCTCTTTCTTCCACCTTTC | 48 | CTTATGATCTGGTTCCGGATTGC | 49 | G |
| | TC | | G | | |
| NF1 | TCATCAGTTCCTCCTCAGAATTC | 50 | AAGCATACCTGGTATAAACAGTG | 51 | B |
| | CCCC | | GCACAC | | |
| NRAS | GCTGGATTGTCAGTGCGCTTTTC | 52 | GACTGAGTACAAACTGGTGGTGG | 53 | J |
| | C | | TTGG | | |
| PLCB4 | CGCATTTACCCCAAGGGAGG | 54 | CAGCCAGCGTTCCAGAAAATC | 55 | A |
| PPP6C | AGCCTTCGTGCACTAGTTGAT | 56 | GTGCCTTTCTAAATTGCAGTTTG | 57 | B |
| | | | TT | | |
| RAC1 | ACTTGCCTACTGATCAGTTACAC | 58 | GGTTAATTCCCAATTTAAGATAC | 59 | J |
| | AACCAATG | | TTACACAGTAGGG | | |
| SF3B1_F | GGCTCCCATAACTGCCTGAAT | 60 | GGATTTGGTTGTGAAGATTCGCT | 61 | E |
| SF3B1_A | CCATGTAAGTCCTAGCATTCATT | 62 | TGTACTCTTTTTCCTGTTGCTGT | 63 | E |
| | T | | | | |
| SF3B1_B | GAGGCTACAACAGCAAAAGCTC | 64 | CTATGATCTCTACCATGAGACCT | 65 | L |
| | | | GA | | |
| SF3B1_C | AGATGGCACAGCCCATAAGAA | 66 | GAAGTCCTGGCAAGCGAGA | 67 | D |
| SF3B1_D | GCAATGGCCAAAGCACTGAT | 68 | CCTGTGTTTGGTTTTGTAGGTCT | 69 | D |
| SF3B1_E | ACCATCAATCAGTTGTTCTTCAA | 70 | AAATGGTGATGGAGACAATTGAG | 71 | H |
| | GT | | | | |
| STK19 | GTACGTCACTGCTCTGCGCCG | 72 | GGTAGGAATGGAGGGAAGAGAAC | 73 | B |
| | | | CTGAA | | |
| TP53_D | GTGGCAAGTGGCTCCTGA | 74 | CATGGGCGGCATGAAC | 75 | C |
| TP53_A | GGCCCCTGTCATCTTCTGT | 76 | CAGAATGCAAGAAGCCCAGA | 77 | A |
| TP53_B | TTGCGTGTGGAGTATTTGGA | 78 | AGACCTCAGGCGGCTCATAG | 79 | I |
| TP53_C | CTTCCAGTGTGATGATGGTGAGG | 80 | ACATGTGTAACAGTTCCTGCATG | 81 | G |
| | ATGG | | GGC | | |
| TP53_E | TCCTCTGTTGCTGCAGATCC | 82 | TGAGTTCCAAGGCCTCATTC | 83 | I |
| WT1 | CCTGGTGTGGGTCTTCAGGTGG | 84 | GTGTGAAACCATTCCAGTGTAAA | 85 | F |
| | | | ACTTGTCAG | | |
| TP53_F | GTGGCAAGTGGCTCCTGA | 86 | CATGGGCGGCATGAAC | 87 | G |

Additional sequence of forward (F) primer (SEQ ID NO: 9):

GGACACTCTTTCCCTACACGACGCTCTTCCGATCTNNNNNNNNNNNNAT
GGGAAAGAGTGTCC

Additional sequence of reverse I primer (SEQ ID NO: 5):

GTGACTGGAGTTCAGACGTGTGCTCTTCCGATCT

TABLE 3
Barcode sequences

| Name | Sequence | SEQ ID NO: |
|---|---|---|
| A | NNNNNWWNNNNN | — |
| B | SSWSWWWSWWSWSS | 88 |
| C | SWSSWWWSSWSWWWSWWSWSS | 89 |
| D | NNANNNNANNNNANN | — |
| E | NNANNANNANNANNANN | — |
| F | NNNNAAANNANNAAANNNN | — |
| G | NNNAANNNAANNNAANNN | — |
| H | NNTNNANNCANNANN | — |
| I | NNTNNANNCNNANN | — |
| J | NNNAANNNACANNNAANNN | — |
| K | NNTNNANNCNNANNTNN | — |
| L | NANTNANCNTNTNCNTNANTNAN | 90 |
| M | NANTNANCNTNTNCNTNAN | — |
| N | NNNNNNGAGCGNNNNNN | — |
| Reference | NNNNNNNNNNNN | — |

### Results

### Design of Different UMI Structures Using Digital Sequencing

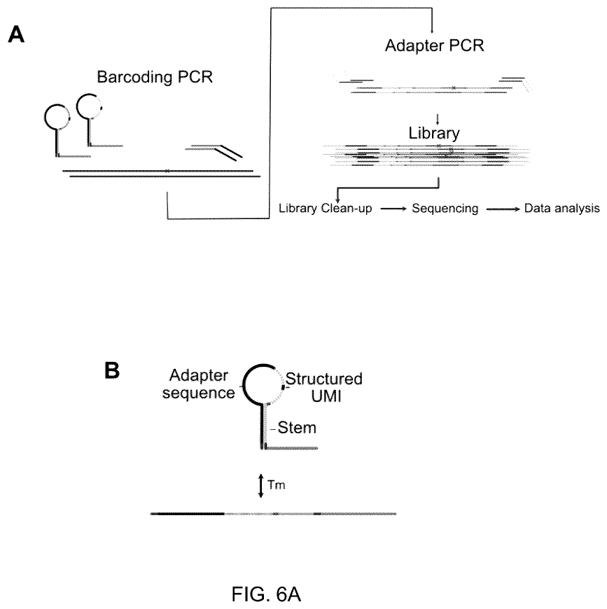

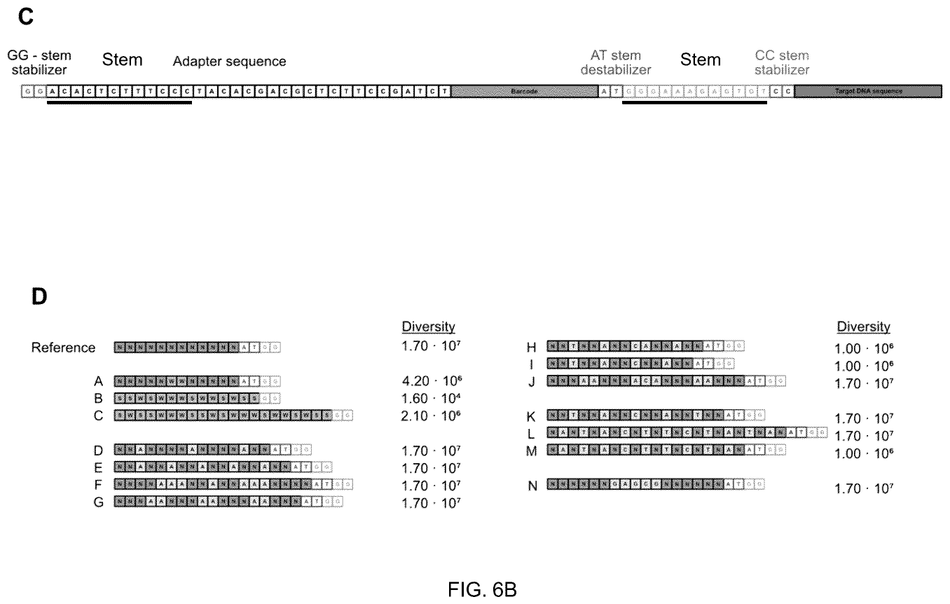

To evaluate different UMI structures, we used SiMSen-Seq that consists of two sequential PCR steps, i.e., barcoding and adapter PCR, followed by purification and sequencing (FIG. 6A). In the barcoding PCR step, primers with UMIs are used to label target DNA. In the subsequent adapter PCR step, barcoded target DNA is amplified by sequencing adapters. The rational of using SiMSen-Seq is that this approach already use three strategies to reduce formation of non-specific PCR products, including (i) the UMI is protected in a stem-loop structure that is closed during the annealing step in the first barcoding PCR, while open in the second adapter PCR (FIG. 61B), (ii) the barcoding primer concentration is limited and (iii) the barcoding PCR is terminated by an inactivation buffer containing protease. We designed and evaluated 14 different UMI structures, named A-N. These designs were compared with a reference UMI, consisting of a 12 nucleotide long and randomized sequence (FIGS. 6C-6D). All structured UMIs incorporate predefined nucleotides at specific positions within the reduced sequence, reducing their capacity to form stable internal structures and dimers. Eleven designs divided the UMIs into smaller segments of randomized nucleotides. Three designs (A, B and C) utilized degenerative nucleotides to reduce the risk of forming G-quadruplexes and internal stem structures. Four designs (D, E, F and G) used variable number and positions of adenine to split the UMI. Three designs (H, I and J) used different combinations of adenine and cytosine, while designs (K, L and M) used different combinations of all nucleotide types except guanine, and design (N) used a UMI structure with a combination of adenine, cytosine and guanine nucleotides. The number of possible UMI combinations for a given design is defined by the UMI length and type of nucleotides allowed. High UMI diversity is required for analysis of large amounts of DNA to reduce the risk that two target DNA molecules are labeled with identical UMI. Three designs (A, H and M) consisted of a shorter barcode, i.e., ten randomized nucleotides, reducing the diversity from 17 to 1.0 million combinations (FIG. 6D). The three designs that used degenerate nucleotides displayed a diversity of 4.2 million (A), 2.1 million (C) and 16,000 (B) combinations, respectively.

### Structured UMIs Improve Assay Performance

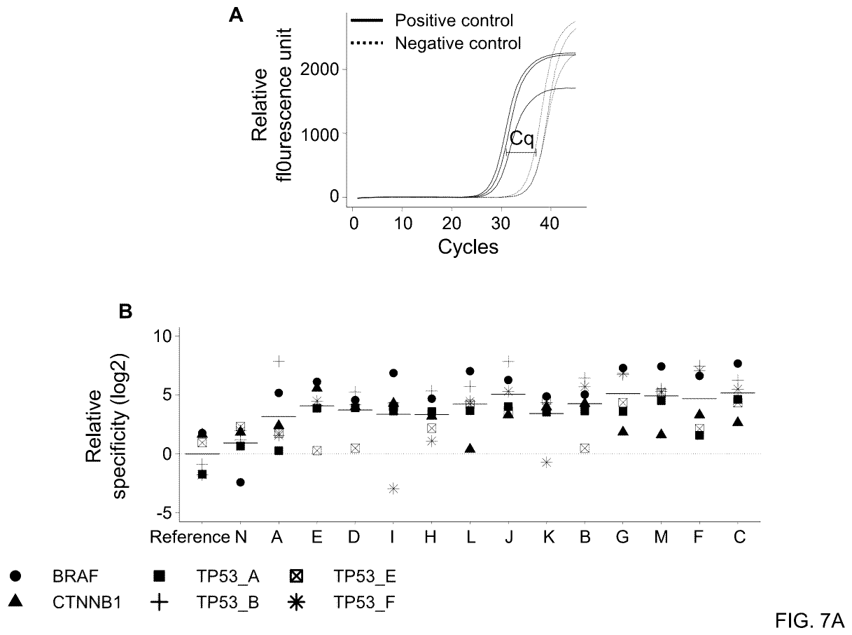

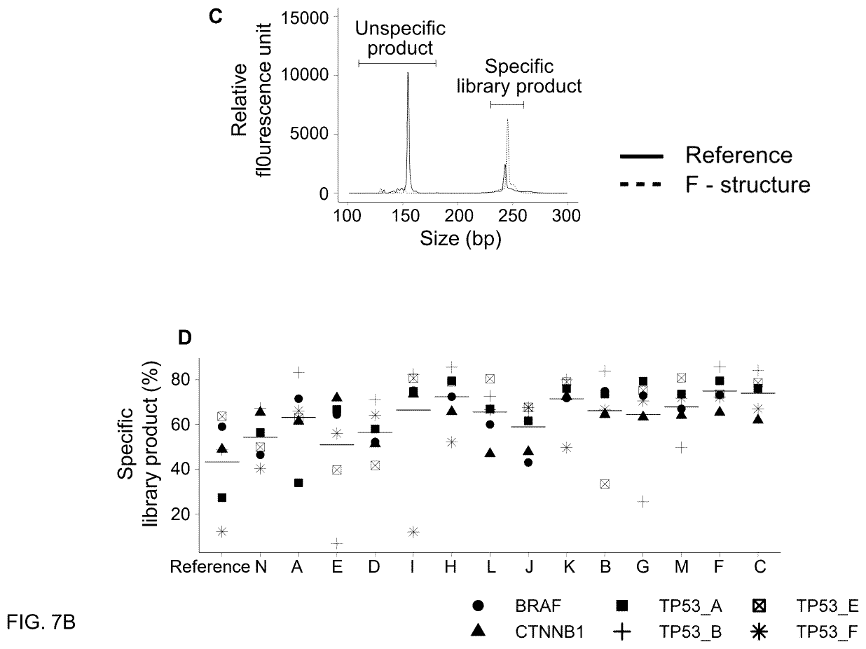

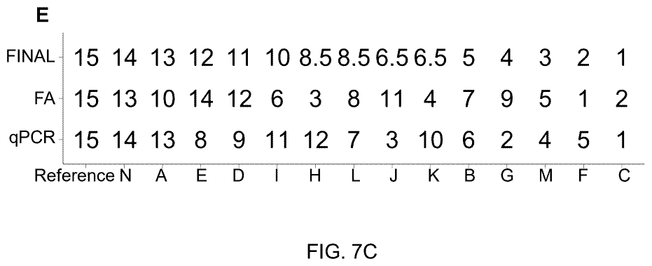

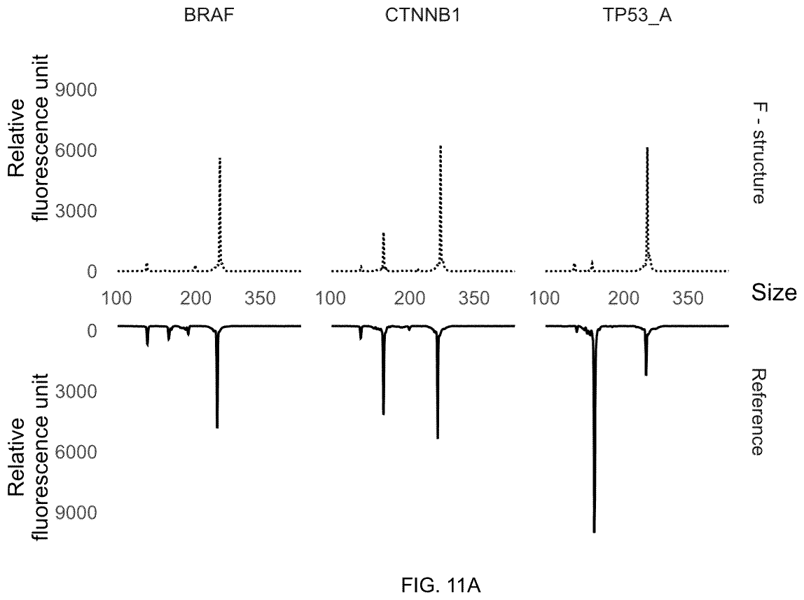

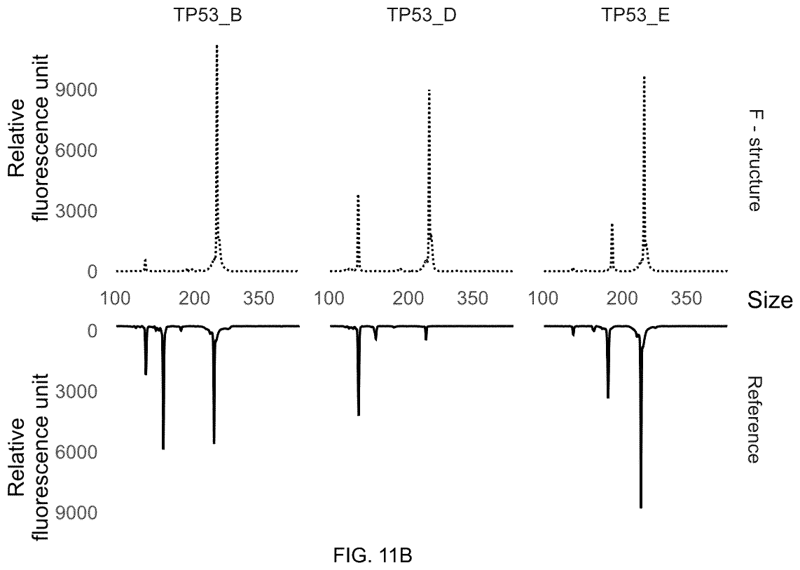

To evaluate assay performance of structured UMI designs, we performed the second adapter PCR as a quantitative PCR and compared data to the reference UMI (FIG. 7A). We used six different SiMSen-Seq assays and analyzed 20 ng human genomic DNA as template and water as negative controls. To compare the relative specificity between different structured UMIs we determined the differences in cycle of quantification values between DNA positive and negative samples, where large differences indicate superior performance (FIG. 7B). Overall, all structured UMI designs displayed improved specificity compared to the standard UMI. Design (C) performed best with 36 times higher specificity than reference UMI, followed by designs (F), (M) and (G). Next, we evaluated assay performance using capillary gel electrophoresis on final libraries (FIG. 7C). We quantified the amount of specific library relative to all generated PCR products. Again, all structured UMI designed were superior to the reference UMI (FIG. 7D and FIG. 11). Design (F) performed best with 31% more formed specific library compared with reference UMI, followed by designs (C), (H) and (K). To summarize the performance of different structured UMI designs, we ranked them in each evaluation test and summed up their rank score, providing a final ranking list (FIG. 7E). Structured UMI design (C) demonstrated best specificity followed by UMI designs (F), (M) and (G). Reduced UMI diversity (A, B, C, H, I and M) showed minor/no general improvements in assay performance compared to designs with maximal UMI diversity. Hence, we selected UMI design (F) for further downstream analysis, since it displayed higher complexity than UMI design (C).

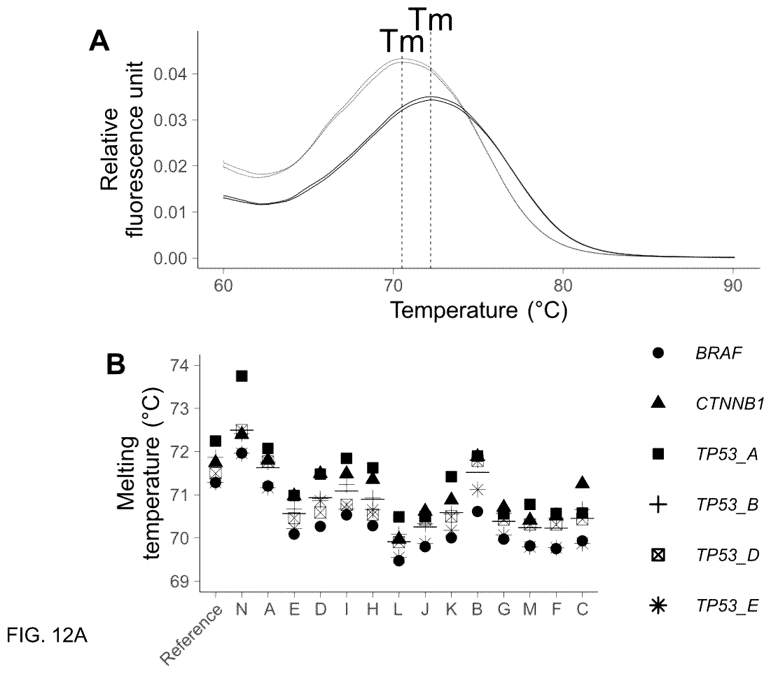

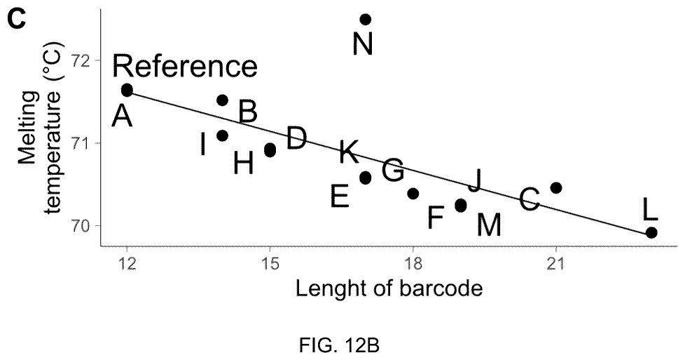

An important feature in the SiMSen-Seq primer design to reduce formation of non-specific PCR products is that the stem structure is closed during the annealing step in barcoding PCR, while open during the annealing step in following adapter PCR (FIGS. 6A-6B). Melting curve analysis of forward barcoding PCR primer showed that most UMI designs displayed a somewhat lower melting temperature compared to reference UMI, which is caused by the extra nucleotides added to the loop structure (FIGS. 12A-12B). The melting temperature correlated to the nucleotide length of the loop structure (FIG. 12C), but remained ≥8° C. above the annealing temperature of the barcoding PCR, thus ensuring a closed stem structure.

### Validation of Improved Assay Performance Using Structured UMIs

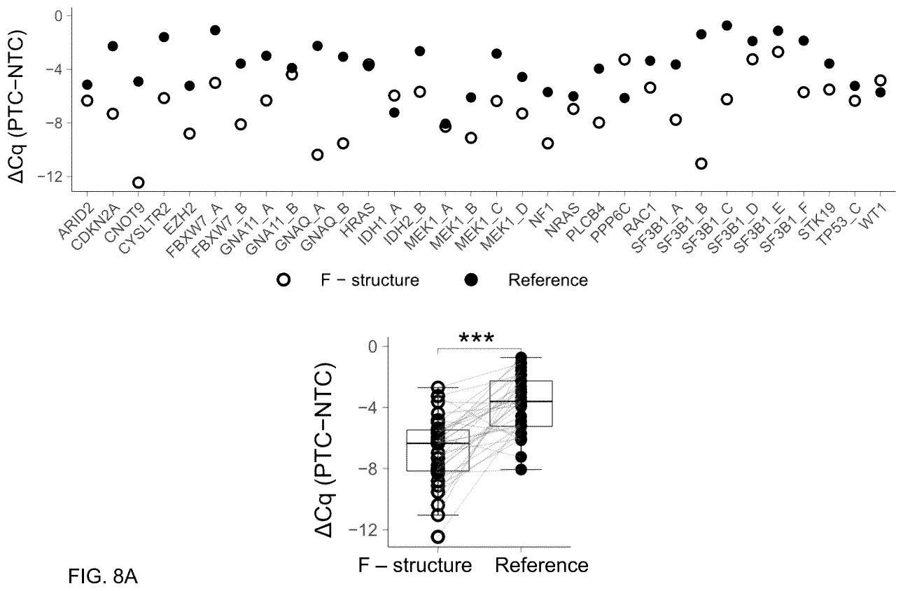

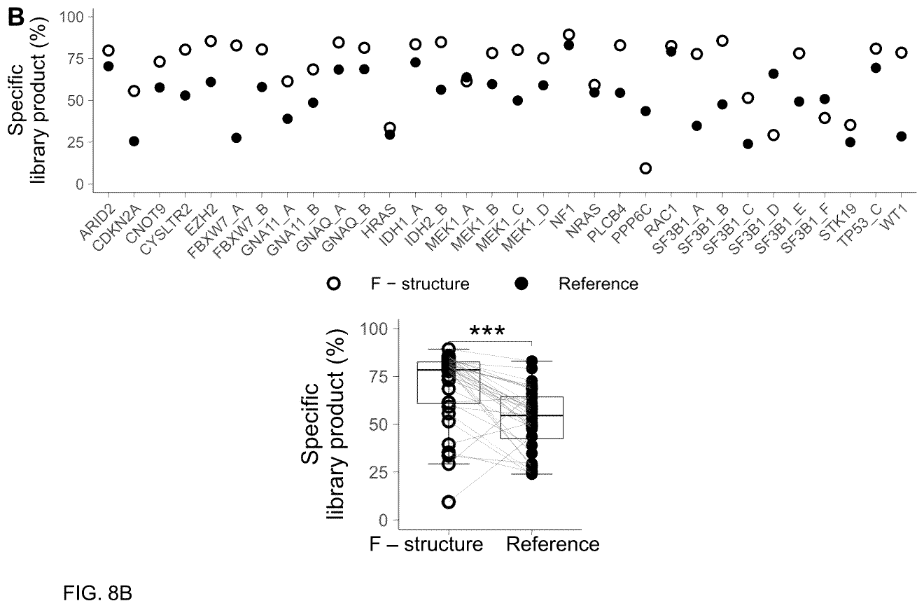

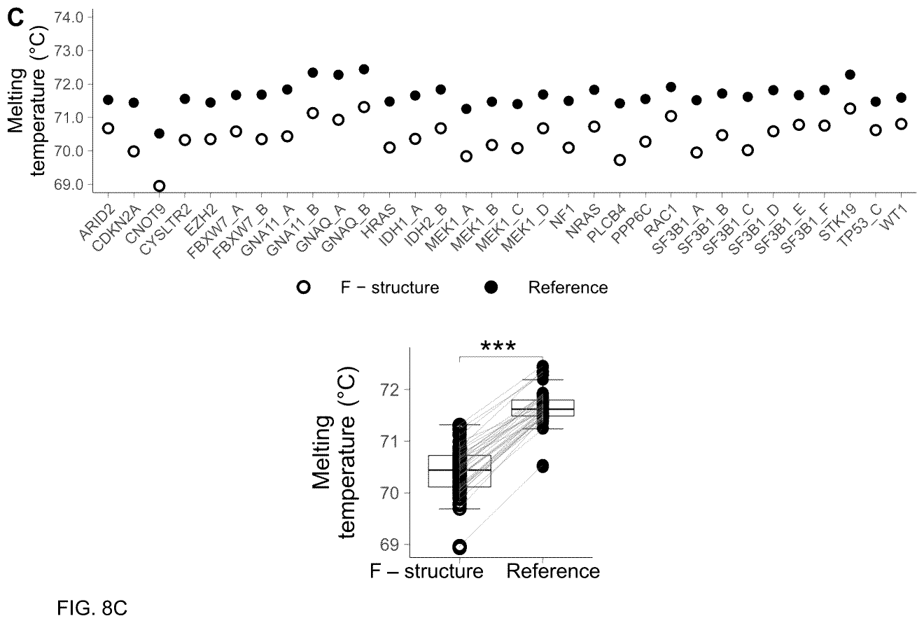

In addition to UMI design, assay performance is also dependent on the target DNA sequence of each primer. To determine if structured UMIs are superior to reference UMI, we designed 32 new assays. We used UMI design (F) and tested assay performance using quantitative PCR and capillary gel electrophoresis. FIG. 8A shows that 28 of 32 assays displayed improved assay performance with structured UMI design (F) using quantitative PCR. The mean improvement in relative specificity was 8.03. The specificity in library construction based on capillary gel electrophoresis was also improved in 28 of 32 assays with a mean improvement of from 52% to 69% in formation of specific library products (FIG. 8B). Out of these 28 assays, 25 were also superior in quantitative PCR. One assay, PPP6C, performed poorly in both qPCR and capillary gel electrophoresis.

We also determined the stem melting temperature for all 32 assays (FIG. 8C). UMI design (F) decreased the melting temperature with 1.2° C. Interestingly, we observed a correlation in melting temperature between the assays regardless UMI structure (Spearman's correlation coefficient=0.83, p<0.001). For example, if the melting temperature for UMI design (F) was high the melting temperature was also high for reference UMI for a specific assay.

### Structured UMI Design Improves the Performance of Both Smaller and Larger Multiplexes

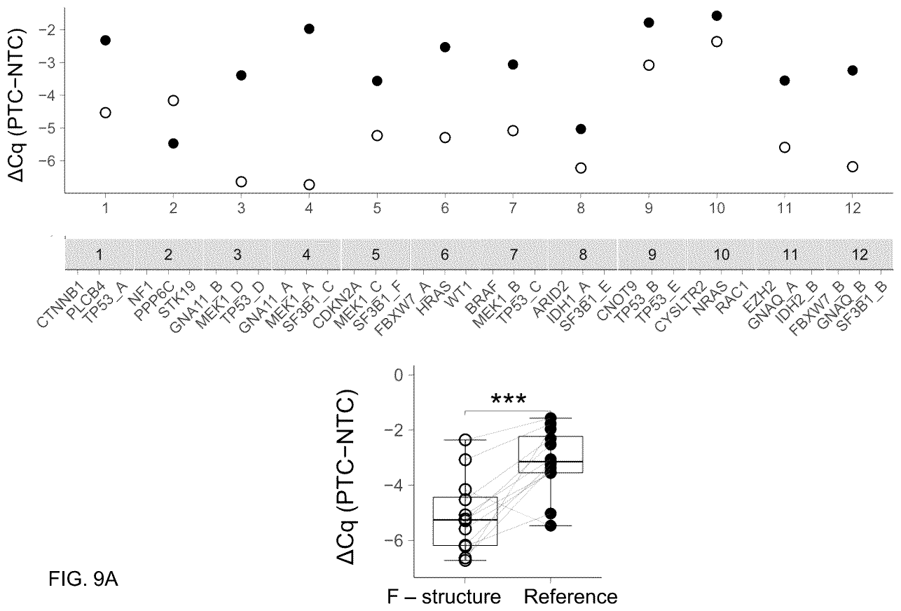

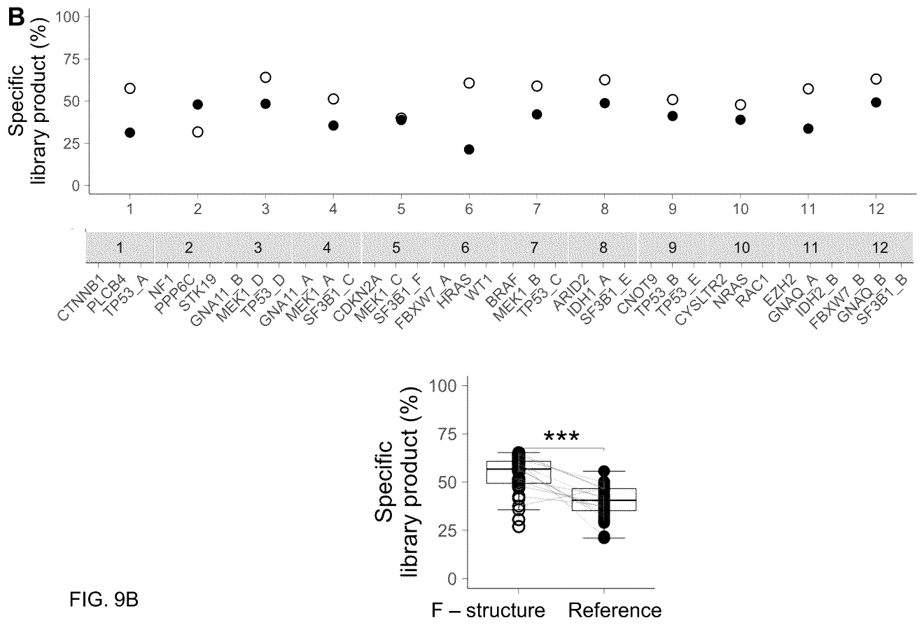

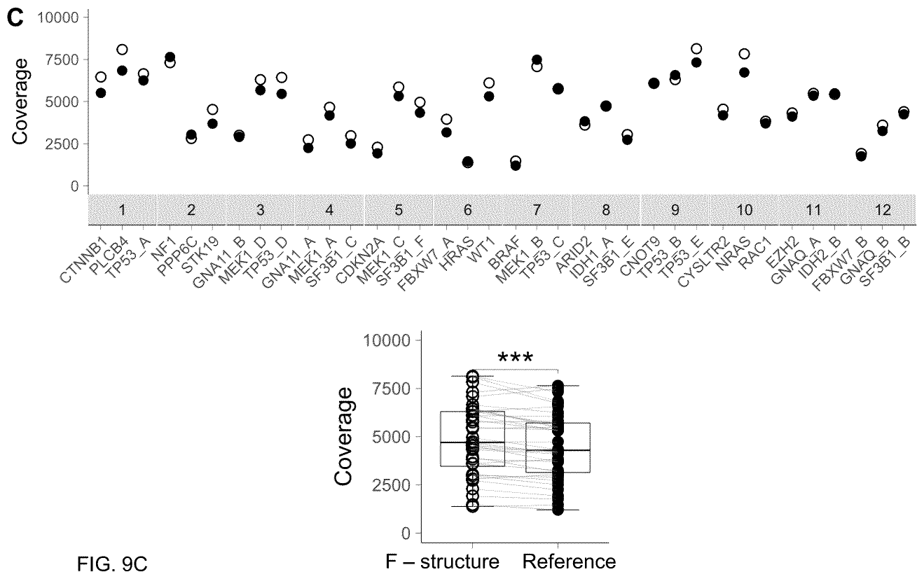

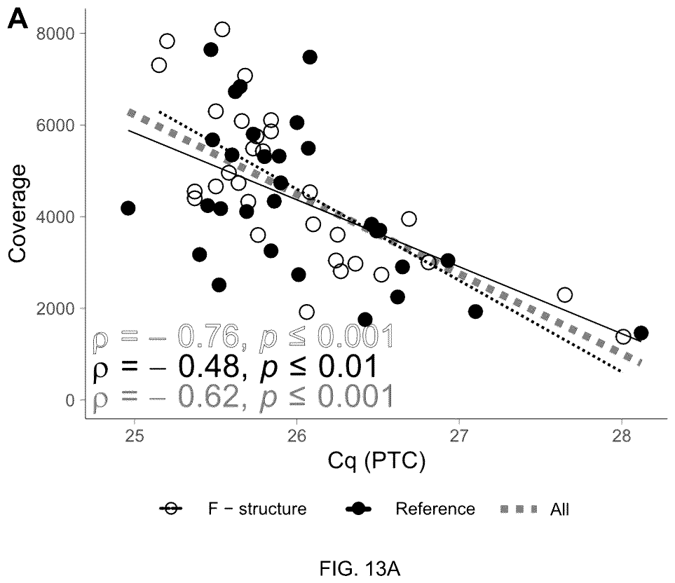

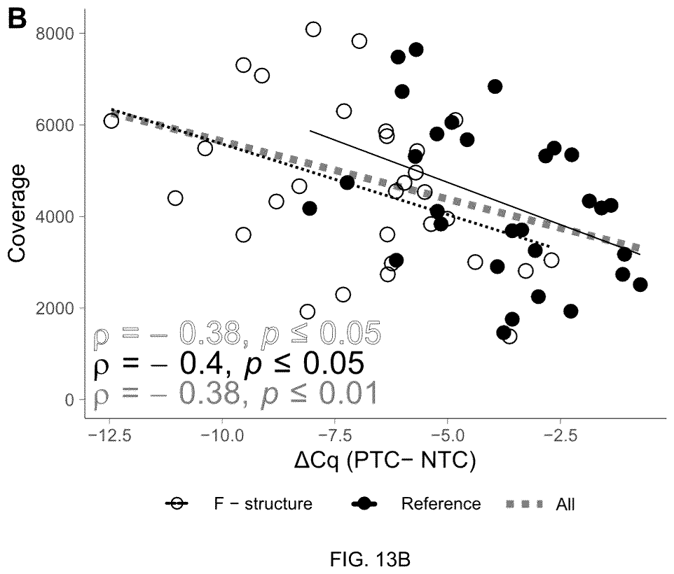

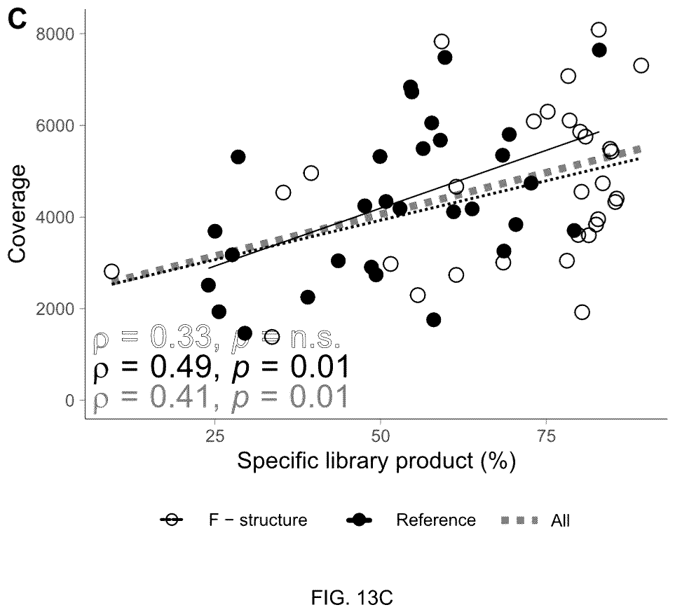

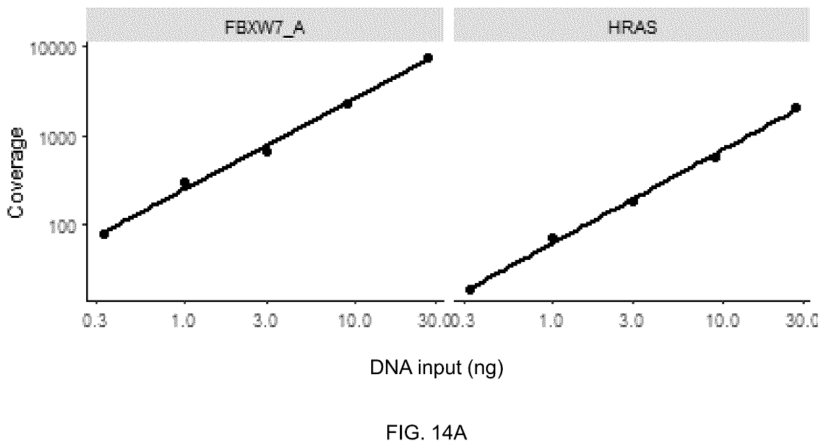

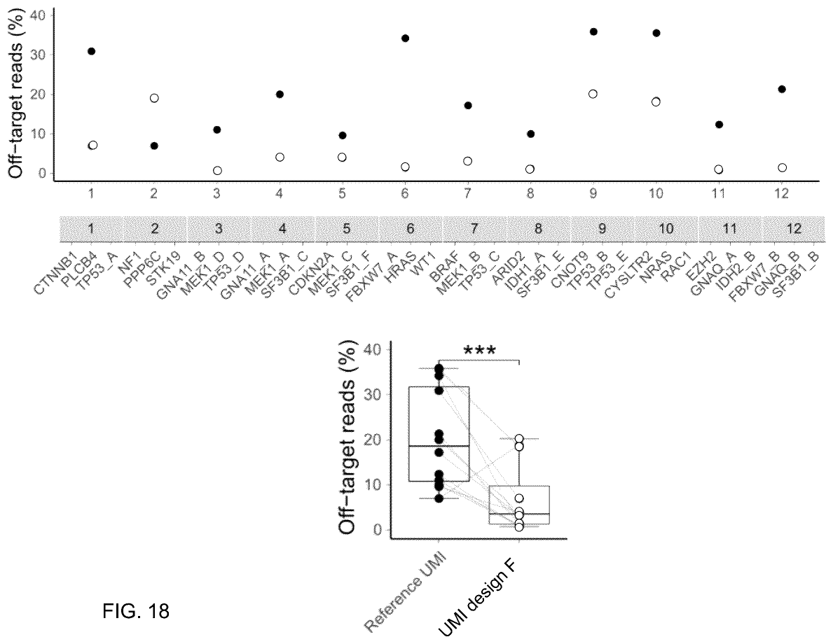

To test the performance of structured UMI design (F) in multiplexing we analyzed 12 different 3-plexes (named 1 to 12). The assays were randomly multiplexed without any overlapping amplicons. FIG. 9A shows that the relative specificities were improved in 11 of 12 3-plexes using UMI design (F) compared with reference UMI assessed by quantitative PCR. The mean improvement in relative specificity was 3.9 times. The same assays were also improved when quantifying specificity in library construction using capillary gel electrophoresis. The specific library product improved from 40% to 53%. Next, we sequenced all 3-plexes and determined each assays coverage using consensus reads compared with reference UMI. Twenty-seven out of 36 assays generated higher mean coverage using the UMI design (F). The average assay generated 7.4% higher coverage using UMI design (F) compared with reference UMI. We also compared the coverage of individual assays in 3-plexes with assay characteristics as single-plexes. Overall, all assays with high coverage in 3-plexes performed well as single-plexes as assessed by both quantitative PCR and capillary gel electrophoresis (FIG. 13). Another approach to assess assay performance is to quantify the amount of off-target sequencing reads (FIG. 18). All but one tri-plex generated lower amounts of off-target reads with UMI design F compared with reference UMI. The mean quantity of off-target reads was reduced from 20% to 6.7%.

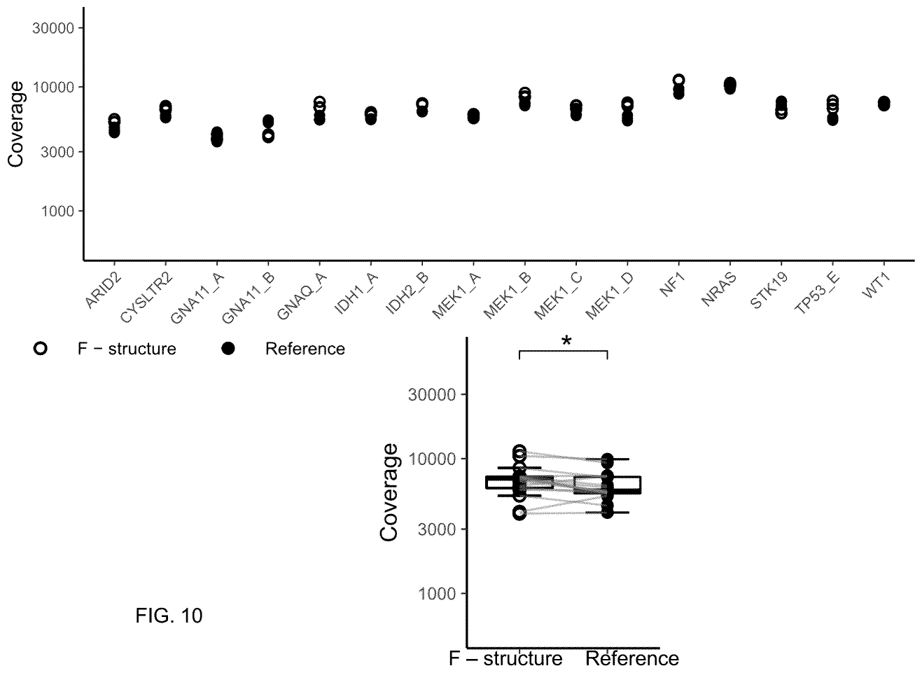

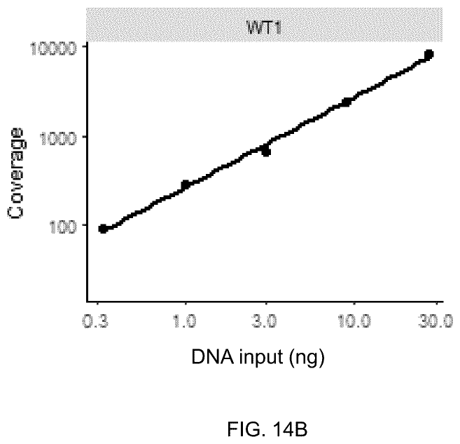

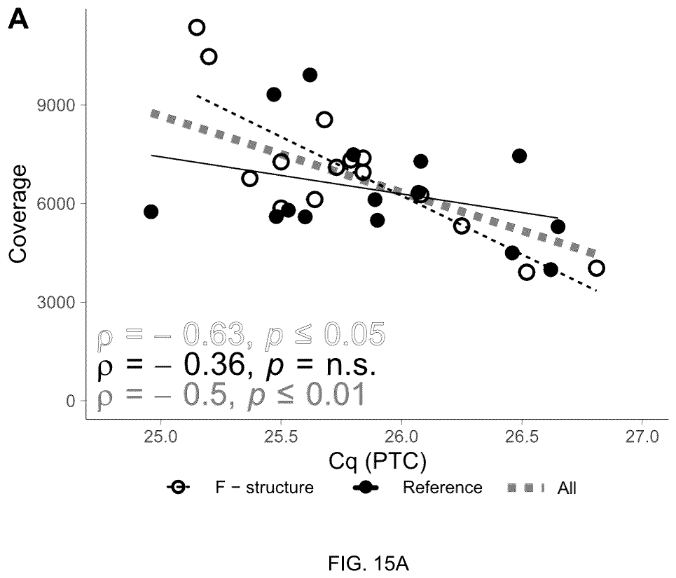

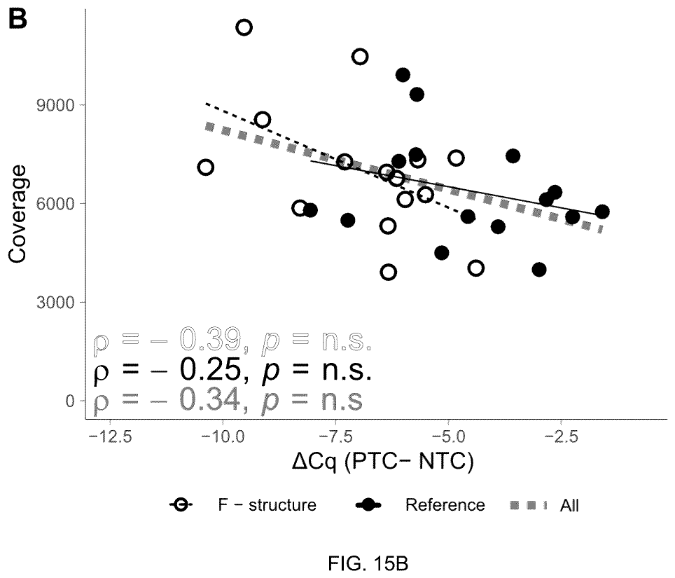

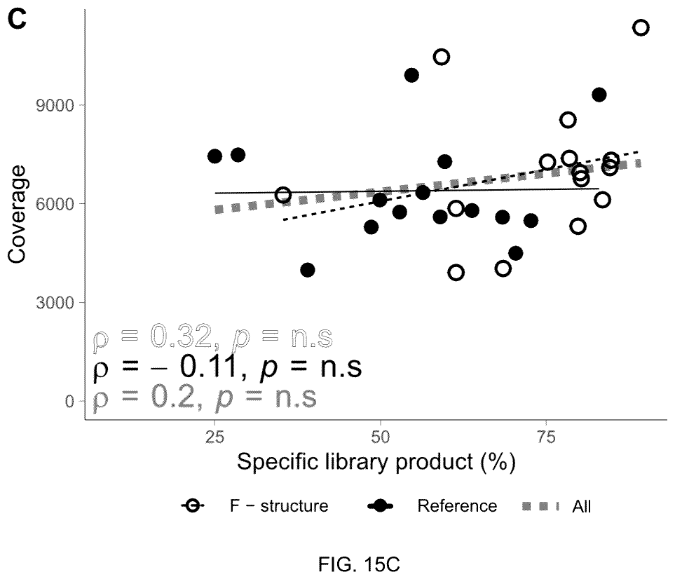

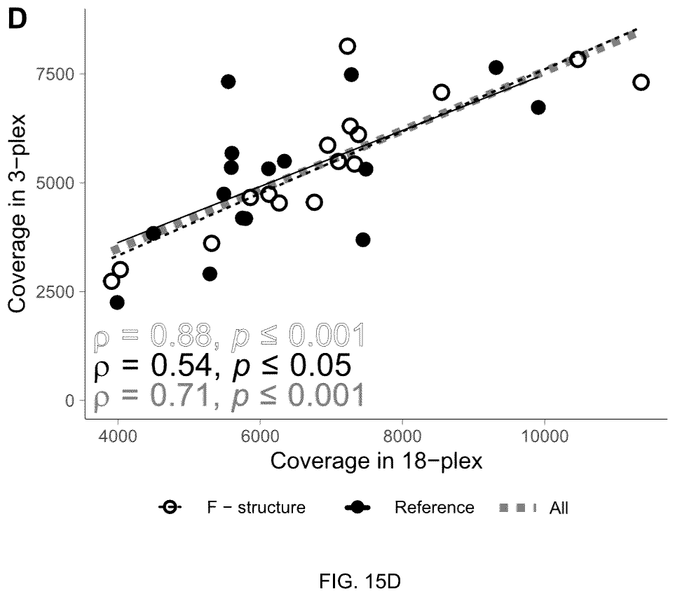

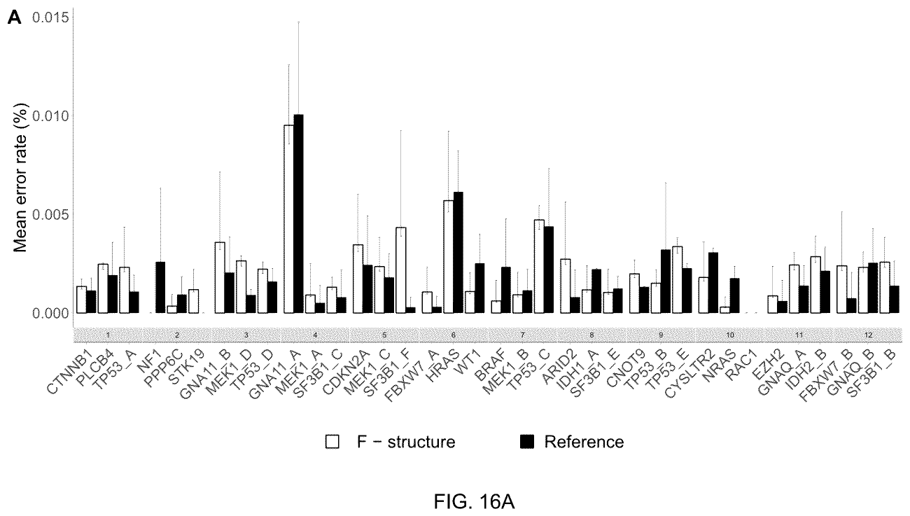

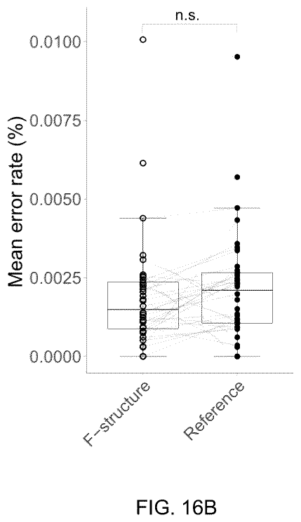

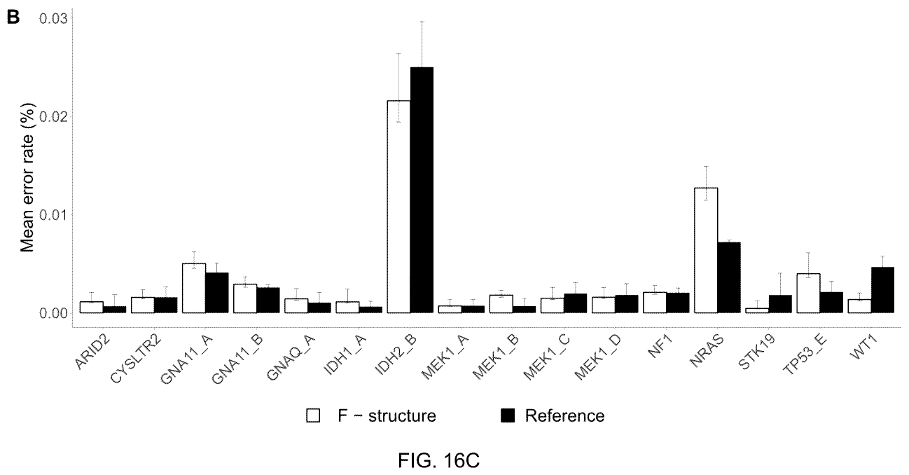

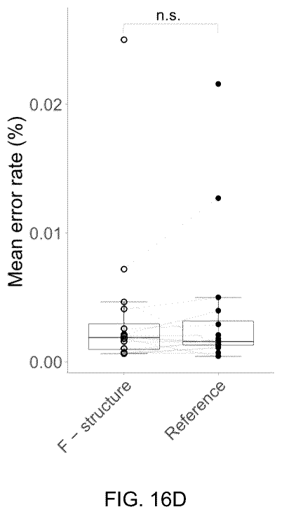

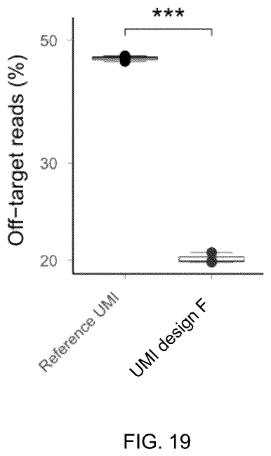

To test the ability to analyze low DNA concentration, we sequenced 3-plex 6 and UMI design (F) applying a dilution series of DNA, ranging from 27 ng to 0.33 ng (FIG. 14). All tree assays in 3-plex 6 displayed linearity between observed molecules by sequencing and loaded DNA concentrations. Finally, we sequenced a 16-plex (FIG. 10). Twelve of 16 assays generated higher coverage using UMI design (F) than reference UMI with a mean improvement of 10%. The coverage of individual assays in 16-plex correlated weaker with the assay performance of single-plexes compared with 3-plex data (FIG. 15). UMIs reduce polymerase-induced errors in sequencing. We observed no difference in errors rates comparing UMI design (F) and reference UMI, neither in the 3-plex nor 16-plex data (FIG. 16). The mean number of off-target reads was reduced from 46% to 20% using UMI design F (FIG. 19).

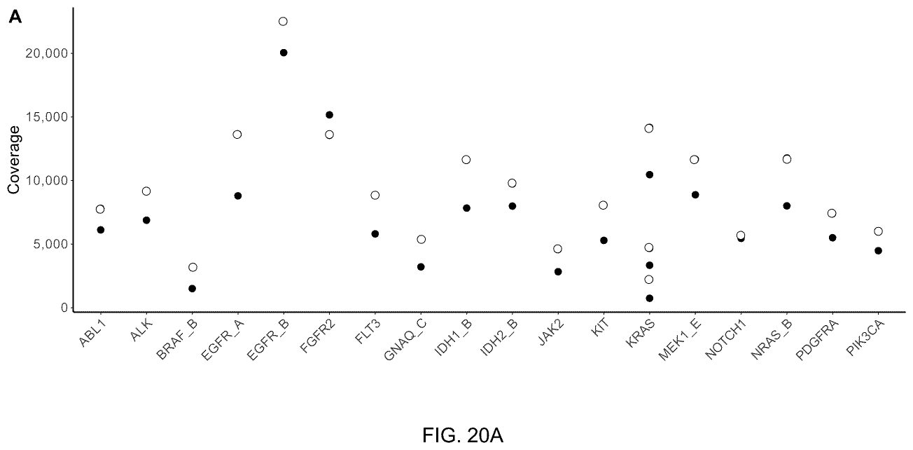

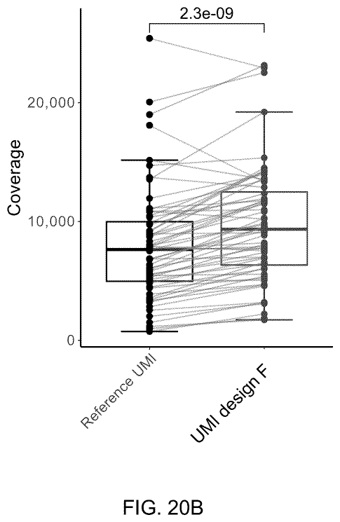

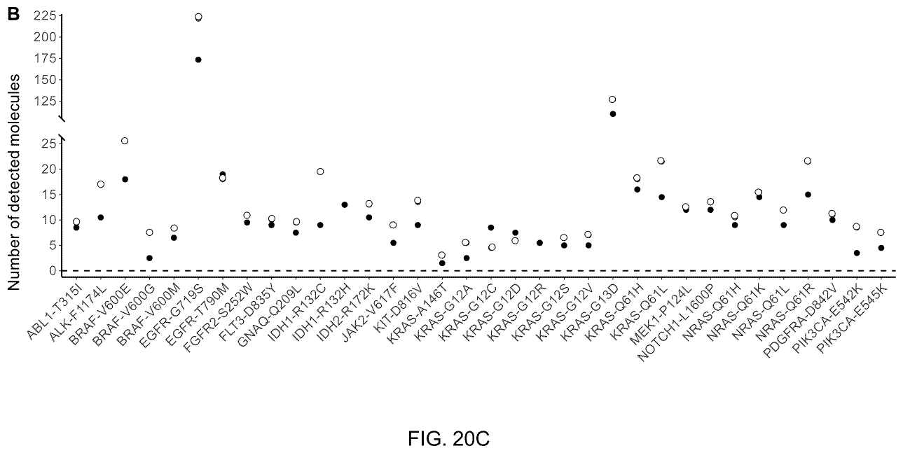

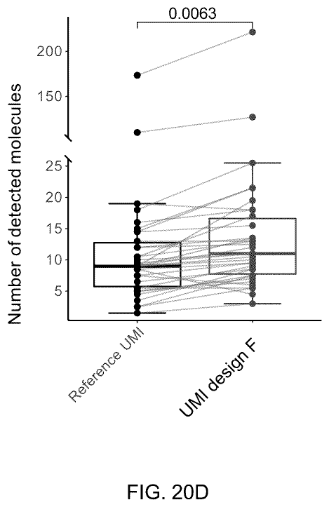

A hot-spot mutation panel consisting of 20 individual assays was analyzed using UMI design F and reference UMI. On-target amplification assessed by digital sequencing is shown in FIG. 20A. Number of consensus reads is shown for each individual assay in the hot-spot mutation panel. Mean value for each individual assay is shown, n=4. Box plots of all mean values are shown to the right. Paired Student's t-test. Panel sensitivity is shown in FIG. 20B. The number of detected mutated DNA molecules is shown, n=4. The number of mutated DNA molecules is assessed as number of consensus reads. Box plots of all mean values are shown to the right. Paired Student's t-test.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1.-23. (canceled)

24. A method of amplifying a target nucleic acid molecule in a sample, the method comprising.
contacting a sample comprising a target nucleic acid molecule with a forward primer and a reverse primer, wherein
the forward primer comprises, from a 5′ end to a 3′ end, an adapter sequence and a target-specific sequence;
the reverse primer comprises, from a 5′ end to a 3′ end, an adapter sequence and a target-specific sequence;
the forward primer is a hairpin barcode forward primer and/or the reverse primer is a hairpin barcode reverse primer;
the hairpin barcode forward primer comprises, from the 5′ end to the 3′ end, the adapter sequence, a unique molecular identifier (UMI) sequence comprising a structured UMI, and the target-specific sequence complementary to a portion of the target nucleic acid molecule;
the hairpin barcode reverse primer comprises, from the 5′ end to the 3′ end, the adapter sequence, a UMI sequence comprising a structured UMI, and the target-specific sequence complementary to a portion of the target nucleic acid molecule; and
at least a portion of the adapter sequence of the hairpin barcode forward primer and/or the hairpin barcode reverse primer is complementary to at least a portion of the UMI sequence of the hairpin barcode forward primer and/or the hairpin barcode reverse primer, the adapter sequence and the UMI sequence are configured to hybridize to each other at or under a closed annealing temperature and not hybridize to each other at or above an open annealing temperature;
amplifying the target nucleic acid molecule by performing polymerase chain reaction (PCR) pre-amplification of the nucleic acid molecule to form a plurality of barcoded PCR products, wherein the PCR pre-amplification has an annealing temperature equal to or less than the closed annealing temperature of the hairpin barcode forward primer and/or the hairpin barcode reverse primer;
contacting the plurality of barcoded PCR products with an adapter-specific forward primer and an adapter-specific reverse primer; and
amplifying the barcoded PCR products by performing PCR amplification on the barcoded PCR products to form amplified barcoded PCR products, wherein at least a portion of cycles of the PCR amplification has an annealing temperature equal to or greater than the open annealing temperature of the hairpin barcode forward primer and/or the hairpin barcode reverse primer.

25. A method for quantifying nucleic acid molecules, the method comprising:
contacting a sample comprising nucleic acid molecules with P forward primers and Q reverse primers, wherein
P is an integer equal to or larger than one and Q is an integer equal to or larger than one;
the P forward primers comprise, from a 5′ end to a 3′ end, an adapter sequence and a target-specific sequence;
the Q reverse primers comprise, from a 5′ end to a 3′ end, an adapter sequence and a target-specific sequence;
the P forward primers are P hairpin barcode forward primers and/or the Q reverse primers are Q hairpin barcode reverse primers;
each hairpin barcode forward primer comprises, from the 5′ end to the 3′ end, the adapter sequence, a unique molecular identifier (UMI) sequence comprising a structured UMI, and the target-specific sequence complementary to a respective portion of a nucleic acid molecule;
each hairpin barcode reverse primer comprises, from the 5′ end to the 3′ end, the adapter sequence, a UMI sequence comprising a structured UMI, and the target-specific sequence complementary to a respective portion of a nucleic acid molecule; and
at least a portion of the adapter sequence of the hairpin barcode forward primer and/or the hairpin barcode reverse primer is complementary to at least a portion of the UMI sequence of the hairpin barcode forward primer and/or the hairpin barcode reverse primer, the adapter sequence and the UMI sequence are configured to hybridize to each other at or under a closed annealing temperature and not hybridize to each other at or above an open annealing temperature;
amplifying the nucleic acid molecules by performing polymerase chain reaction (PCR) pre-amplification of the nucleic acid molecules to form a plurality of barcoded PCR products, wherein the PCR pre-amplification has an annealing temperature equal to or less than the closed annealing temperature of the hairpin barcode forward primers and/or the hairpin barcode reverse primers;
contacting the plurality of barcoded PCR products with an adapter-specific forward primer and an adapter-specific reverse primer;
amplifying the barcoded PCR products by performing PCR amplification on the barcoded PCR products to form a library of amplified barcoded PCR products, wherein at least a portion of cycles of the PCR amplification has an annealing temperature equal to or greater than the open annealing temperature of the hairpin barcode forward primers and/or the hairpin barcode reverse primers;
sequencing at least a respective portion of the amplified barcoded PCR products to form respective sequence reads comprising the UMI(s) and nucleic acid molecule sequence(s);
demultiplexing the sequence reads based on nucleic acid sequences of the UMIs;
mapping the demultiplexed sequence reads to respective known nucleic acid molecule sequences based on nucleic acid sequences of the nucleic acid molecule sequence(s); and
quantifying unique nucleic acid molecules based on the demultiplexed and mapped sequence reads.

26. The method according to claim 24, wherein
the hairpin barcode forward primer comprises, from the 5′ end to the 3′ end, the adapter sequence, the UMI sequence comprising the structured UMI and a stem sequence, and the target-specific sequence;
the hairpin barcode reverse primer comprises, from the 5′ end to the 3′ end, the adapter sequence, the UMI sequence comprising the structured UMI and a stem sequence, and the target-specific sequence; and
at least a portion of the adapter sequence of the hairpin barcode forward primer and/or the hairpin barcode reverse primer is complementary to at least a portion of the stem sequence of the hairpin barcode forward primer and/or the hairpin barcode reverse primer, the adapter sequence and the stem sequence are configured to hybridize to each other at or under the closed annealing temperature and not hybridize to each other at or above the open annealing temperature.

27. The method according to claim 26, wherein
the hairpin barcode forward primer comprises, from the 5′ end to the 3′ end, the adapter sequence comprising a 5′ stem sequence, the UMI sequence comprising the structured UMI and a 3′ stem sequence, and the target-specific sequence;
the hairpin barcode reverse primer comprises, from the 5′ end to the 3′ end, the adapter sequence comprising a 5′ stem sequence, the UMI sequence comprising the structured UMI and a 3′ stem sequence, and the target-specific sequence; and
at least a portion of the 5′ stem sequence of the hairpin barcode forward primer and/or the hairpin barcode reverse primer is complementary to at least a portion of the 3′ stem sequence of the hairpin barcode forward primer and/or the hairpin barcode reverse primer, the 5′ stem sequence and the 3′ stem sequence are configured to hybridize to each other at or under the closed annealing temperature and not hybridize to each other at or above the open annealing temperature.

28. The method according to claim 27, wherein the UMI sequence comprises at least one destabilizing nucleotide between the structured UMI and the 3′ stem sequence.

29. The method according to claim 24, wherein amplifying the nucleic acid molecules comprises amplifying the nucleic acid molecules by performing 1-40 of PCR pre-amplification of the nucleic acid molecules to form the plurality of barcoded PCR products.

30. The method according to claim 29, wherein amplifying the nucleic acid molecules comprises amplifying the nucleic acid molecules by performing 2-15 cycles of PCR pre-amplification of the nucleic acid molecules to form the plurality of barcoded PCR products.

31. The method according to claim 24, wherein amplifying the barcoded PCR products comprises amplifying the barcoded PCR products by performing at least 2 cycles of PCR amplification on the barcoded PCR products to form a library of amplified barcoded PCR products.

32. The method according to claim 31, wherein amplifying the barcoded PCR products comprises amplifying the barcoded PCR products by performing at least 5 cycles of PCR amplification on the barcoded PCR products to form a library of amplified barcoded PCR products.

33. The method according to claim 24, further comprising degrading a polymerase used for amplifying the nucleic acid molecules in the PCR pre-amplification prior to amplifying the barcoded PCR products in the PCR amplification.

34. The method according to claim 24, wherein the structured UMI comprises multiple UMI parts separated by a respective predefined nucleotide sequence.

35. The method according to claim 34, wherein each UMI part of the structured UMI has a same length in terms of number of nucleotides.

36. The method according to claim 34, wherein the respective predefined nucleotide sequence has a same length in terms of number of nucleotides.

37. The method according to claim 24, wherein the structured UMI comprises alternating UMI parts, each comprising one or more random nucleotides, and respective predefined nucleotide sequences, each comprising one or more predefined nucleotides.

38. The method according to claim 24, wherein the structured UMI is selected from the group consisting of:
$$\begin{matrix}{N}_{1} {M}_{1} {N}_{2} {M}_{2} {N}_{3} {M}_{3} … {N}_{k} ; \\ {N}_{1} {M}_{1} {N}_{2} {M}_{2} {N}_{3} {M}_{3} … {N}_{k} {M}_{k} ; \\ {N}_{1} … {N}_{{k}_{1}} {M}_{1} {N}_{{k}_{1} + 1} … {N}_{{k}_{2}} {M}_{2} {N}_{{k}_{2} + 1} … {N}_{{k}_{n}} ; \\ {N}_{1} … {N}_{{k}_{1}} {M}_{1} {N}_{{k}_{1} + 1} … {N}_{{k}_{2}} {M}_{2} {N}_{{k}_{2} + 1} … {N}_{{k}_{n}} {M}_{n} ; \\ {N}_{1} … {N}_{{k}_{1}} {M}_{1} … {M}_{{l}_{1}} {N}_{{k}_{1} + 1} … {N}_{{k}_{2}} {M}_{{l}_{1} + 1} … {M}_{{l}_{2}} … {N}_{{k}_{n - 1} + 1} … {N}_{{k}_{n}} ; and \\ {N}_{1} … {N}_{{k}_{1}} {M}_{1} … {M}_{{l}_{1}} {N}_{{k}_{1} + 1} … {N}_{{k}_{2}} {M}_{{l}_{1} + 1} … {M}_{{l}_{2}} … {N}_{{k}_{n - 1} + 1} … {N}_{{k}_{n}} {M}_{{l}_{n - 1} + 1} … {M}_{{l}_{n}} ,\end{matrix}$$
wherein
each N is independently a random nucleotide among A, T, C and G;
each M is independently a respective predefined nucleotide among A, T, C and G;
k is an integer from 3 up to 20;
each kᵢ, i=1 . . . n is independently an integer from 2 up to 8;
each lⱼ, j=1 . . . n is independently an integer from 1 up to 8; and
n is an integer from 3 up to 20.

39. The method according to claim 38, wherein
k is an integer from 6 up to 15;
each kᵢ, i=1 . . . n is independently an integer from 2 up to 4;
each lⱼ, j=1 . . . n is independently an integer from 1 up to 4; and
n is an integer from 6 up to 15.

40. The method according to claim 38, wherein each lⱼ is equal to or lower than each k₁.

41. The method according to claim 38, wherein the UMI is N₁ . . . Nₖ_{₁}M₁ . . . Mₗ_{₁}Nₖ_{₁}₊₁ . . . Nₖ_{₃}MNₖ_{₂}₊₁ . . . Nₖ_{₃}Mₗ_{₁}₊₁ . . . Mₗ_{₂}Nₖ_{₃}₊₁ . . . Nₖ_{₄}.

42. The method according to claim 41, wherein
each N is independently a random nucleotide among A, T, C and G; and
each M is a respective predefined nucleotide among A, T, C and G.

43. The method according to claim 41, wherein
$$\begin{matrix}{k}_{1} - 1 = {k}_{4} - {k}_{3 + 1} ; \\ {k}_{2} - {k}_{1 + 1} = {k}_{3} - {k}_{2 + 1} ; and \\ {I}_{1} - 1 = {I}_{2} - {I}_{1 + 1.}\end{matrix}$$

44. The method according to claim 41, wherein the UMI is N₁N₂N₃N₄M₁M₂M₃N₅N₆M₄N₇N₈M₅M₆M₇N₉N₁₀N₁₁N₁₂.

45. The method according to claim 24, wherein the at least a portion of the UMI sequence that is complementary to the at least a portion of the adapter sequence comprises 5-15 nucleotides.

46. The method according to claim 45, wherein the at least a portion of the UMI sequence that is complementary to the at least a portion of the adapter sequence comprises 12-15 nucleotides.

47. The method according to claim 24, wherein
the adapter-specific forward primer comprises, from a 5′ end to a 3′ end, one of a P5 sequence and a P7 sequence and a sequence equal to or complementary to at least a portion of the adapter sequence of the forward primer; and
the adapter-specific reverse primer comprises, from a 5′ end to a 3′ end, the other of the P5 sequence and the P7 sequence and a sequence equal to or complementary to at least a portion of the adapter sequence of the reverse primer.

48. The method according to claim 47, wherein
the P5 sequence comprises AATGATACGGCGACCACCGA as defined in SEQ ID NO: 6; and
the P7 sequence comprises CAAGCAGAAGACGGCATACGAGAT as defined in SEQ ID NO: 8.

49. The method according to claim 48, wherein
the P5 sequence consists of AATGATACGGCGACCACCGAGATCTACAC as defined in SEQ ID NO: 7; and
the P7 sequence consists of CAAGCAGAAGACGGCATACGAGAT as defined in SEQ ID NO: 8.

50. The method according to claim 24, wherein the adapter sequence of the forward primer is different from the adapter sequence of the reverse primer.

51. The method according to claim 24, wherein the closed annealing temperature is lower than a melting temperature of the adapter sequence and the UMI sequence.

52. The method according to claim 24, wherein the open annealing temperature is higher than a melting temperature of the adapter sequence and the UMI sequence.
